(19) United States
(12) Patent Application Publication
(10) Pub. No.: US 2022/0257775 A1
(43) Pub. Date: Aug. 18, 2022

(54) USE OF HIGHLY POTENT MULTIMERIC E-SELECTIN ANTAGONISTS FOR TREATING SICKLE CELL DISEASE

(71) Applicant: GLYCOMIMETICS, INC., Rockville, MD (US)

(72) Inventors: John L. MAGNANI, Gaithersburg, MD (US); William E. FOGLER, Lutherville-Timonium, MD (US)

(73) Assignee: GLYCOMIMETICS, INC., Rockville, MD (US)

(21) Appl. No.: 17/597,906

(22) PCT Filed: Jul. 30, 2020
(86) PCT No.: PCT/US2020/044177

§ 371 Date: Jan. 28, 2022

Related U.S. Application Data
(60) Provisional application No. 62/881,297, filed on Jul. 31, 2019.

(51) Int. Cl. A61K 47/55; A61K 47/54; A61P 7/06

(52) U.S. Cl. CPC A61K 47/55; A61P 7/06; A61K 47/549; A61K 47/545

(57) ABSTRACT

Methods for the treatment of sickle cell disease or complications associated therewith, including, for example, vaso-occlusive crisis, by the use of at least one E-selectin inhibitor and compositions comprising the same are disclosed.

This application claims the benefit under 35 U.S.C. § 119(e) to U.S. Provisional Application No. 62/881,297 filed Jul. 31, 2019, which application is incorporated by reference herein in its entirety.

Methods for the treatment of sickle cell disease or complications associated therewith by the use of at least one E-selectin inhibitor and compositions comprising the same are disclosed.

Sickle cell disease (SCD) is an inheritable hematological disorder based on a mutation in the β-globin gene of hemoglobin. It is characterized by life-long severe hemolytic anemia, recurrent pain crisis, chronic organ system damage and a marked decrease in life expectancy. Upon deoxygenation, this mutated hemoglobin polymerizes and causes a shape change (sickling) of the red blood cell. This change in red blood cells leads to obstruction of blood vessels (vaso-occlusion) causing a wide variety of complications such as stroke, pulmonary hypertension, end-organ disease and death. Vaso-occlusive phenomena and hemolysis are clinical hallmarks of SCD and can be triggered by inflammation. Vaso-occlusion results in recurrent painful episodes (sometimes called sickle cell crisis or vaso-occlusive crisis (VOC)) and a variety of serious organ system complications, among which infection, acute chest syndrome, stroke, splenic sequestration are among the most debilitating. Vaso-occlusive crisis constitutes the major morbidity in sickle cell disease. Vaso-occlusion accounts for 90% of hospitalizations in children with SCD, and it can ultimately lead to life-long disabilities and/or early death.

In addition to the fatal or potentially fatal complications, there are serious non-fatal complications of sickle cell disease such as pain. The severity of the pain may vary, but normally requires some form of medical attention. Hospitalization may be necessary.

In the U.S. alone, approximately 100,000 people suffer from sickle cell disease. Sickle cell disease is estimated to affect one of every 1,300 infants in the general population, and one of every 365 of Black or African American descent. Currently, there is no cure for sickle cell disease. The disease is chronic and lifelong. Life expectancy is typically shortened.

Accordingly, there is an unmet need in the art for compounds and compositions for treating sickle cell disease and complications associated therewith. The present invention fulfills these needs and provides related advantages as well.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

E-selectin plays a dominant role during the cellular events of vaso-occlusive crisis in sickle cell disease. Further, the importance of the function of E-selectin over P-selectin is also seen in sickle cell patients. While both E-selectin and P-selectin have been associated with vaso-occlusive crisis, the effects of E-selectin dominate, both in mouse and human models and, where tested, inhibition of E-selectin is sufficient for full inhibitory effects of vaso-occlusive crisis.

Methods for the treatment of sickle cell disease or complications associated therewith in which inhibiting binding of E-selectin to one or more E-selectin ligands may play a role are disclosed.

Disclosed are methods for the treatment of sickle cell disease or complications associated therewith, including, for example, vaso-occlusion crises, the methods comprising administering to a subject in need thereof an effective amount of at least one compound chosen from highly potent multimeric E-selectin antagonists of Formula (I):

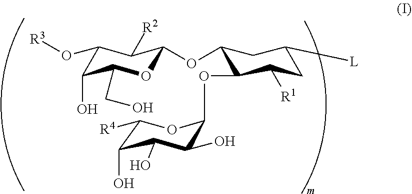

prodrugs of Formula (I), and pharmaceutically acceptable salts of any of the foregoing, wherein each R¹, R², R³, and R⁴ are defined herein.

As used herein, ‘compound of Formula (I)’ includes multimeric E-selectin antagonists of Formula (I), pharmaceutically acceptable salts of multimeric E-selectin antagonists of Formula (I), prodrugs of multimeric E-selectin antagonists of Formula (I), and pharmaceutically acceptable salts of prodrugs of multimeric E-selectin antagonists of Formula (I).

In some embodiments, a method for the treatment of sickle cell disease or complications associated therewith where inhibition of E-selectin mediated functions is useful is disclosed, the method comprising administering to a subject in need thereof an effective amount of at least one compound of Formula (I) or a pharmaceutical composition comprising at least one compound of Formula (I).

In some embodiments, a method for the treatment of vaso-occlusion crises where inhibition of E-selectin mediated functions is useful is disclosed, the method comprising administering to a subject in need thereof an effective amount of at least one compound of Formula (I) or a pharmaceutical composition comprising at least one compound of Formula (I).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the synthesis of intermediate 2.

FIG. 2 is a diagram illustrating the synthesis of intermediate 7.

FIG. 3 is a diagram illustrating the synthesis of intermediate 9.

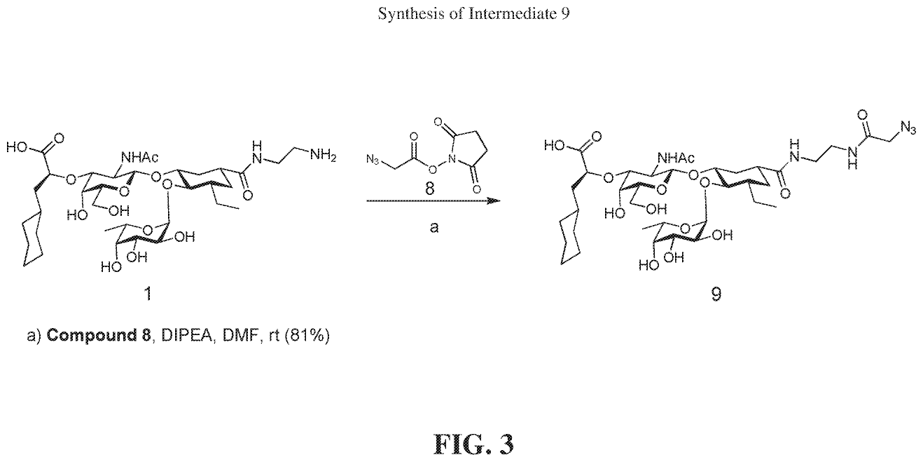

FIG. 4 is a diagram illustrating the synthesis of compound 11.

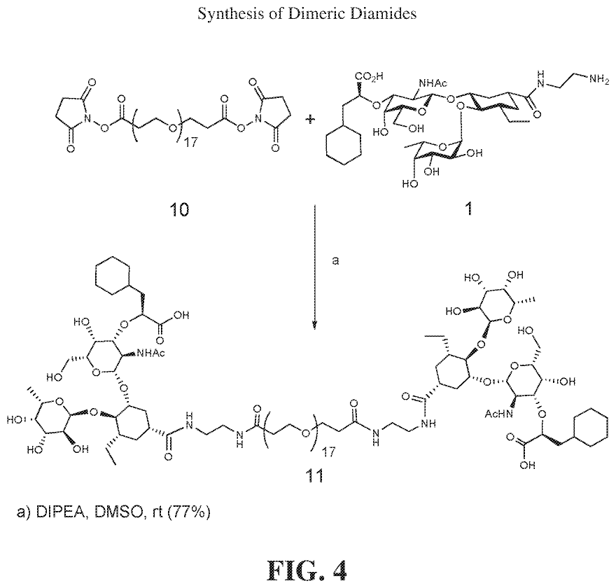

FIG. 5 is a diagram illustrating the synthesis of compound 20.

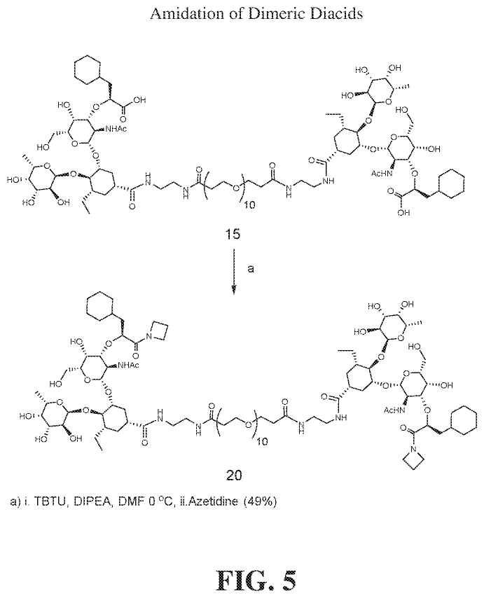

FIG. 6 is a diagram illustrating the synthesis of compound 27.

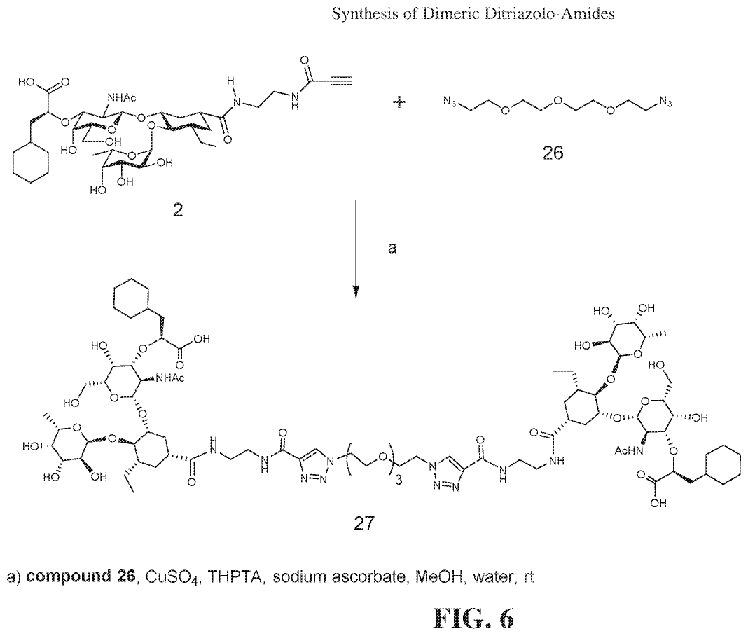

FIG. 7 is a diagram illustrating the synthesis of compound 29.

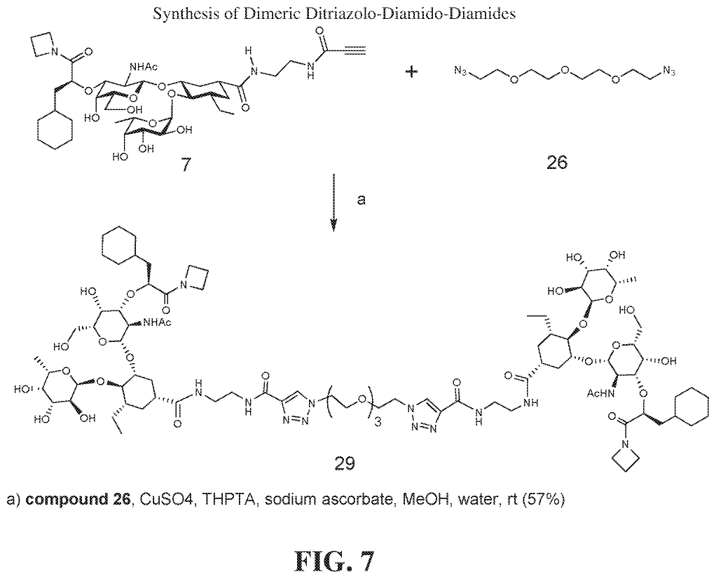

FIG. 8 is a diagram illustrating the synthesis of compound 31.

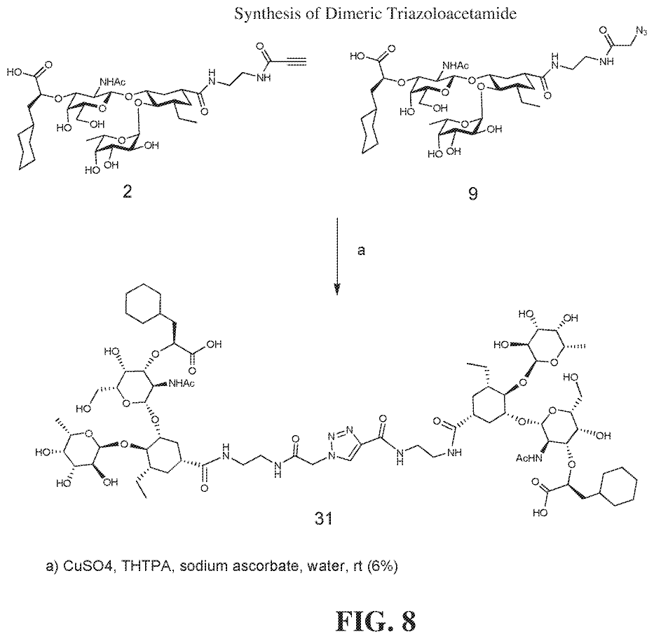

FIG. 9A is a diagram illustrating the synthesis of compound 32.

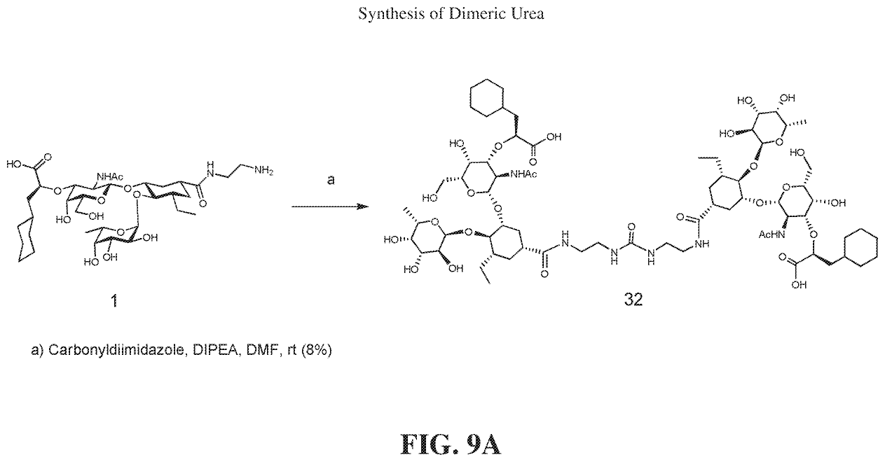

FIG. 9B is a diagram illustrating an alternative synthesis of compound 32.

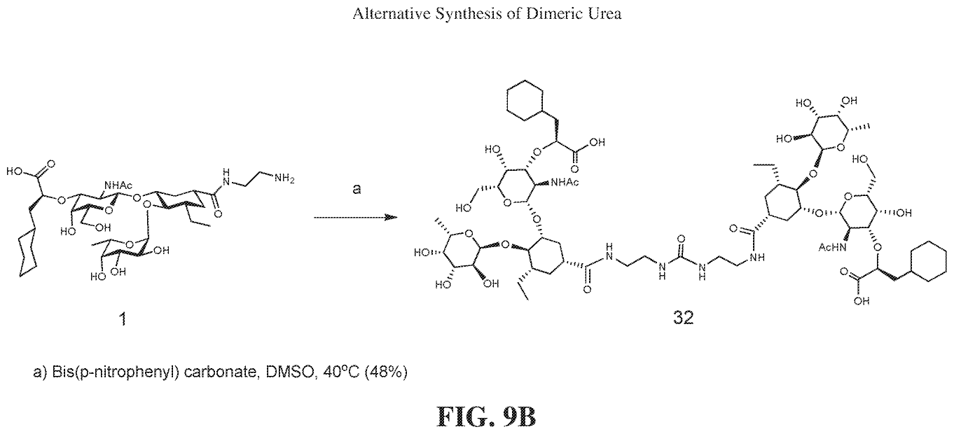

FIG. 10 is a diagram illustrating the synthesis of compound 35.

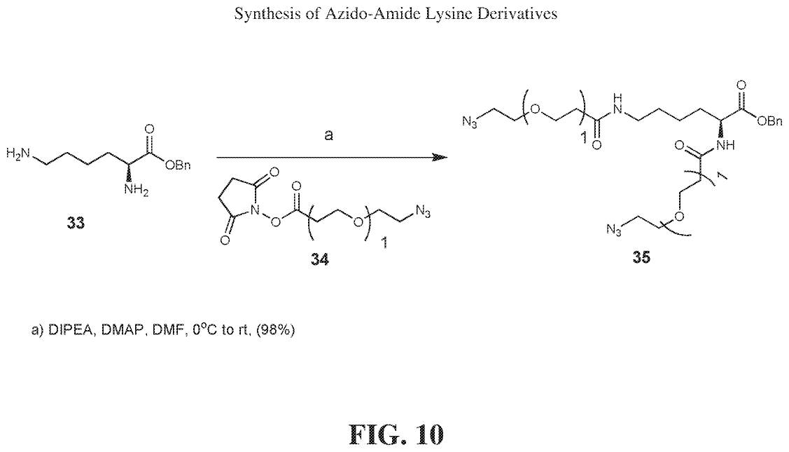

FIG. 11 is a diagram illustrating the synthesis of compound 38.

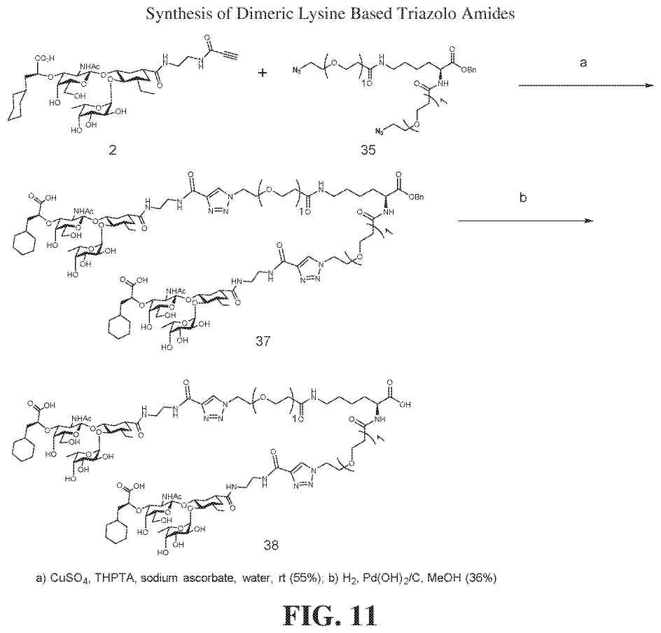

FIG. 12 is a diagram illustrating the synthesis of compound 42.

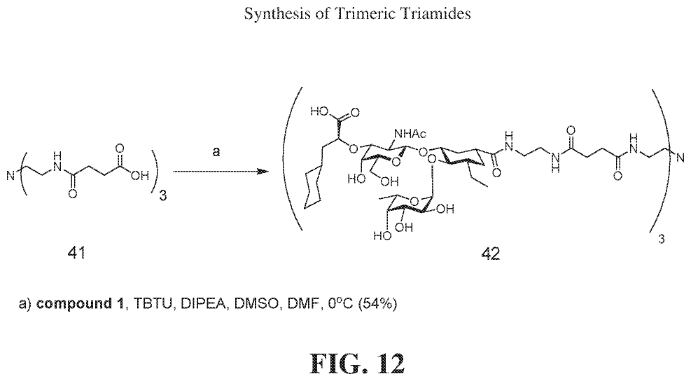

FIG. 13 is a diagram illustrating the synthesis of compound 44.

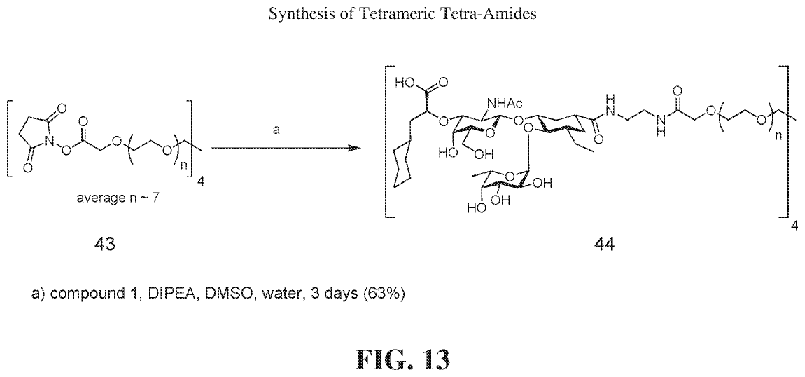

FIG. 14 is a diagram illustrating the synthesis of compound 45.

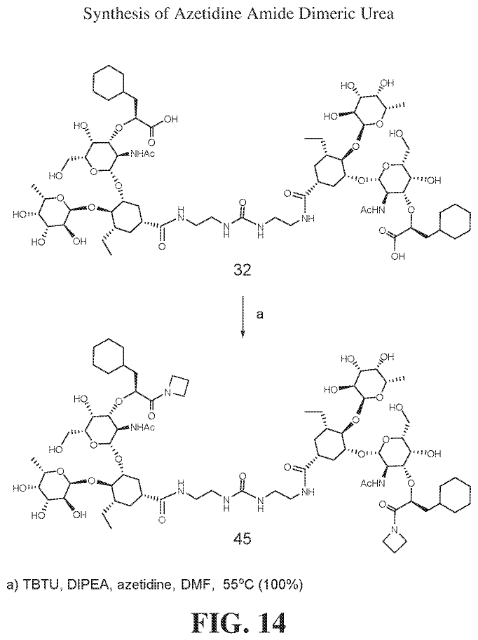

FIG. 15 is a table illustrating the bioavailability of compound 45.

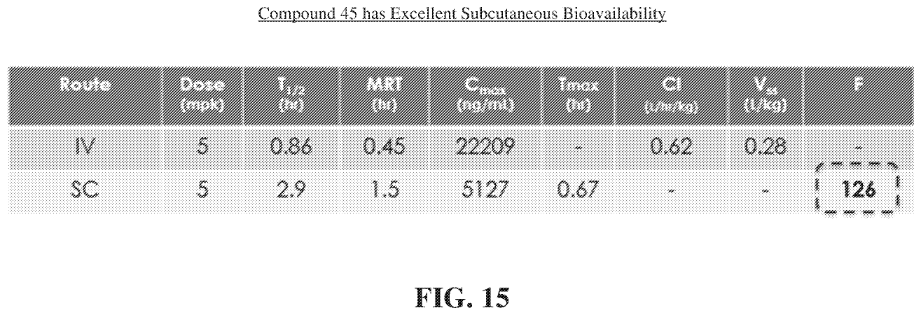

FIG. 16 is a graph illustrating the effect that E-selectin and P-selectin have on inducing transition from rolling to arrest.

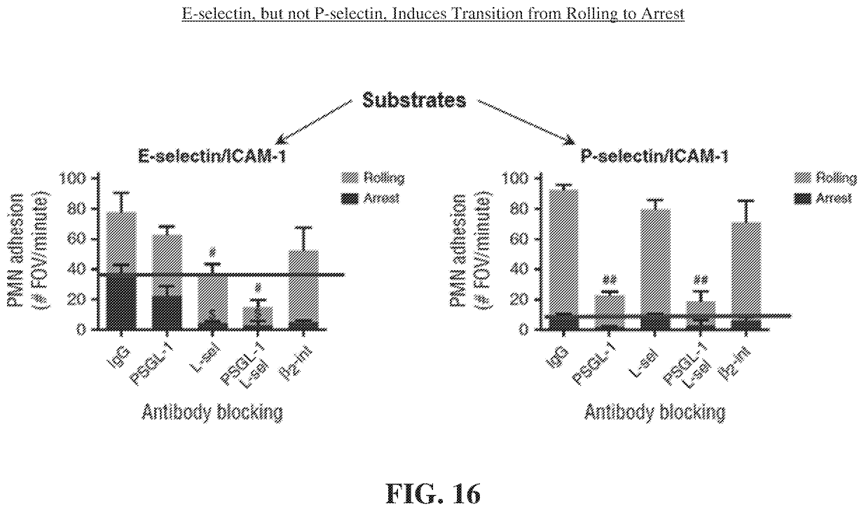

FIG. 17 is a diagram illustrating compound 45 SCD intravital microscopy and adhesion experimental procedure in nude mice.

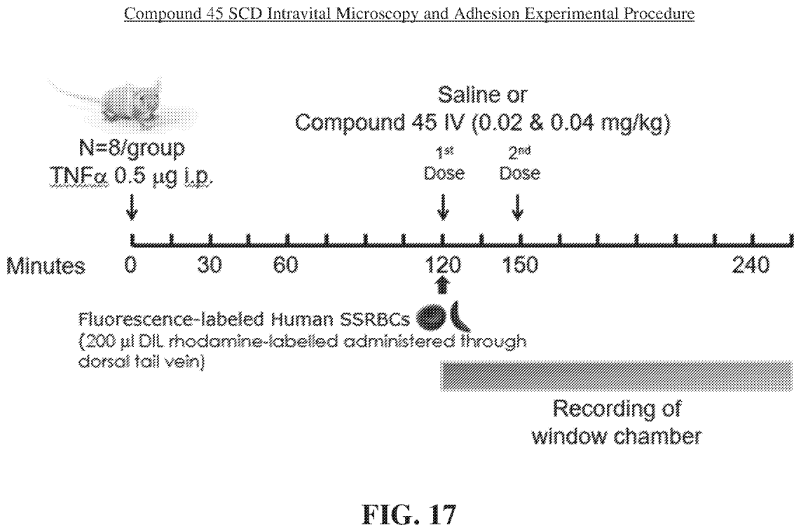

FIG. 18 is a graph illustrating that compound 45 improves blood flow in inflamed vessels in nude mice.

FIG. 19 is a graph illustrating that compound 45 reduces human SSRBC adhesion in nude mice after the inflammatory trigger of vaso-occlusion.

FIG. 20 is a graph illustrating the effect of compound 45 on the number of circulating human SSRBCs.

FIG. 21 is a graph illustrating compound 45 SCD intravital microscopy and adhesion experimental procedure in Townes mice.

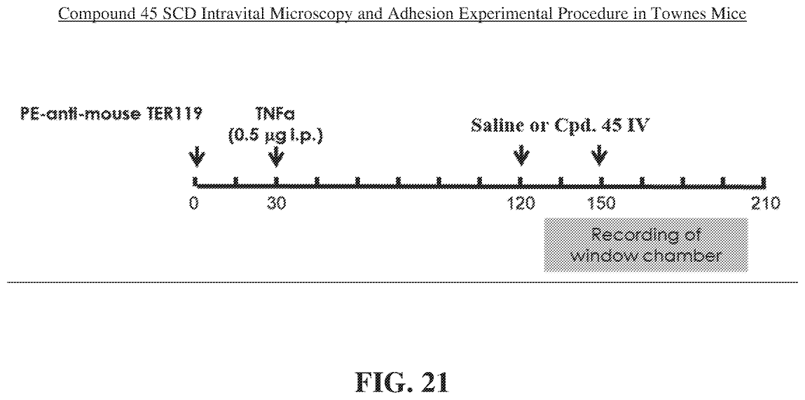

FIG. 22 is a graph illustrating that compound 45 reduces SSRBC adhesion in Townes mice after the inflammatory trigger of vaso-occlusion.

FIG. 23 is a graph illustrating that compound 45 improves blood flow in inflamed vessels in Townes mice.

Disclosed herein are methods for the treatment of sickle cell disease or complications associated therewith, including, for example, vaso-occlusive crisis, the methods comprising administering to a subject in need thereof an effective amount of E-selectin antagonists or pharmaceutical compositions comprising the same.

The compounds used in the methods of the present disclosure have been found to be highly potent multimeric E-selectin antagonists, the potency being many times greater than the monomer.

In some embodiments, presented are methods for the treatment of sickle cell disease or complications associated therewith, the methods comprising administering to a subject in need thereof an effective amount of at least one compound chosen from highly potent multimeric E-selectin antagonists of Formula (I):

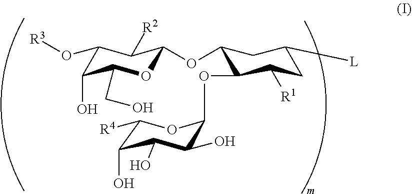

prodrugs of Formula (I), and pharmaceutically acceptable salts of any of the foregoing, wherein

each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;

each R², which may be identical or different, is independently chosen from halo, —OY¹,

—NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;

each R³, which may be identical or different, is independently chosen from

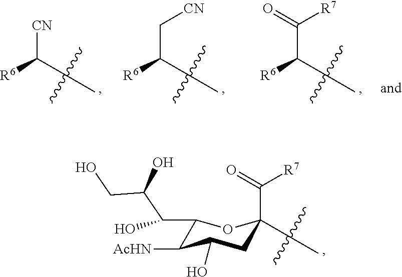

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;

each R⁴, which may be identical or different, is independently chosen from —CN, C₁₋₄ alkyl, and C₁₋₄ haloalkyl groups;

m is chosen from integers ranging from 2 to 256; and

L is chosen from linker groups.

In some embodiments, the at least one compound is chosen from compounds of Formula (I):

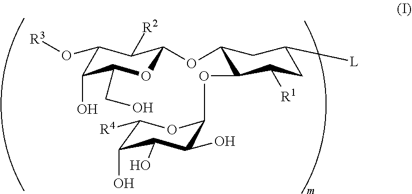

prodrugs of Formula (I), and pharmaceutically acceptable salts of any of the foregoing, wherein

each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;

each R², which may be identical or different, is independently chosen from halo, —OY¹,

—NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;

each R³, which may be identical or different, is independently chosen from

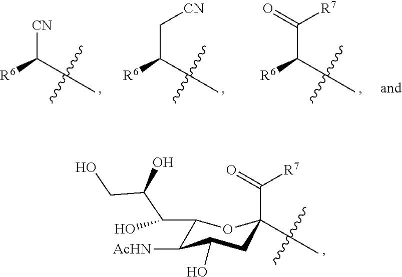

wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;

each R⁴, which may be identical or different, is independently chosen from —CN, C₁₋₄ alkyl, and C₁₋₄ haloalkyl groups;

m is chosen from integers ranging from 2 to 256; and

L is chosen from linker groups;

with the proviso that when m is 4, each R¹ and each R⁴ is methyl, each R² is —OC(═O)Ph, and each R³ is

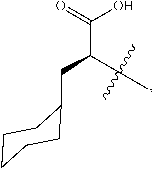 [SMILES: *[C@@H](CC1CCCCC1)C(=O)O]

then the linker groups are not chosen from

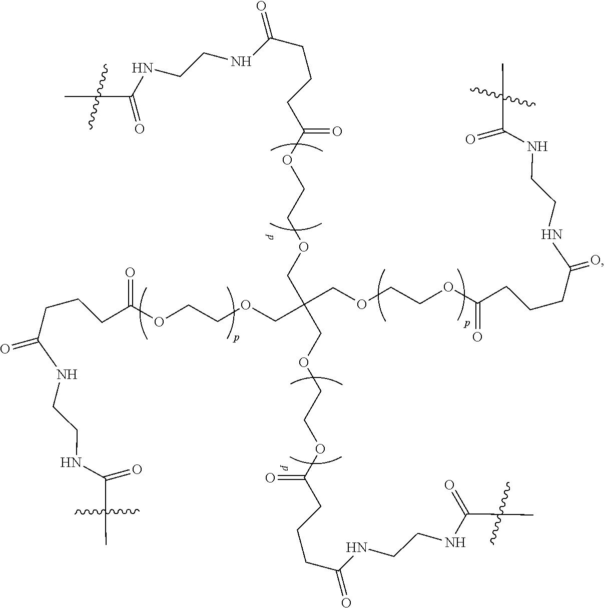

wherein p is chosen from integers ranging from 0 to 250.

In some embodiments, at least one R¹ is H. In some embodiments, at least one R¹ is chosen from C₁₋₁₂ alkyl groups. In some embodiments, at least one R¹ is chosen from C₁₋₆ alkyl groups. In some embodiments, at least one R¹ is methyl. In some embodiments, at least one R¹ is ethyl.

In some embodiments, each R¹ is H. In some embodiments, each R¹, which may be identical or different, is independently chosen from C₁₋₁₂ alkyl groups. In some embodiments, each R¹, which may be identical or different, is independently chosen from C₁₋₆ alkyl groups. In some embodiments, each R¹ is identical and chosen from C₁₋₆ alkyl groups. In some embodiments, each R¹ is methyl. In some embodiments, each R¹ is ethyl.

In some embodiments, at least one R¹ is chosen from —NHC(═O)R⁵ groups. In some embodiments, each R¹ is chosen from —NHC(═O)R⁵ groups. In some embodiments, at least one R⁵ is chosen from H, C₁₋₈ alkyl, C₆₋₁₈ aryl, and C₁₋₁₃ heteroaryl groups. In some embodiments, each R⁵ is chosen from H, C₁₋₈ alkyl, C₆₋₁₈ aryl, and C₁₋₁₃ heteroaryl groups. In some embodiments, at least one R⁵ is chosen from

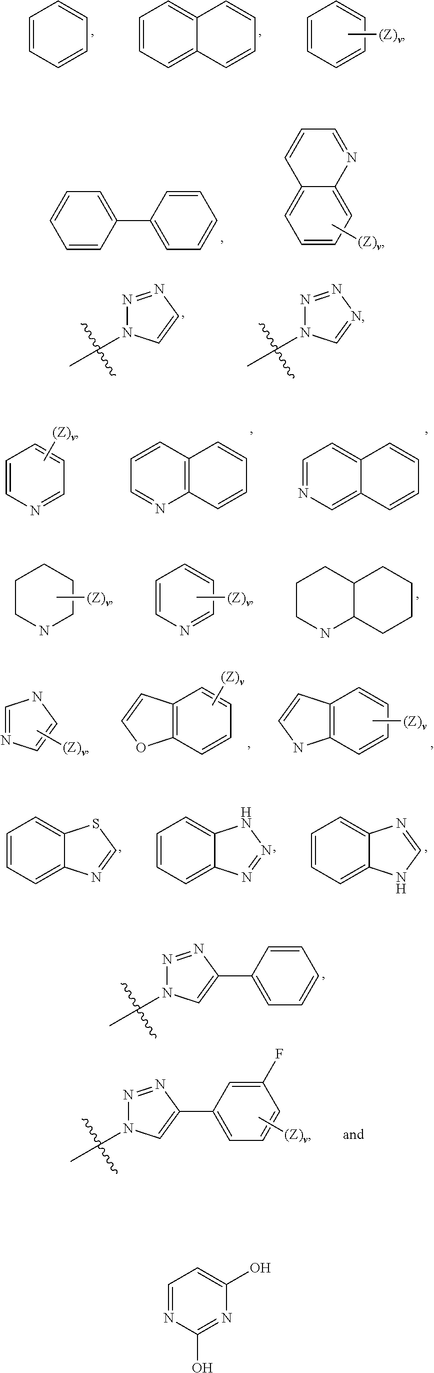

groups, wherein each Z is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein v is chosen from integers ranging from 0 to 3.

In some embodiments, at least one R² is chosen from halo groups. In some embodiments, at least one R² is fluoro. In some embodiments, at least one R² is chloro. In some embodiments, at least one R² is chosen from —OY¹ groups. In some embodiments, at least one R² is —OH. In some embodiments, at least one R² is chosen from —NY¹Y² groups. In some embodiments, at least one R² is chosen from —OC(═O)Y¹ groups. In some embodiments, at least one R² is chosen from —NHC(═O)Y¹ groups. In some embodiments, at least one R² is chosen from —NHC(═O)NY¹Y² groups.

In some embodiments, each R², which may be identical or different, is independently chosen from halo groups. In some embodiments, each R² is fluoro. In some embodiments, each R² is chloro. In some embodiments, each R², which may be identical or different, is independently chosen from —OY¹ groups. In some embodiments, each R² is —OH. In some embodiments, each R², which may be identical or different, is independently chosen from

—NY¹Y² groups. In some embodiments, each R², which may be identical or different, is independently chosen from —OC(═O)Y¹ groups. In some embodiments, each R², which may be identical or different, is independently chosen from —NHC(═O)Y¹ groups. In some embodiments, each R², which may be identical or different, is independently chosen from —NHC(═O)NY¹Y² groups. In some embodiments, each R² is identical and chosen from —OY¹ groups. In some embodiments, each R² is identical and chosen from —NY¹Y² groups. In some embodiments, each R² is identical and chosen from —OC(═O)Y¹ groups. In some embodiments, each R² is identical and chosen from —NHC(═O)Y¹ groups. In some embodiments, each R² is identical and chosen from —NHC(═O)NY¹Y² groups.

In some embodiments, at least one Y¹ and/or at least one Y² is chosen from H, C₁₋₁₂ alkyl, C₆₋₁₈ aryl, and C₁₋₁₃ heteroaryl groups. In some embodiments, at least one Y¹ and/or at least one Y² is H. In some embodiments, at least one Y¹ and/or at least one Y² is chosen from C₁₋₁₂ alkyl groups. In some embodiments, at least one Y¹ and/or at least one Y² is chosen from C₁₋₈ alkyl groups. In some embodiments, at least one Y¹ and/or at least one Y² is chosen from C₁₋₄ alkyl groups. In some embodiments, at least one Y¹ and/or at least one Y² is chosen from C₆₋₁₈ aryl groups. In some embodiments, at least one Y¹ and/or at least one Y² is chosen from C₆₋₁₂ aryl groups. In some embodiments, at least one Y¹ and/or at least one Y² is chosen from C₆₋₁₀ aryl groups. In some embodiments, at least one Y¹ and/or at least one Y² is chosen from C₁₋₁₃ heteroaryl groups. In some embodiments, at least one Y¹ and/or at least one Y² is chosen from C₁₋₉ heteroaryl groups. In some embodiments, at least one Y¹ and/or at least one Y² is chosen from C₁₋₅ heteroaryl groups. In some embodiments, at least one Y¹ and/or at least one Y² is chosen from C₁₋₃ heteroaryl groups.

In some embodiments, each Y¹, which may be identical or different, is independently chosen from H, C₁₋₁₂ alkyl, C₆₋₁₈ aryl, and C₁₋₁₃ heteroaryl groups. In some embodiments, each Y¹ is H. In some embodiments, each Y¹, which may be identical or different, is independently chosen from C₁₋₁₂ alkyl groups. In some embodiments, each Y¹, which may be identical or different, is independently chosen from C₁₋₈ alkyl groups. In some embodiments, each Y¹, which may be identical or different, is independently chosen from C₁₋₄ alkyl groups. In some embodiments, each Y¹, which may be identical or different, is independently chosen from C₆₋₁₈ aryl groups. In some embodiments, each Y¹, which may be identical or different, is independently chosen from C₆₋₁₂ aryl groups. In some embodiments, each Y¹, which may be identical or different, is independently chosen from C₆₋₁₀ aryl groups. In some embodiments, each Y¹, which may be identical or different, is independently chosen from C₁₋₁₃ heteroaryl groups. In some embodiments, each Y¹, which may be identical or different, is independently chosen from C₁₋₉ heteroaryl groups. In some embodiments, each Y¹, which may be identical or different, is independently chosen from C₁₋₅ heteroaryl groups. In some embodiments, each Y¹, which may be identical or different, is independently chosen from C₁₋₃ heteroaryl groups.

In some embodiments, each Y², which may be identical or different, is independently chosen from H, C₁₋₁₂ alkyl, C₆₋₁₈ aryl, and C₁₋₁₃ heteroaryl groups. In some embodiments, each Y² is H. In some embodiments, each Y², which may be identical or different, is independently chosen from C₁₋₁₂ alkyl groups. In some embodiments, each Y², which may be identical or different, is independently chosen from C₁₋₈ alkyl groups. In some embodiments, each Y², which may be identical or different, is independently chosen from C₁₋₄ alkyl groups. In some embodiments, each Y², which may be identical or different, is independently chosen from C₆₋₁₈ aryl groups. In some embodiments, each Y², which may be identical or different, is independently chosen from C₆₋₁₂ aryl groups. In some embodiments, each Y², which may be identical or different, is independently chosen from C₆₋₁₀ aryl groups. In some embodiments, each Y², which may be identical or different, is independently chosen from C₁₋₁₃ heteroaryl groups. In some embodiments, each Y², which may be identical or different, is independently chosen from C₁₋₉ heteroaryl groups. In some embodiments, each Y², which may be identical or different, is independently chosen from C₁₋₅ heteroaryl groups. In some embodiments, each Y², which may be identical or different, is independently chosen from C₁₋₃ heteroaryl groups.

In some embodiments, each Y¹ is identical and chosen from H, C₁₋₁₂ alkyl, C₆₋₁₈ aryl, and C₁₋₁₃ heteroaryl groups. In some embodiments, each Y¹ is identical and chosen from C₁₋₁₂ alkyl groups. In some embodiments, each Y¹ is identical and chosen from C₁₋₈ alkyl groups. In some embodiments, each Y¹ is identical and chosen from C₁₋₄ alkyl groups. In some embodiments, each Y¹ is identical and chosen from C₆₋₁₈ aryl groups. In some embodiments, each Y¹ is identical and chosen from C₆₋₁₂ aryl groups. In some embodiments, each Y¹ is identical and chosen from C₆₋₁₀ aryl groups. In some embodiments, each Y¹ is identical and chosen from C₁₋₁₃ heteroaryl groups. In some embodiments, each Y¹ is identical and chosen from C₁₋₉ heteroaryl groups. In some embodiments, each Y¹ is identical and chosen from C₁₋₅ heteroaryl groups. In some embodiments, each Y¹ is identical and chosen from C₁₋₃ heteroaryl groups.

In some embodiments, each Y² is identical and chosen from H, C₁₋₁₂ alkyl, C₆₋₁₈ aryl, and C₁₋₁₃ heteroaryl groups. In some embodiments, each Y² is identical and chosen from C₁₋₁₂ alkyl groups. In some embodiments, each Y² is identical and chosen from C₁₋₈ alkyl groups. In some embodiments, each Y² is identical and chosen from C₁₋₄ alkyl groups. In some embodiments, each Y² is identical and chosen from C₆₋₁₈ aryl groups. In some embodiments, each Y² is identical and chosen from C₆₋₁₂ aryl groups. In some embodiments, each Y² is identical and chosen from C₆₋₁₀ aryl groups. In some embodiments, each Y² is identical and chosen from C₁₋₁₃ heteroaryl groups. In some embodiments, each Y² is identical and chosen from C₁₋₉ heteroaryl groups. In some embodiments, each Y² is identical and chosen from C₁₋₅ heteroaryl groups. In some embodiments, each Y² is identical and chosen from C₁₋₃ heteroaryl groups.

In some embodiments, at least one Y¹ is methyl. In some embodiments, at least one Y¹ is phenyl. In some embodiments, each Y¹ is methyl. In some embodiments, each Y¹ is phenyl. In some embodiments, at least one Y¹ is methyl and at least one Y² is H. In some embodiments, at least one Y¹ is phenyl and at least one Y² is H. In some embodiments, each Y¹ is methyl and each Y² is H. In some embodiments, each Y¹ is phenyl and each Y² is H.

In some embodiments, at least one R² is chosen from

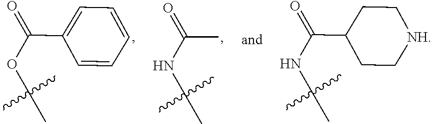

In some embodiments, each R² is

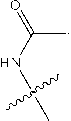

In some embodiments, each R² is

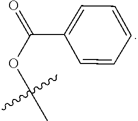 [SMILES: *OC(=O)c1ccccc1]

In some embodiments, each R² is

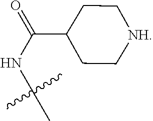 [SMILES: *NC(=O)C1CCNCC1]

In some embodiments, at least one R³, which may be identical or different, is independently chosen from

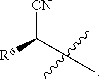

In some embodiments, at least one R³, which may be identical or different, is independently chosen from

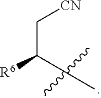

In some embodiments, at least one R³, which may be identical or different, is independently chosen from

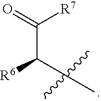

In some embodiments, at least one R³ is

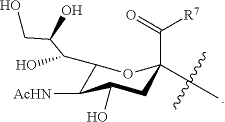

In some embodiments, each R³, which may be identical or different, is independently chosen from

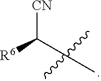

In some embodiments, each R³, which may be identical or different, is independently chosen from

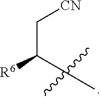

In some embodiments, each R³, which may be identical or different, is independently chosen from

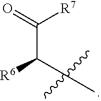

In some embodiments, each R³ is

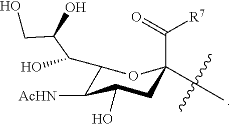

In some embodiments, each R³ is identical and chosen from

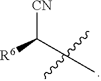

In some embodiments, each R³ is identical and chosen from

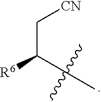

In some embodiments, each R³ is identical and chosen from

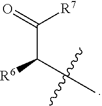

In some embodiments, each R⁶, which may be identical or different, is independently chosen from C₁₋₁₂ alkyl and C₁₋₁₂ haloalkyl groups. In some embodiments, each R⁶, which may be identical or different, is independently chosen from C₁₋₁₂ alkyl groups. In some embodiments, each R⁶, which may be identical or different, is independently chosen from C₁₋₈ alkyl groups. In some embodiments, each R⁶, which may be identical or different, is independently chosen from C₁₋₅ alkyl groups. In some embodiments, each R⁶, which may be identical or different, is independently chosen from C₂₋₄ alkyl groups. In some embodiments, each R⁶, which may be identical or different, is independently chosen from C₂₋₇ alkyl groups. In some embodiments, each R⁶, which may be identical or different, is independently chosen from C₁₋₁₂ haloalkyl groups. In some embodiments, each R⁶, which may be identical or different, is independently chosen from C₁₋₈ haloalkyl groups. In some embodiments, each R⁶, which may be identical or different, is independently chosen from C₁₋₅ haloalkyl groups.

In some embodiments, each R⁶ is identical and chosen from C₁₋₁₂ alkyl and C₁₋₁₂ haloalkyl groups. In some embodiments, each R⁶ is identical and chosen from C₁₋₁₂ alkyl groups. In some embodiments, each R⁶ is identical and chosen from C₁₋₈ alkyl groups. In some embodiments, each R⁶ is identical and chosen from C₁₋₅ alkyl groups. In some embodiments, each R⁶ is identical and chosen from C₂₋₄ alkyl groups. In some embodiments, each R⁶ is identical and chosen from C₂₋₇ alkyl groups. In some embodiments, each R⁶ is identical and chosen from C₁₋₁₂ haloalkyl groups. In some embodiments, each R⁶ is identical and chosen from C₁₋₈ haloalkyl groups. In some embodiments, each R⁶ is identical and chosen from C₁₋₅ haloalkyl groups.

In some embodiments, at least one R⁶ is chosen from

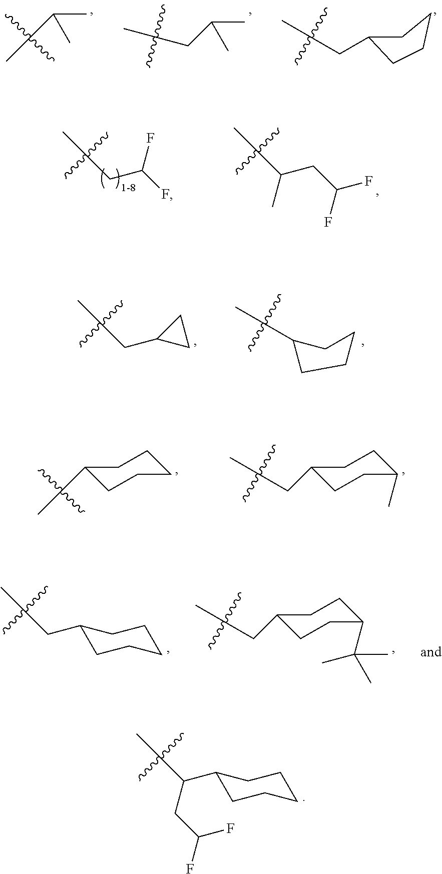

In some embodiments, at least one R⁶ is

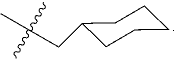 [SMILES: *CC1CCCCC1]

In some embodiments, at least one R⁶ is

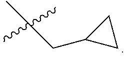 [SMILES: *CC1CC1]

In some embodiments, each R⁶ is chosen from

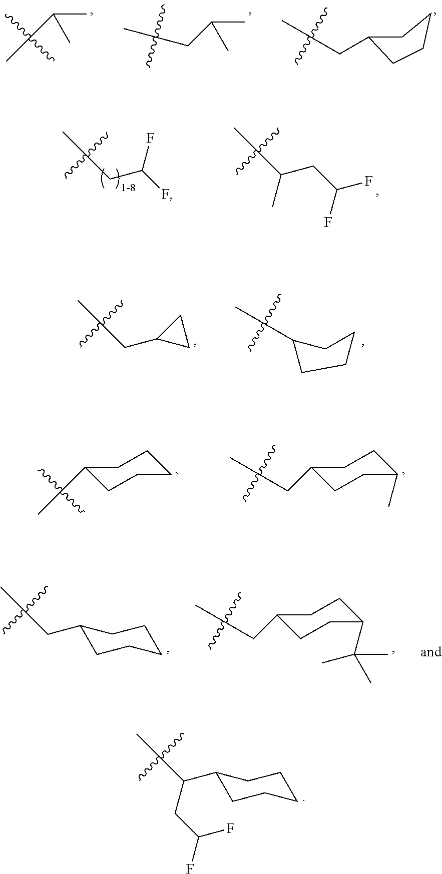

In some embodiments, each R⁶ is

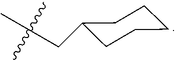 [SMILES: *CC1CCCCC1]

In some embodiments, each R⁶ is

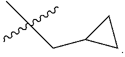 [SMILES: *CC1CC1]

In some embodiments, at least one R⁷ is —OH. In some embodiments, at least one R⁷ is chosen from —NHY³ groups. In some embodiments, at least one R⁷ is chosen from —NY³Y⁴ groups. In some embodiments, each R⁷, which may be identical or different, is independently chosen from —NHY³ groups. In some embodiments, each R⁷, which may be identical or different, is independently chosen from —NY³Y⁴ groups. In some embodiments, each R⁷ is identical and chosen from —NHY³ groups. In some embodiments, each R⁷ is identical and chosen from

—NY³Y⁴ groups. In some embodiments, each R⁷ is —OH.

In some embodiments, at least one Y³ and/or at least one Y⁴ is chosen from C₁₋₈ alkyl and C₁₋₈ haloalkyl groups. In some embodiments, at least one Y³ and/or at least one Y⁴ is chosen from C₁₋₈ alkyl groups. In some embodiments, at least one Y³ and/or at least one Y⁴ is chosen from C₁₋₈ haloalkyl groups. In some embodiments, each Y³ and/or each Y⁴, which may be identical or different, are independently chosen from C₁₋₈ alkyl and C₁₋₈ haloalkyl groups. In some embodiments, each Y³ and/or each Y⁴, which may be identical or different, are independently chosen from C₁₋₈ alkyl groups. In some embodiments, each Y³ and/or each Y⁴, which may be identical or different, are independently chosen from C₁₋₈ haloalkyl groups.

In some embodiments, each Y³ is identical and chosen from C₁₋₈ alkyl and C₁₋₈ haloalkyl groups. In some embodiments, each Y³ is identical and chosen from C₁₋₈ alkyl groups. In some embodiments, each Y³ is identical and chosen from C₁₋₈ haloalkyl groups.

In some embodiments, each Y⁴ is identical and chosen from C₁₋₈ alkyl and C₁₋₈ haloalkyl groups. In some embodiments, each Y⁴ is identical and chosen from C₁₋₈ alkyl groups. In some embodiments, each Y⁴ is identical and chosen from C₁₋₈ haloalkyl groups.

In some embodiments, at least one Y³ and/or at least one Y⁴ is methyl. In some embodiments, at least one Y³ and/or at least one Y⁴ is ethyl. In some embodiments, at least one Y³ and/or at least one Y⁴ is H. In some embodiments, each Y³ and/or each Y⁴ is methyl. In some embodiments, each Y³ and/or each Y⁴ is ethyl. In some embodiments, each Y³ and/or each Y⁴ is H.

In some embodiments, at least one Y² and at least one Y³ join together along with the nitrogen atom to which they are attached to form a ring. In some embodiments, each Y² and each Y³ join together along with the nitrogen atom to which they are attached to form a ring.

In some embodiments, at least one R⁷ is chosen from

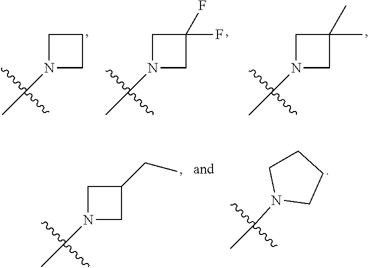

In some embodiments, each R⁷ is

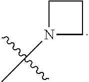 [SMILES: *N1CCC1]

In some embodiments, each R⁷ is

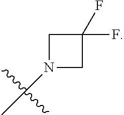 [SMILES: *N1CC(F)(F)C1]

In some embodiments, each R⁷ is

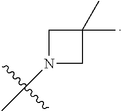 [SMILES: *N1CC(C)(C)C1]

In some embodiments, each R⁷ is

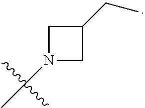 [SMILES: *N1CC(CC)C1]

In some embodiments, each R⁷ is

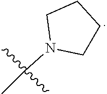 [SMILES: *N1CCCC1]

In some embodiments, at least one R⁴ is chosen from halomethyl groups. In some embodiments, at least one R⁴ is CF₃. In some embodiments, at least one R⁴ is CH₃. In some embodiments, at least one R⁴ is CN. In some embodiments, each R⁴, which may be identical or different, is independently chosen from halomethyl groups. In some embodiments, each R⁴ is identical and chosen from halomethyl groups. In some embodiments, each R⁴ is CF₃. In some embodiments, each R⁴ is CH₃. In some embodiments, each R⁴ is CN.

In some embodiments, m is chosen from integers ranging from 2 to 128. In some embodiments, m is chosen from integers ranging from 2 to 64. In some embodiments, m is chosen from integers ranging from 2 to 32. In some embodiments, m is chosen from integers ranging from 2 to 16. In some embodiments, m is chosen from integers ranging from 2 to 8. In some embodiments, m is chosen from integers ranging from 2 to 4. In some embodiments, m is 4. In some embodiments, m is 3. In some embodiments, m is 2.

In some embodiments, linker groups may be chosen from groups comprising spacer groups, such spacer groups as, for example, —(CH₂)ₚ— and —O(CH₂)ₚ—, wherein p is chosen from integers ranging from 1 to 250. Other non-limiting examples of spacer groups include carbonyl groups and carbonyl-containing groups such as, for example, amide groups. A non-limiting example of a spacer group is

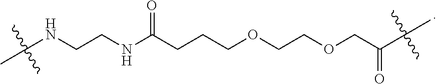 [SMILES: *NCCNC(=O)CCCOCCOCC(*)=O]

In some embodiments, the linker group is chosen from

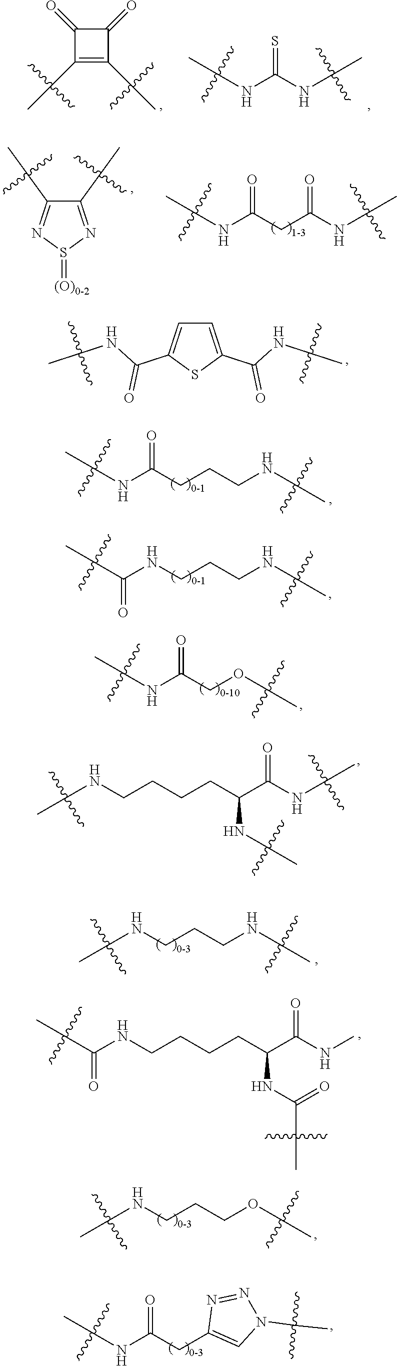

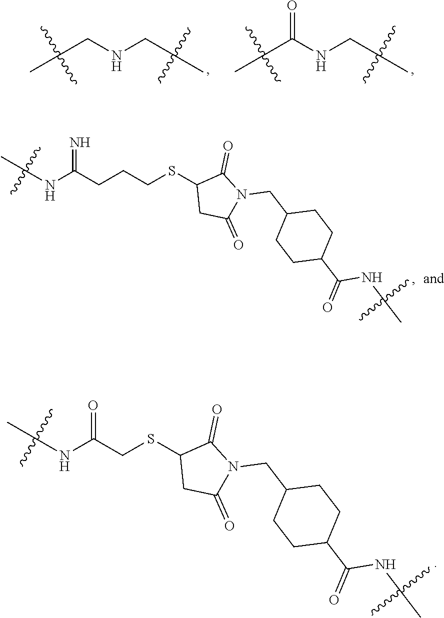 [SMILES: *CNC* ; *CNC(*)=O ; *NC(=N)CCCSC1CC(=O)N(CC2CCC(C(=O)N*)CC2)C1=O ; *NC(=O)CSC1CC(=O)N(CC2CCC(C(=O)N*)CC2)C1=O]

Other linker groups, such as, for example, polyethylene glycols (PEGs) and —C(═O)—NH—(CH₂)ₚ—C(═O)—NH—, wherein p is chosen from integers ranging from 1 to 250, will be familiar to those of ordinary skill in the art and/or those in possession of the present disclosure.

In some embodiments, the linker group is

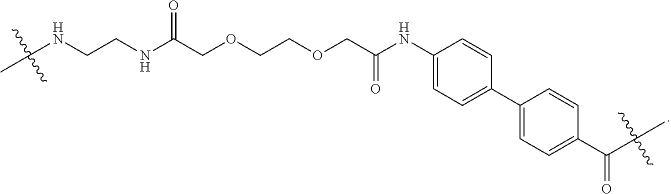 [SMILES: *NCCNC(=O)COCCOCC(=O)Nc1ccc(-c2ccc(C(*)=O)cc2)cc1]

In some embodiments, the linker group is

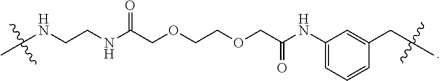 [SMILES: *Cc1cccc(NC(=O)COCCOCC(=O)NCCN*)c1]

In some embodiments, the linker group is chosen from —C(═O)NH(CH₂)₂NH—, —CH₂NHCH₂—, and —C(═O)NHCH₂—. In some embodiments, the linker group is —C(═O)NH(CH₂)₂NH—.

In some embodiments, L is chosen from dendrimers. In some embodiments, L is chosen from polyamidoamine (“PAMAM”) dendrimers. In some embodiments, L is chosen from PAMAM dendrimers comprising succinamic. In some embodiments, L is PAMAM GO generating a tetramer. In some embodiments, L is PAMAM G1 generating an octamer. In some embodiments, L is PAMAM G2 generating a 16-mer. In some embodiments, L is PAMAM G3 generating a 32-mer. In some embodiments, L is PAMAM G4 generating a 64-mer. In some embodiments, L is PAMAM G5 generating a 128-mer.

In some embodiments, L is chosen from

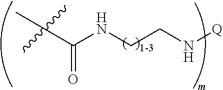

wherein Q is a chosen from

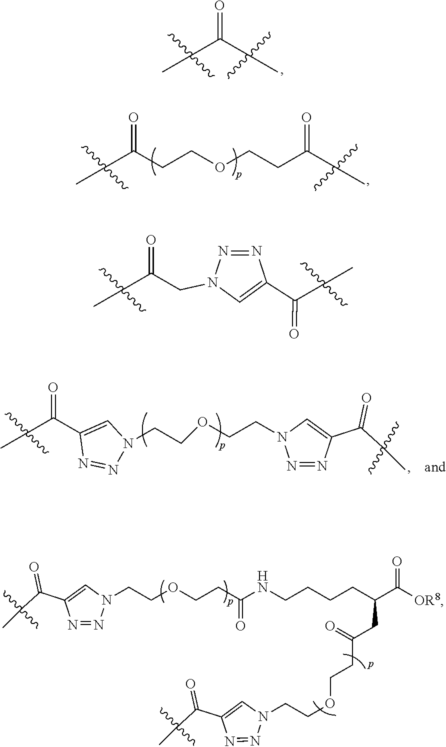

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250. In some embodiments, R⁸ is chosen from C₁₋₈ alkyl. In some embodiments, R⁸ is chosen from C₇₋₁₉ arylalkyl. In some embodiments, R⁸ is H. In some embodiments, R⁸ is benzyl.

In some embodiments, L is chosen from

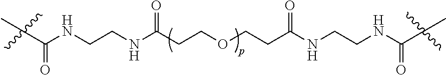

wherein p is chosen from integers ranging from 0 to 250.

In some embodiments, L is chosen from

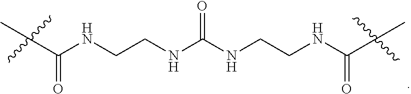 [SMILES: *C(=O)NCCNC(=O)NCCNC(*)=O]

In some embodiments, L is chosen from

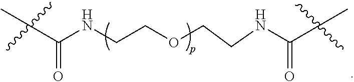

wherein p is chosen from integers ranging from 0 to 250.

In some embodiments, L is chosen from

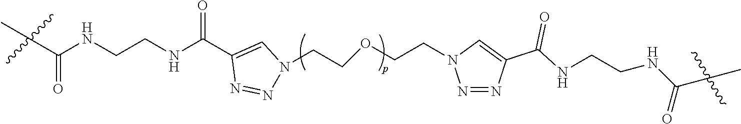

wherein p is chosen from integers ranging from 0 to 250.

In some embodiments, L is chosen from

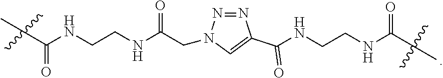 [SMILES: *C(=O)NCCNC(=O)Cn1cc(C(=O)NCCNC(*)=O)nn1]

In some embodiments, L is chosen from

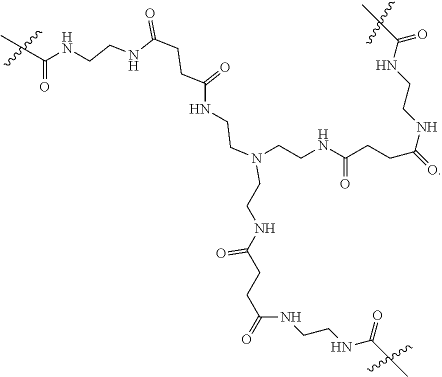 [SMILES: *C(=O)NCCNC(=O)CCC(=O)NCCN(CCNC(=O)CCC(=O)NCCNC(*)=O)CCNC(=O)CCC(=O)NCCNC(*)=O]

In some embodiments, L is chosen from

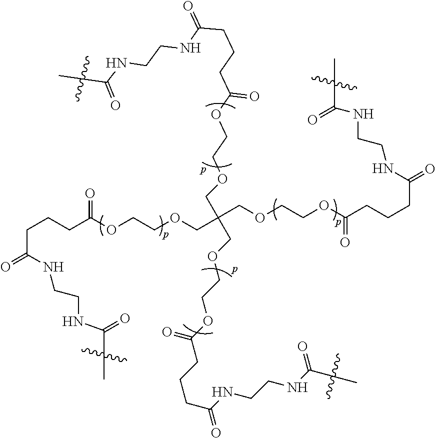

wherein p is chosen from integers ranging from 0 to 250.

In some embodiments, L is chosen from

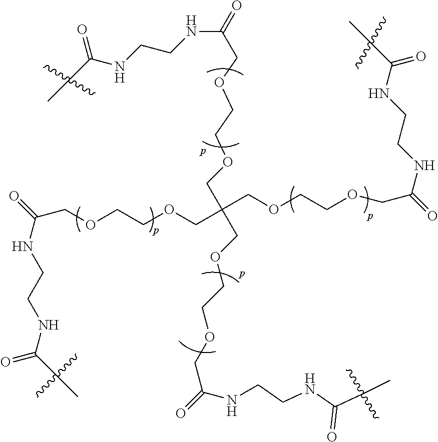

wherein p is chosen from integers ranging from 0 to 250.

In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 30. In some embodiments, p is chosen from integers ranging from 0 to 15. In some embodiments, p is chosen from integers ranging from 0 to 10. In some embodiments, p is chosen from integers ranging from 0 to 5. In some embodiments, p is 117. In some embodiments, p is 25. In some embodiments, p is 21. In some embodiments, p is 17. In some embodiments p is 13. In some embodiments, p is 10. In some embodiments, p is 8. In some embodiments, p is 6. In some embodiments, p is 5. In some embodiments, p is 4. In some embodiments, p is 3. In some embodiments, p is 2. In some embodiments, p is 1. In some embodiments, p is 0.

In some embodiments, the at least one compound is chosen from compounds of Formula (I), wherein said compound is symmetrical.

In some embodiments, the at least one compound is chosen from compounds having the following Formula:

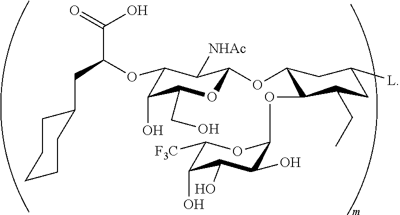

In some embodiments, the at least one compound is chosen from compounds having the following Formula:

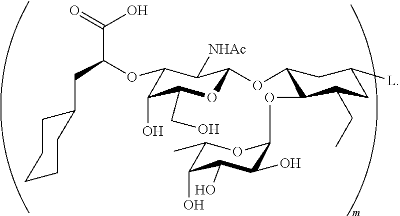

In some embodiments, the at least one compound is chosen from compounds having the following Formula:

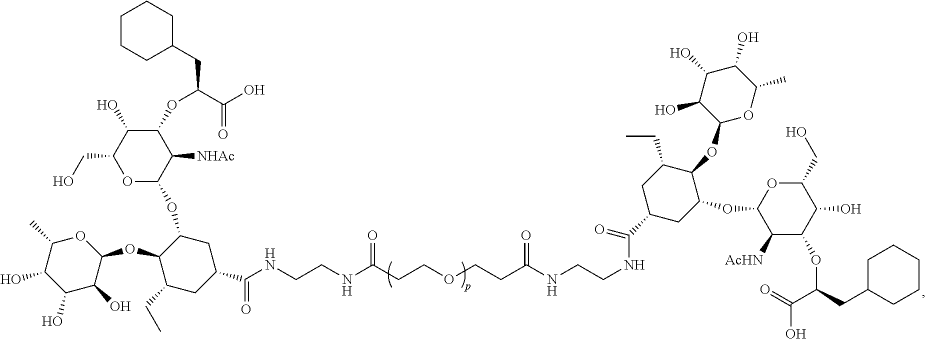

wherein p is chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10.

In some embodiments, the at least one compound is chosen from compounds having the following Formulae:

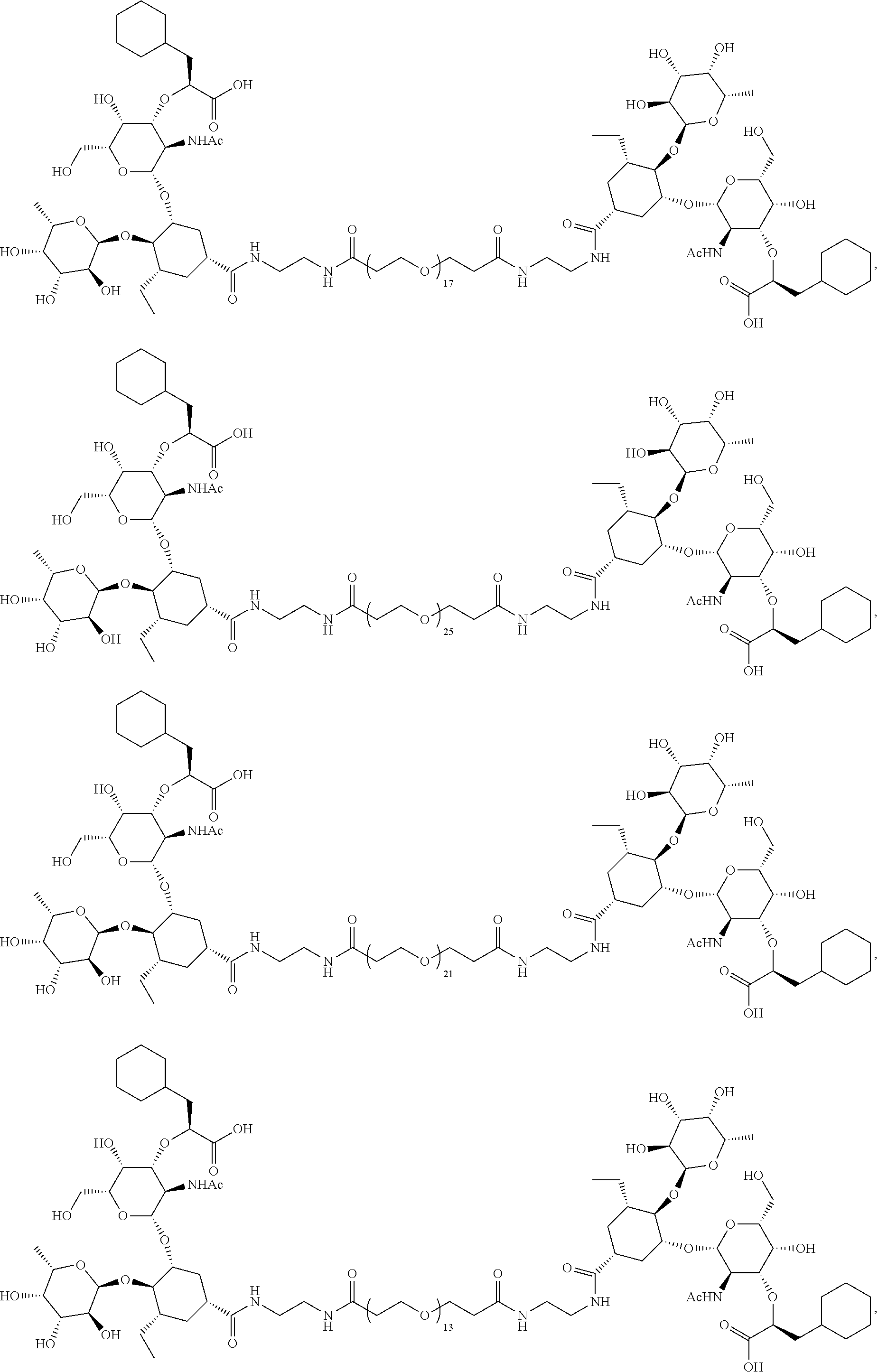

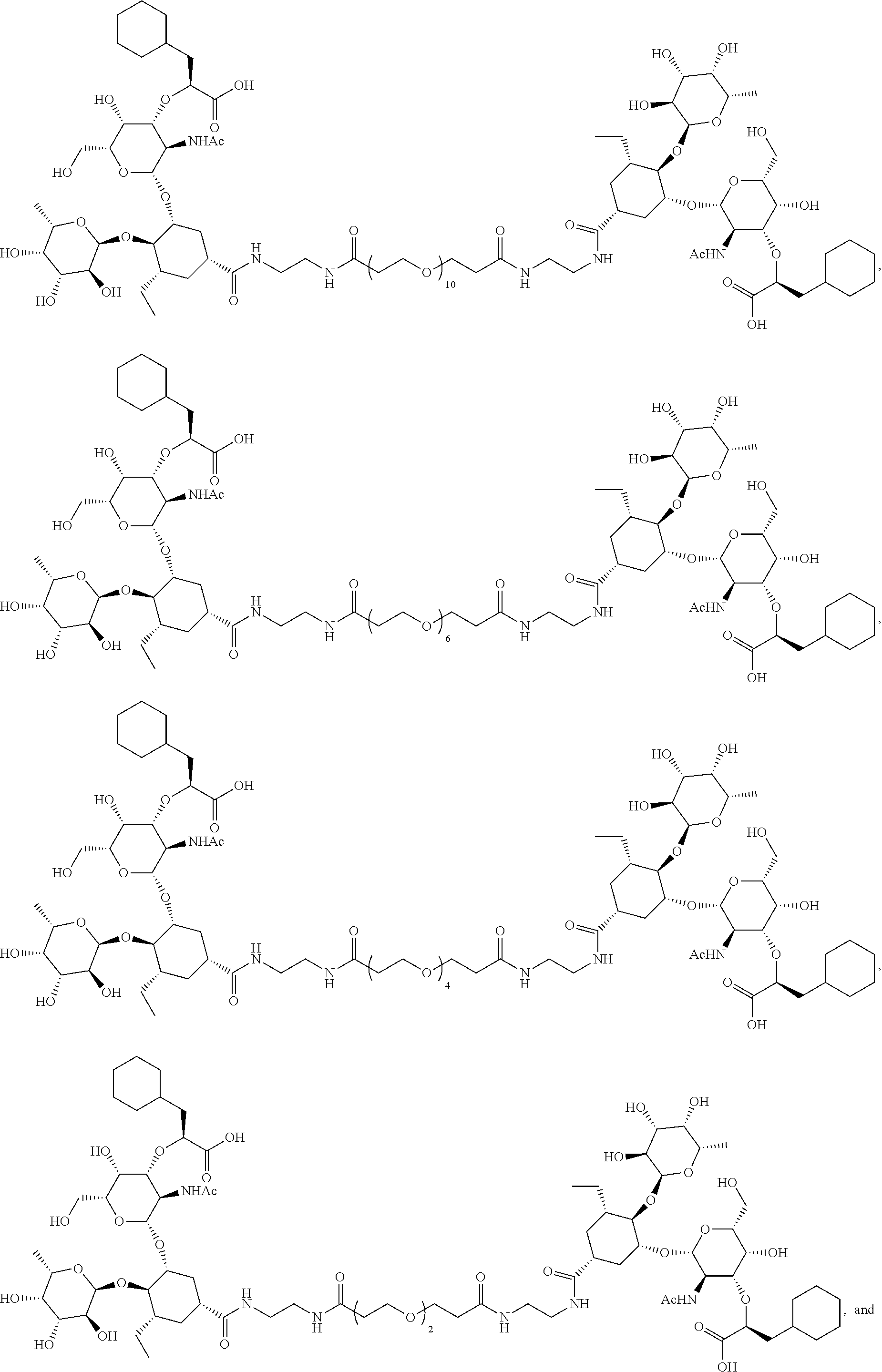

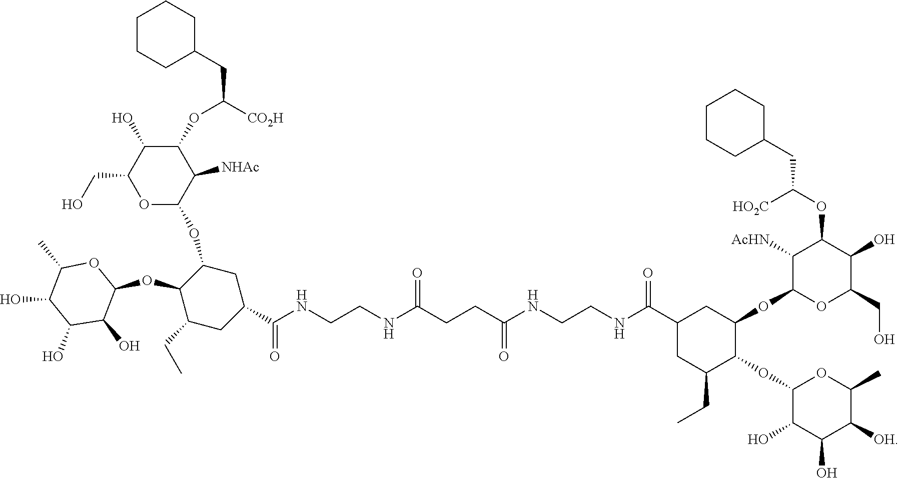 [SMILES: CC[C@H]1CC(C(=O)NCCNC(=O)CCC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)O)[C@H]3NC(C)=O)C2)C[C@@H](O[C@@H]2O[C@H](CO)[C@H](O)[C@H](O[C@@H](CC3CCCCC3)C(=O)O)[C@H]2NC(C)=O)[C@@H]1O[C@@H]1O[C@@H](C)[C@@H](O)[C@@H](O)[C@@H]1O]

In some embodiments, the at least one compound is chosen from compounds having the following Formula:

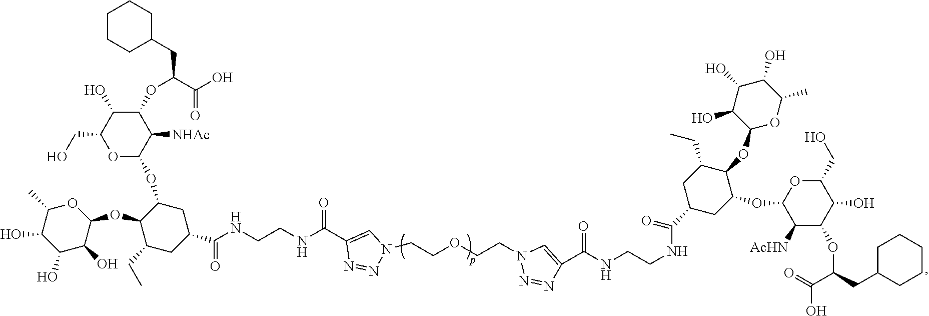

wherein p is chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10. In some embodiments, p is chosen from integers ranging from 0 to 5.

In some embodiments, the at least one compound is chosen from compounds having the following Formulae:

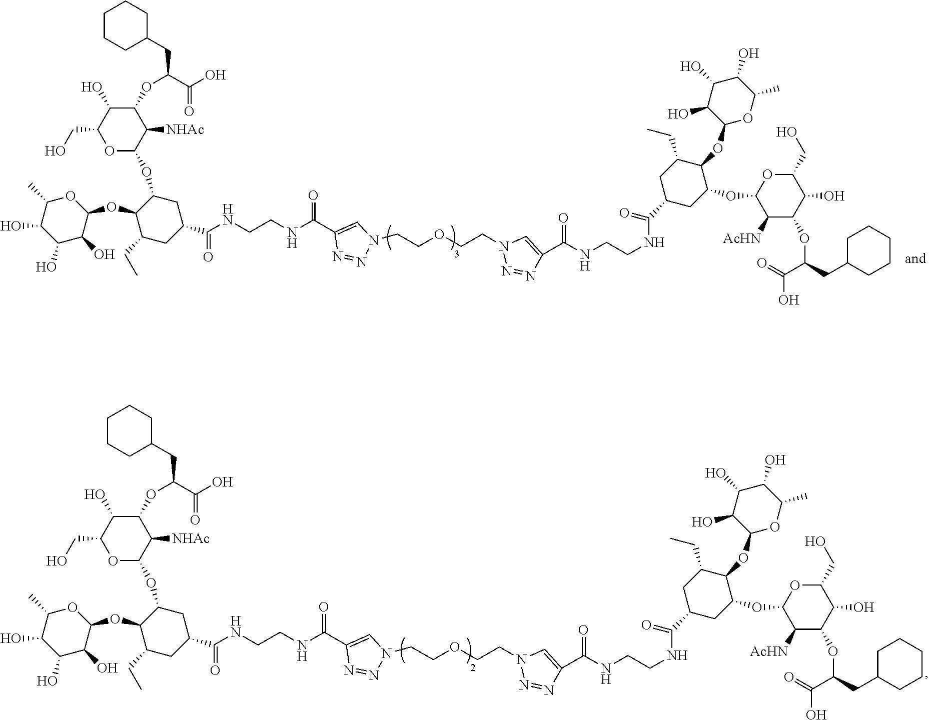

In some embodiments, the at least one compound is chosen from compounds having the following Formula:

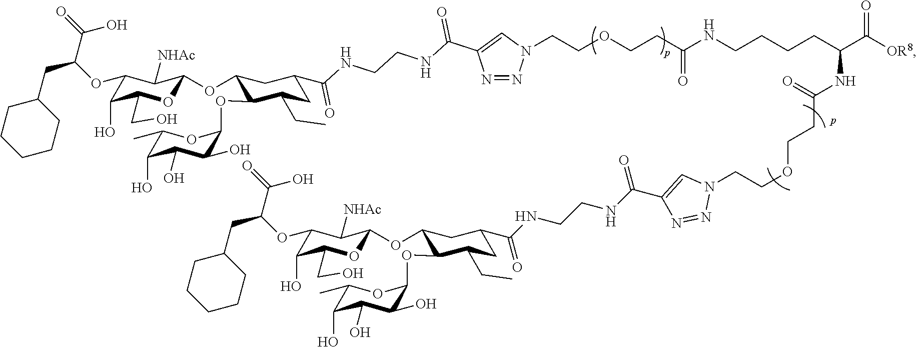

wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250. In some embodiments, R⁸ is chosen from H, C₁₋₈ alkyl, and C₇₋₁₉ arylalkyl groups. In some embodiments, R⁸ is chosen from C₁₋₈ alkyl groups. In some embodiments, R⁸ is chosen from C₇₋₁₉ arylalkyl groups. In some embodiments, R⁸ is H. In some embodiments, R⁸ is benzyl. In some embodiments, each p is identical and chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10. In some embodiments, p is chosen from integers ranging from 0 to 5.

In some embodiments, the at least one compound is chosen from compounds having the following Formulae:

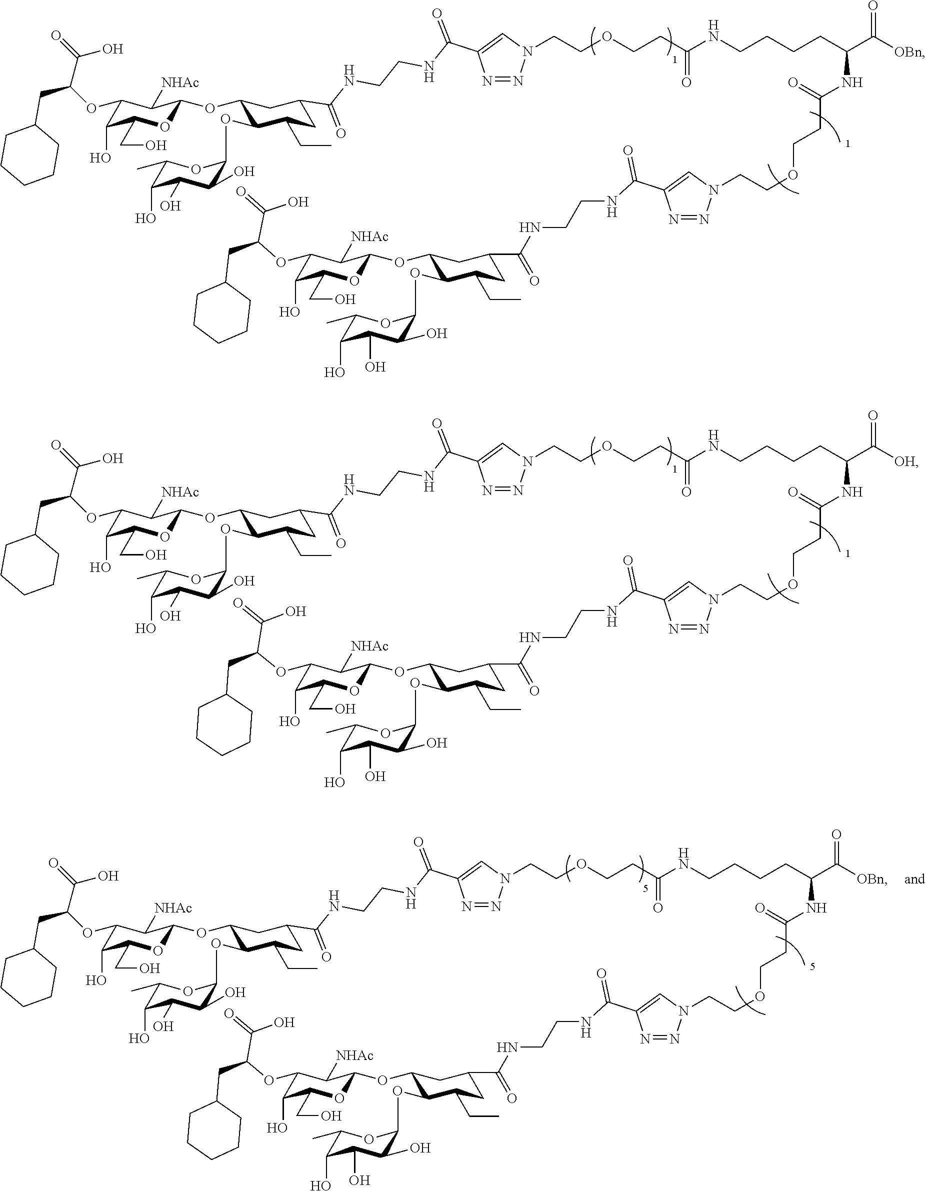

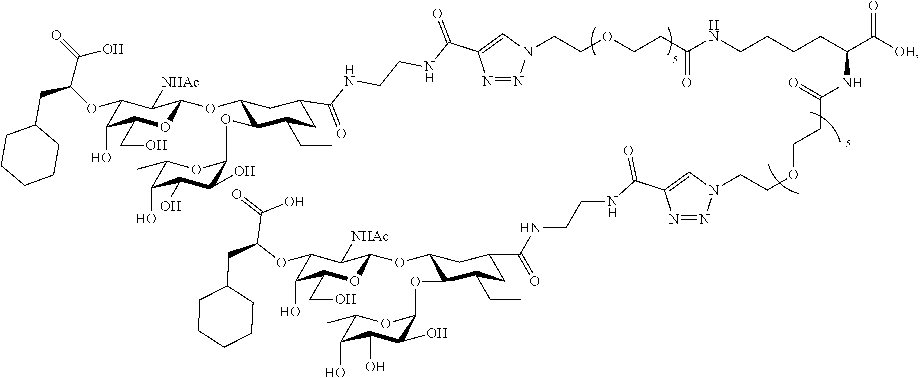

In some embodiments, the at least one compound is chosen from compounds having the following Formula:

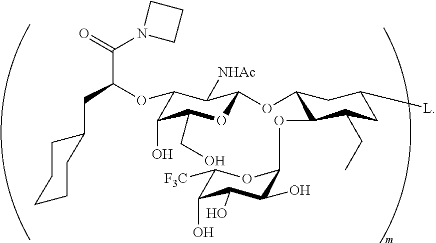

In some embodiments, the at least one compound is chosen from compounds having the following Formula:

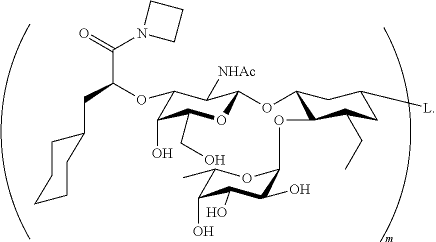

In some embodiments, the at least one compound is chosen from compounds having the following Formula:

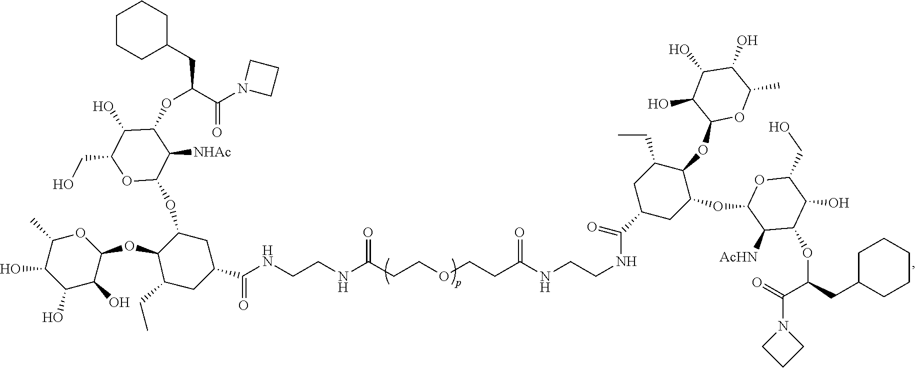

wherein p is chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10.

In some embodiments, the at least one compound is chosen from compounds having the following Formulae:

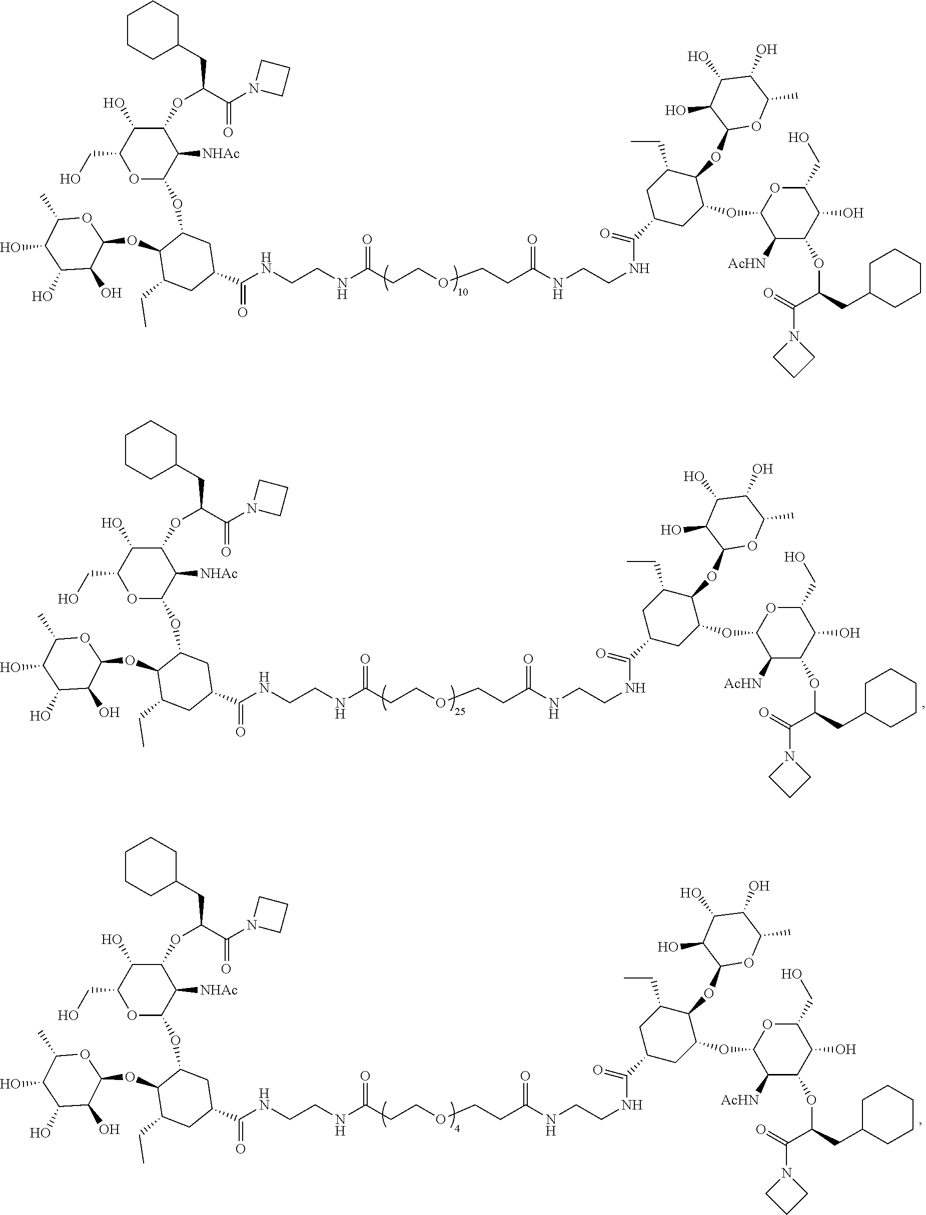

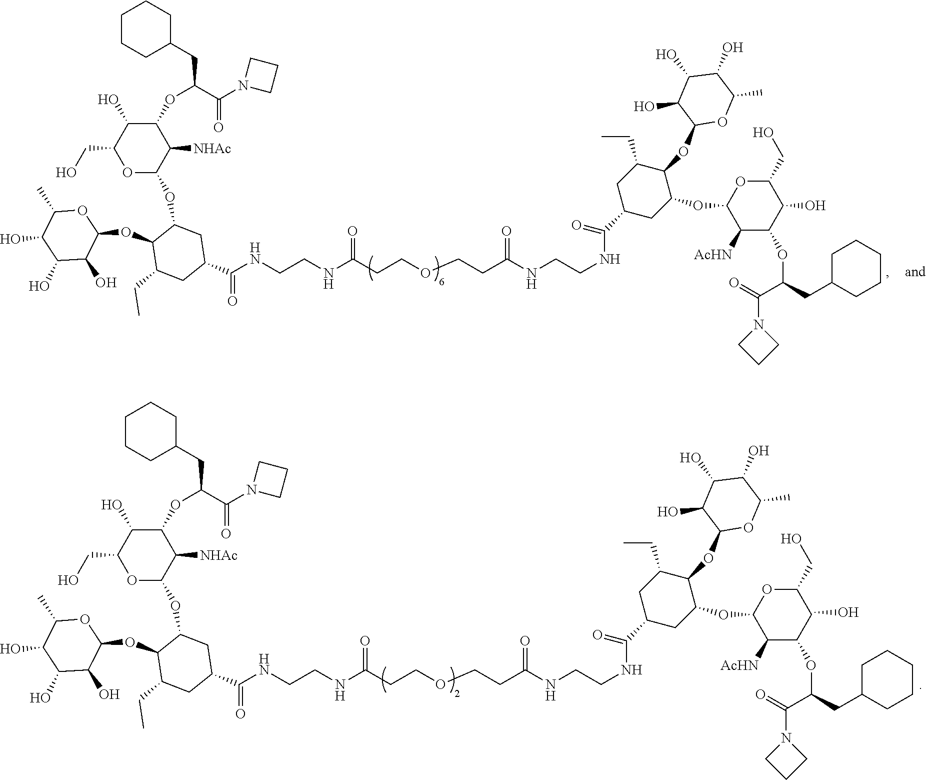

In some embodiments, the at least one compound is chosen from compounds having the following Formula:

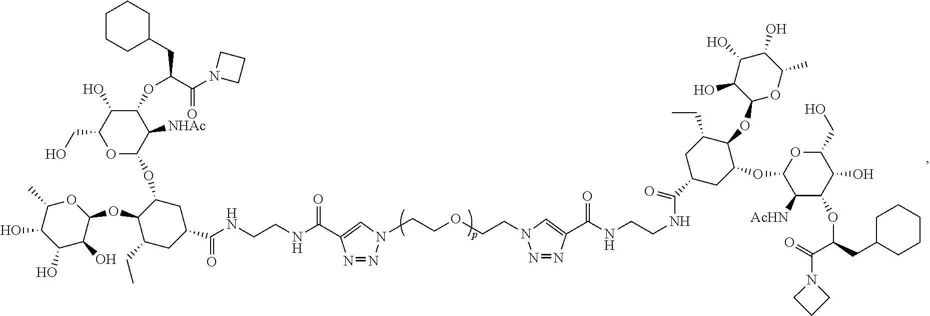

wherein p is chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10.

In some embodiments, the at least one compound is chosen from compounds having the following Formulae:

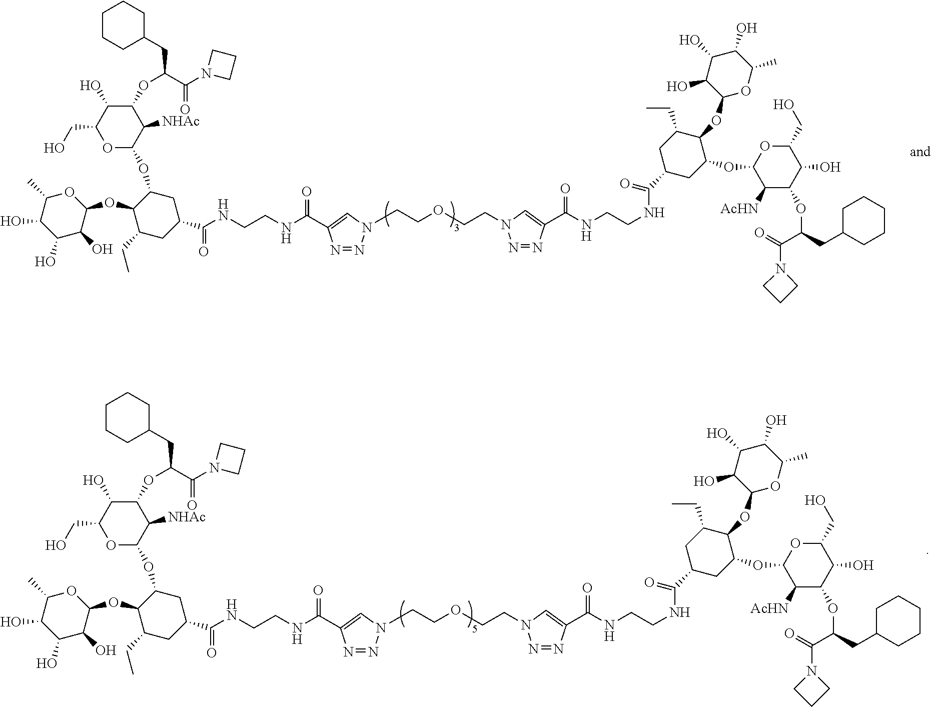

In some embodiments, the at least one compound is chosen from compounds having the following Formula:

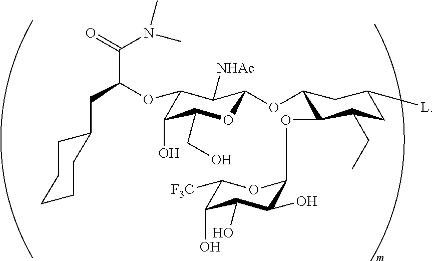

In some embodiments, the at least one compound is chosen from compounds having the following Formula:

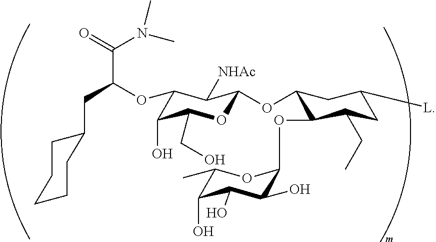

In some embodiments, the at least one compound is chosen from compounds having the following Formula:

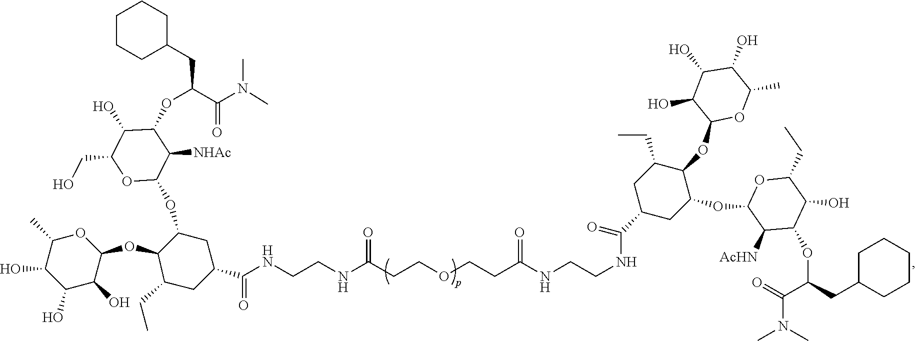

wherein p is chosen from integers ranging from 0 to 250. In some embodiments, p is chosen from integers ranging from 0 to 200. In some embodiments, p is chosen from integers ranging from 0 to 150. In some embodiments, p is chosen from integers ranging from 0 to 100. In some embodiments, p is chosen from integers ranging from 0 to 50. In some embodiments, p is chosen from integers ranging from 0 to 25. In some embodiments, p is chosen from integers ranging from 0 to 13. In some embodiments, p is chosen from integers ranging from 0 to 10.

In some embodiments, the at least one compound is:

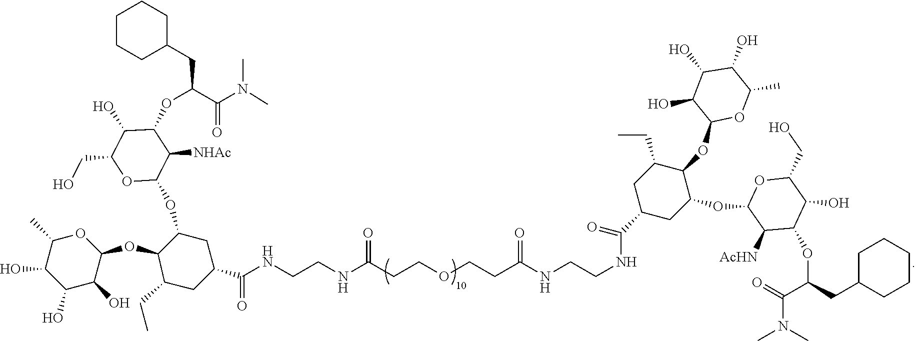

In some embodiments, the at least one compound is chosen from compounds of the following Formulae:

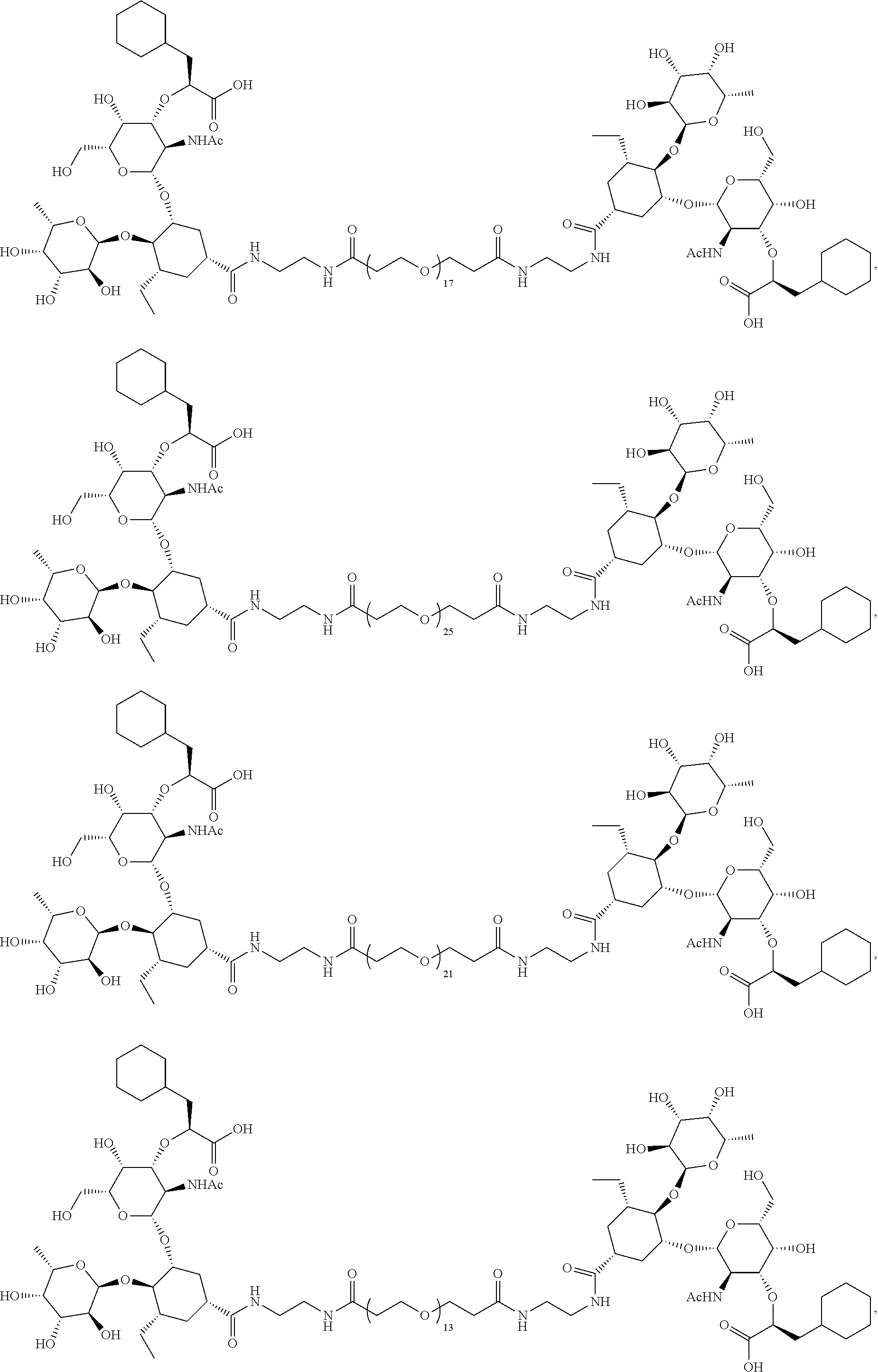

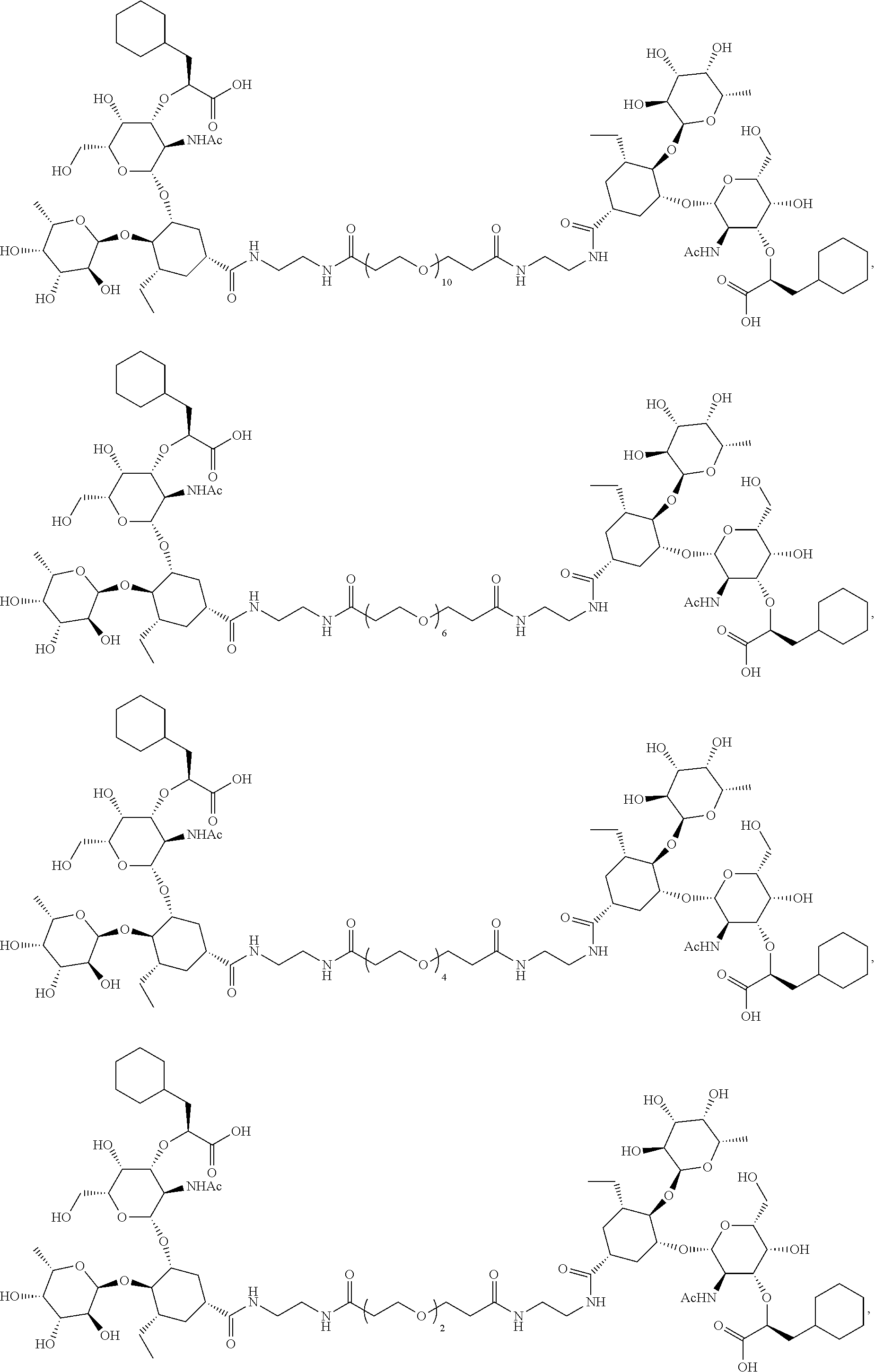

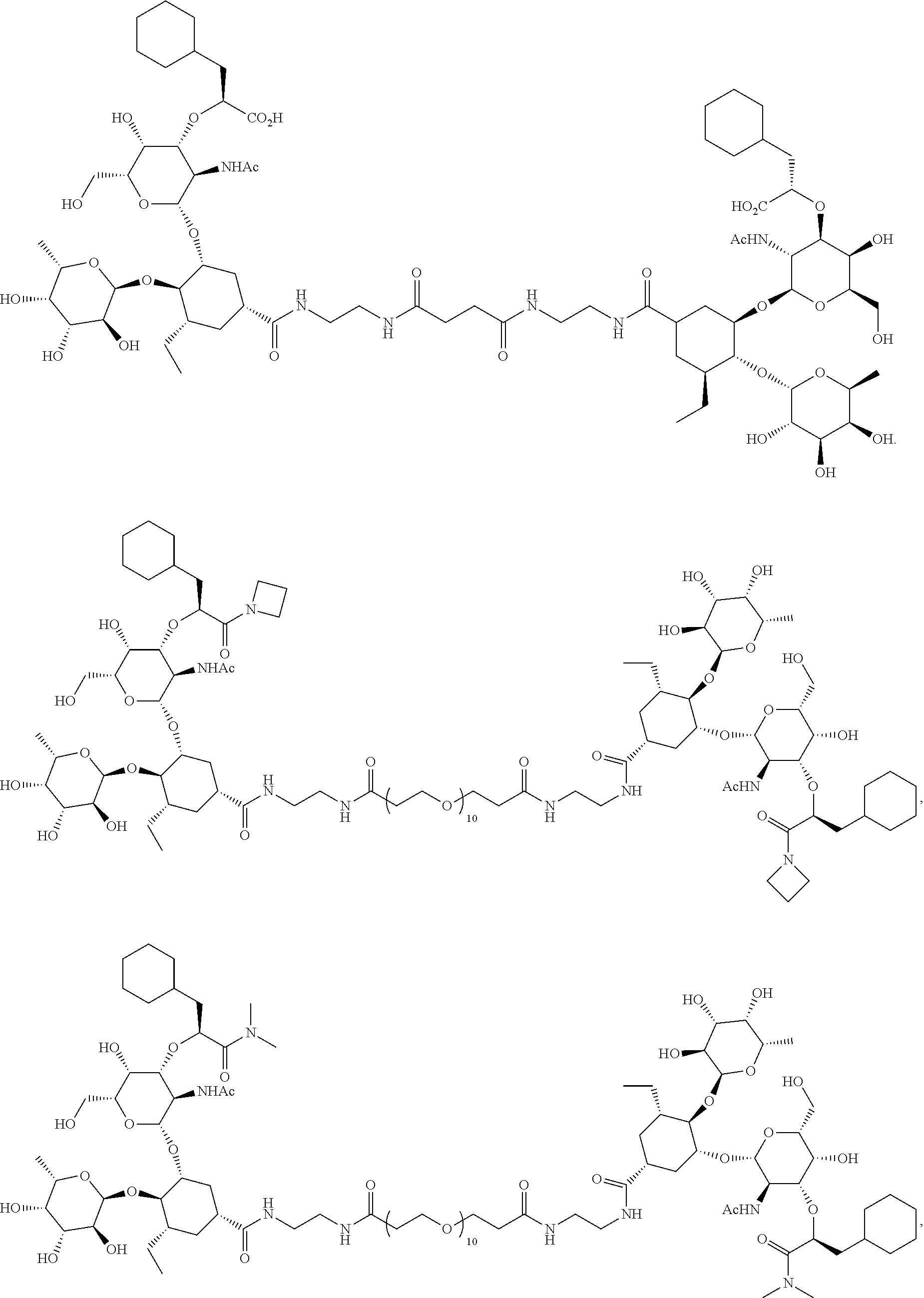

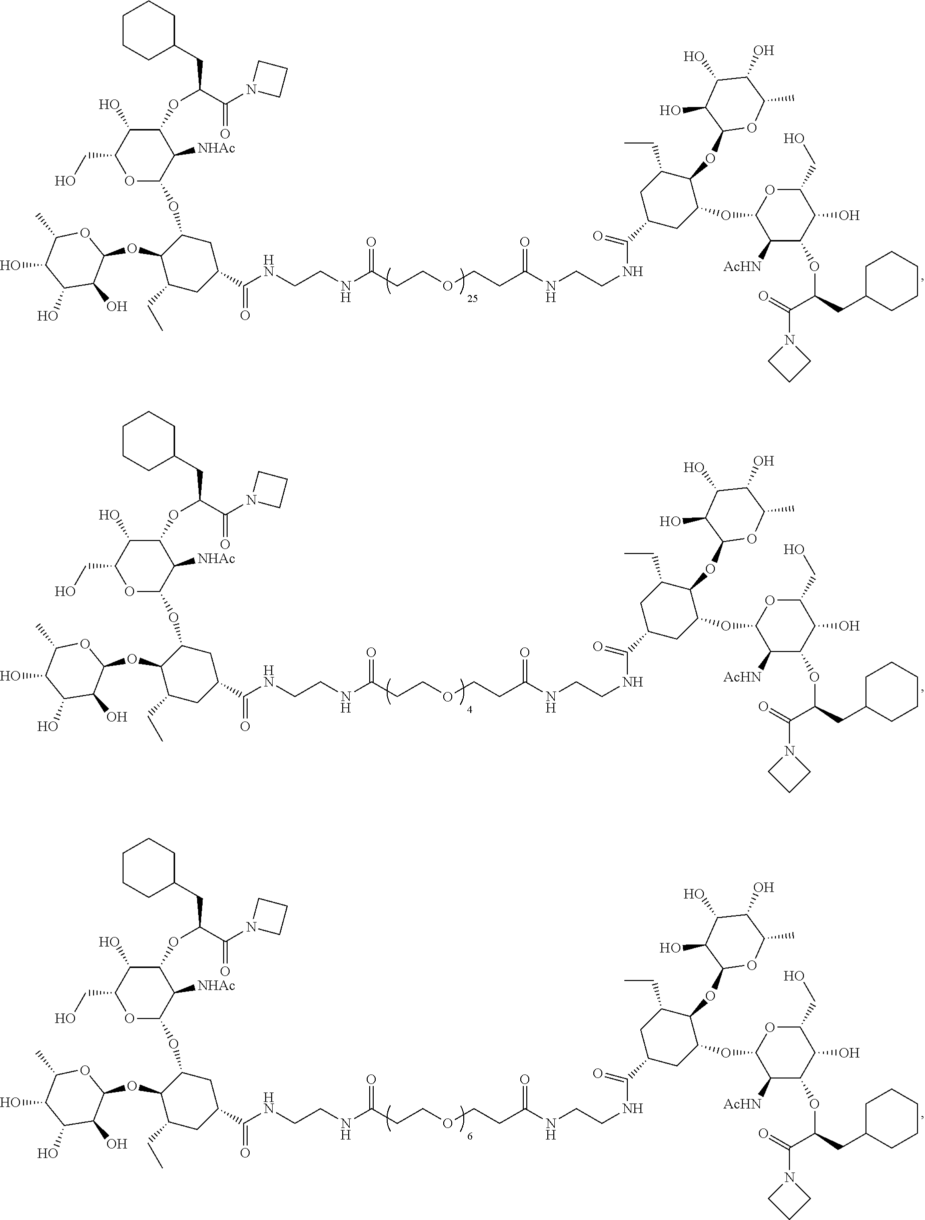

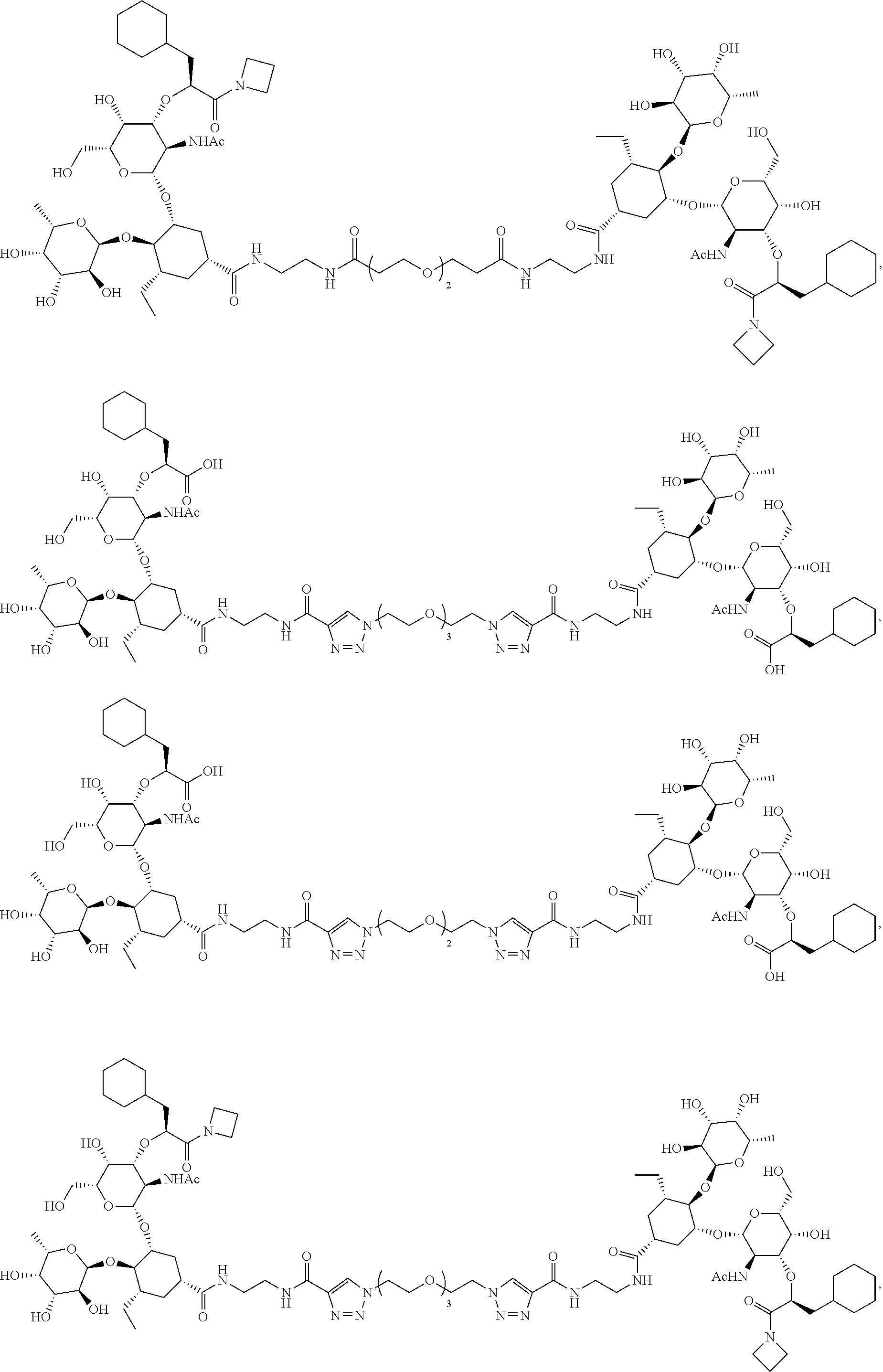

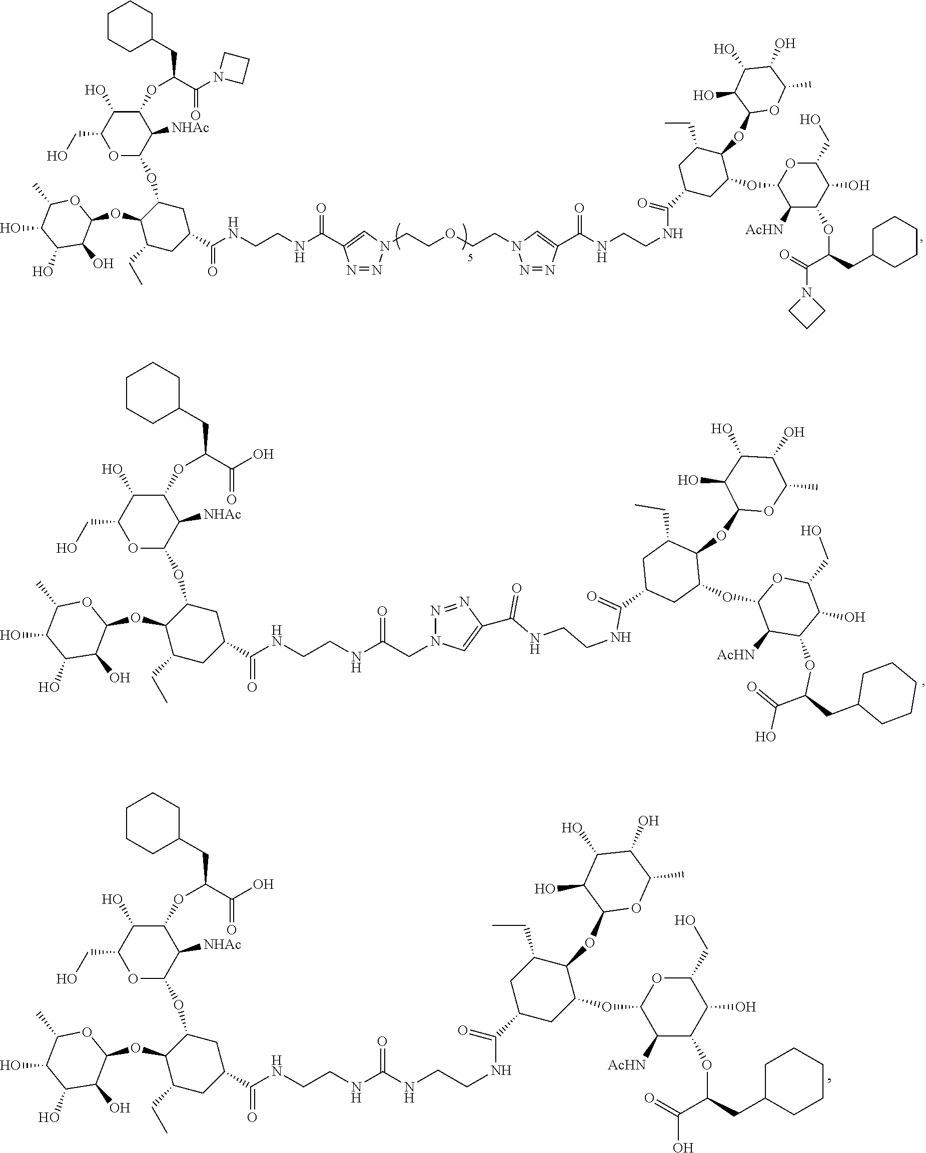

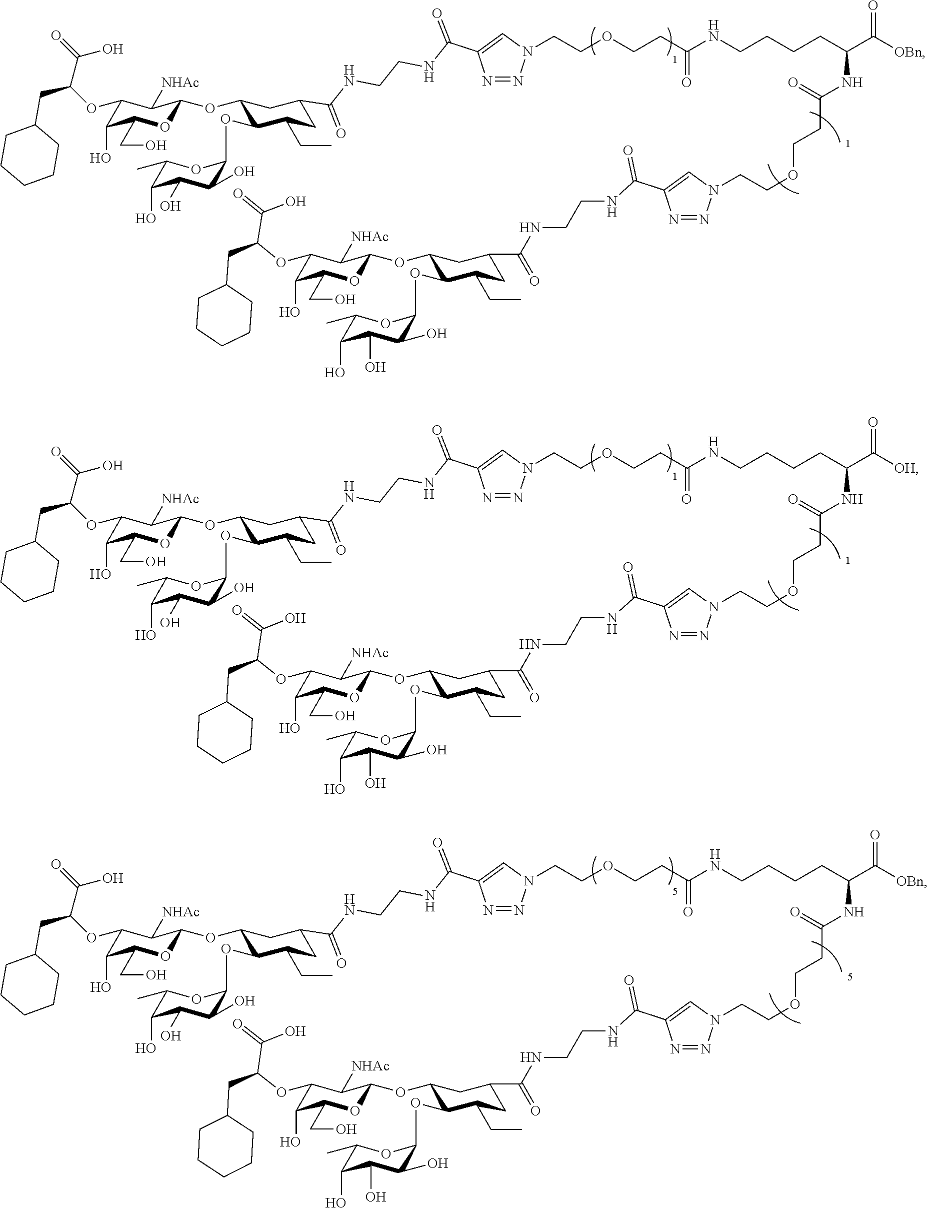

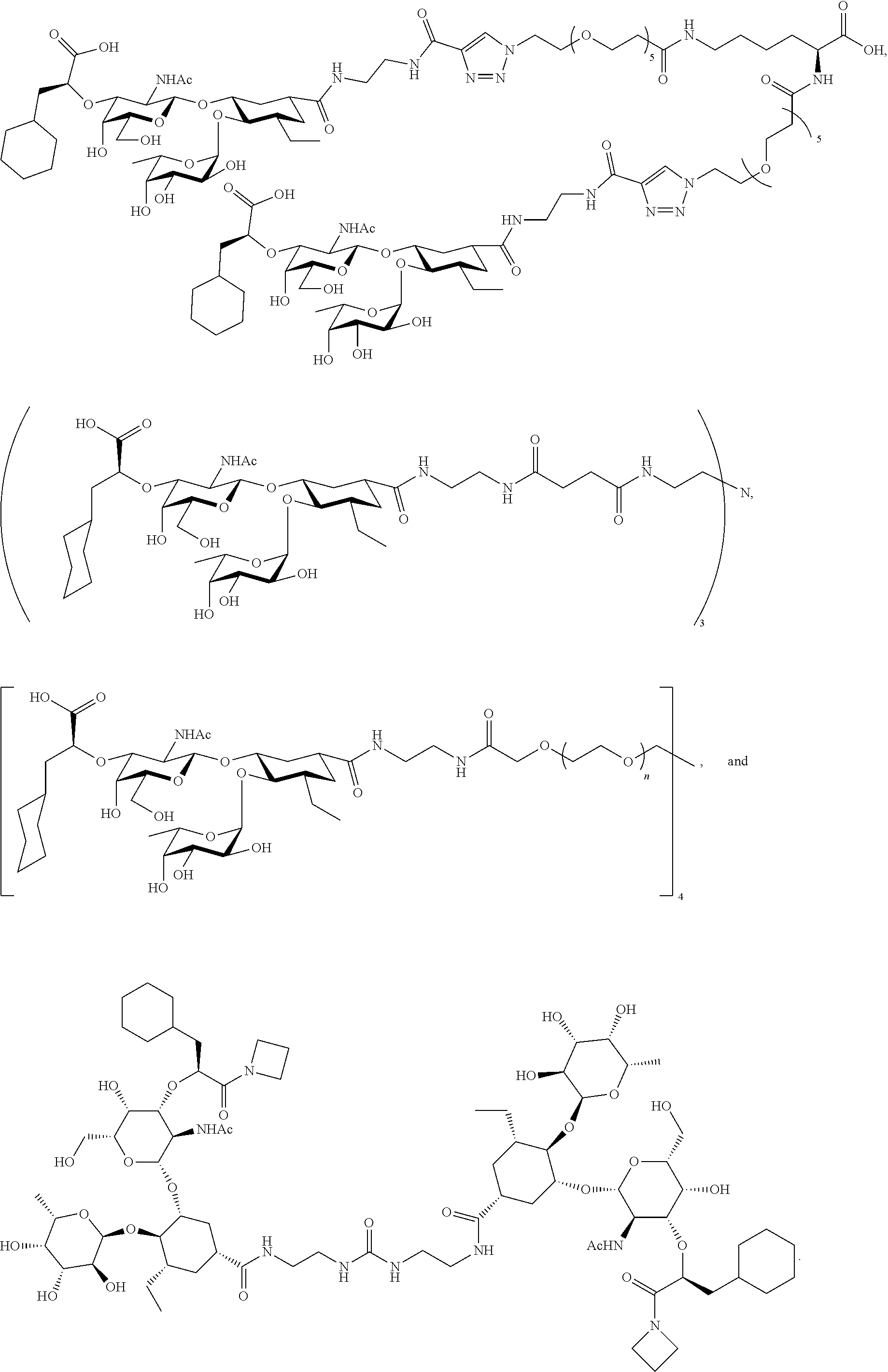

Also provided are methods for the treatment of sickle cell disease or complications associated therewith, including, for example, vaso-occlusion crises, the methods comprising administering to a subject in need thereof pharmaceutical compositions comprising at least one compound of Formula (I). Such pharmaceutical compositions are described in greater detail herein.

In some embodiments, a method for the treatment of sickle cell disease or complications associated therewith where inhibition of E-selectin mediated functions may be useful is disclosed, the method comprising administering to a subject in need thereof an effective amount of at least one compound of Formula (I) and/or a pharmaceutical composition comprising at least one compound of Formula (I).

In some embodiments, a method for the treatment of vaso-occlusion crises where inhibition of E-selectin mediated functions may be useful is disclosed, the method comprising administering to a subject in need thereof an effective amount of at least one compound of Formula (I) and/or a pharmaceutical composition comprising at least one compound of Formula (I).

In some embodiments, a compound of Formula (I) and/or a pharmaceutical composition comprising at least one compound of Formula (I) may be used for the preparation and/or manufacture of a medicament for use in the treatment of sickle cell disease or complications associated therewith, including, for example, vaso-occlusive crisis.

Whenever a term in the specification is identified as a range (e.g., C₁₋₄ alkyl), the range independently discloses and includes each element of the range. As a non-limiting example, C₁₋₄ alkyl groups includes, independently, C₁ alkyl groups, C₂ alkyl groups, C₃ alkyl groups, and C₄ alkyl groups.

The term “at least one” refers to one or more, such as one, two, etc. For example, the term “at least one C₁₋₄ alkyl group” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The term “E-selectin antagonist” includes inhibitors of E-selectin only, as well as inhibitors of E-selectin and either P-selectin or L-selectin, and inhibitors of E-selectin, P-selectin, and L-selectin.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized; and the heterocyclyl group Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof.

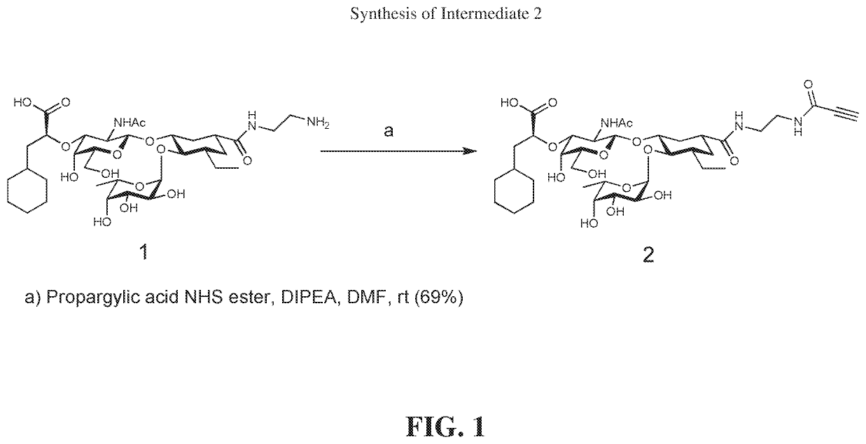

Compounds of Formula (I) may be prepared according to the General Reaction Scheme shown in FIG. 1. It is understood that one of ordinary skill in the art may be able to make these compounds by similar methods or by combining other methods known to one of ordinary skill in the art. It is also understood that one of ordinary skill in the art would be able to make, in a similar manner as described in FIG. 1, other compounds of Formula (I) not specifically illustrated herein by using appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell-based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g., a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

The terms “treat” and “treatment” include medical management of a disease, disorder, and/or condition of a subject (i.e., patient, individual) as would be understood by a person of ordinary skill in the art (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

In some embodiments of the methods described herein, the subject is a human. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating diseases, disorders, and/or conditions treatable by inhibiting an activity of E-selectin can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Also provided herein are methods for the treatment of sickle cell disease or complications associated therewith, or vaso-occlusive crisis, the methods comprising administering to a subject in need thereof pharmaceutical compositions comprising an effective amount of at least one compound of Formula (I). In some embodiments, the pharmaceutical composition administered further comprises at least one additional pharmaceutically acceptable ingredient.

In pharmaceutical dosage forms, any one or more of the compounds of the present disclosure may be administered in the form of a pharmaceutically acceptable derivative, such as a salt, and/or it or they may also be used alone and/or in appropriate association, as well as in combination, with other pharmaceutically active compounds.

An effective amount or therapeutically effective amount refers to an amount of at least one compound of the present disclosure or a pharmaceutical composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.01 μg to about 3000 μg per kg weight of the subject. The minimum dose that is sufficient to provide effective therapy may be used in some embodiments. Subjects may generally be monitored for therapeutic effectiveness using assays suitable for the disease, disorder and/or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may, for example, be sterile aqueous or sterile non-aqueous solutions, suspensions, or emulsions, and may additionally comprise at least one pharmaceutically acceptable excipient (i.e., a non-toxic material that does not interfere with the activity of the active ingredient). Such compositions may, for example, be in the form of a solid, liquid, or gas (aerosol). Alternatively, the compositions described herein may, for example, be formulated as a lyophilizate, or compounds described herein may be encapsulated within liposomes using technology known in the art. The pharmaceutical compositions may further comprise at least one additional pharmaceutically acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient may be selected based on the mode of administration, as well as the chemical composition of the active ingredient(s). Compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned ingredients, excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid composition may include, for example, at least one the following: a sterile diluent such as water for injection, saline solution, including for example physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable composition, and in some embodiments, the injectable composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; the formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The pharmaceutical compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

### Example 1: Compound 11

Compound 2: A solution of 1-[(1-oxo-2-propynyl)oxy]-2,5-pyrrolidinedione (propargylic acid NHS ester) (57 mg, 0.34 mmole) in anhydrous DMF (1 mL) was added dropwise over 15 minutes to a slurry of compound 1 (0.19 g, 0.26 mmole) (preparation described in WO 2013096926) and DIPEA (0.1 mL) in anhydrous DMF (3 mL) at room temperature. The resulting solution was stirred for 1.5 hrs. The reaction mixture was concentrated under reduced pressure. The residue was separated by Combi-flash [EtOAc/(MeOH/water, 6/1, v/v), 9/1-3/7, v/v] to afford the desired compound as a light brown solid (0.14 g, 69%). MS: Calculated for C₃₇H₅₉N₃O₁₅=785.3, Found ES-positive m/z=808.3 (M+Na⁺), ES-negative m/z=784.4 (M−1).

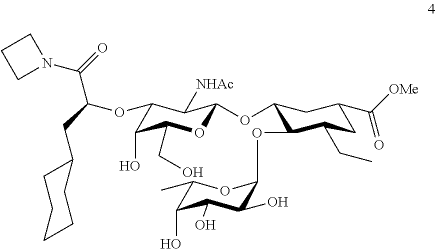

Compound 4: To a solution of compound 3 (preparation described in WO 2013096926) (2.5 g, 3.54 mmole) and DIPEA (1.2 mL, 7.08 mmole) in anhydrous DMF (15 mL) was added TBTU (1.7 g, 5.31 mmole) at 0° C. and the solution was stirred for 20 min. Azetidine (0.85 mL, 35.4 mmole) was added and the resulting solution was stirred for 1 hr while the temperature was gradually increased to room temperature. After the reaction was completed, the solution was concentrated under reduced pressure. The reaction mixture was separated by Combi-flash (EtOAc/MeOH, 4/1-2/3, v/v) to give compound 4 (1.17 g, 1.57 mmole, 44%) and lactone side product compound 5 (0.88 g, 1.28 mmole, 36%).

Compound 4: Compound 5 (0.88 g, 1.28 mmol) was dissolved in anhydrous DMF (5 mL). Azetidine (0.5 mL) was added, and then the resulting solution was stirred for 3 hrs at 50° C. The solution was concentrated and dried under high vacuum to give compound 4 (0.93 g, 1.25 mmole, 98%).

¹H NMR (400 MHz, Deuterium Oxide) δ 4.92 (d, J=4.0 Hz, 1H), 4.79 (q, J=7.3, 6.8 Hz, 1H), 4.43 (broad d, J=8.3 Hz, 1H), 4.24 (q, J=8.6 Hz, 1H), 4.15 (q, J=8.5 Hz, 1H), 4.01 (d, J=9.3 Hz, 1H), 3.99-3.80 (m, 3H), 3.76 (dd, J=10.6, 3.2 Hz, 1H), 3.73-3.51 (m, 8H), 3.42 (m, J=7.7, 4.4 Hz, 2H), 3.21 (t, J=9.7 Hz, 1H), 2.39 (broad t, J=12.7 Hz, 1H), 2.32-2.09 (m, 3H), 1.95 (s, 3H), 1.95 (m, 1H) 1.77 (m, 2H), 1.69-1.35 (m, 7H), 1.35-0.93 (m, 10H), 0.93-0.58 (m, 6H). MS: Calculated for C₃₆H₆₀N₂O₁₄=785.3, Found ES-positive m/z=767.3 (M+Na⁺), ES-negative m/z=743.4 (M−1).

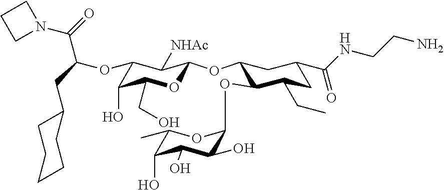

Compound 6: A solution of compound 4 (0.93 g, 1.25 mmole) in ethylene diamine (10 mL) was stirred overnight at 60° C. The solution was concentrated under reduced pressure and the residue was directly purified by silica gel column chromatography (EtOAc/MeOH, 1/2, v/v) to give compound 6 as a light yellow gel (0.9 g, 1.16 mmole, 91%) MS: Calculated for C₃₇H₆₄N₄O₁₃=772.4, Found ES-positive m/z=773.4 (M+H⁺).

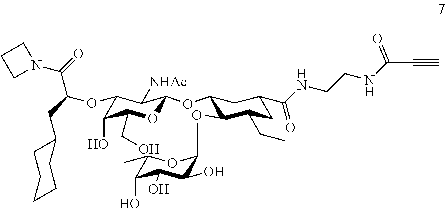

Compound 7: A solution of compound 6 (0.22 g, 0.28 mmole) and 3 drops of DIPEA in anhydrous DMF (3 mL) was cooled to 0° C. Propargylic acid NHS ester (57 mg, 0.34 mmole) was slowly added. The resulting solution was stirred for 1 hr. The solution was concentrated under reduced pressure and the residue was directly purified by Combi-flash [EtOAc/(MeOH/water, 6/1, v/v), 1/9-2/8, v/v). The product lyophilized to give compound 7 as an off-white solid (0.12 g, 0.15 mmole, 54%). MS: Calculated for C₄₀H₆₄N₄O₁₃=824.4, Found ES-positive m/z=847.3 (M+Na⁺).

Compound 9: To a slurry solution of compound 1 (0.12 g, 0.16 mmole) and DIPEA (0.1 mL) in anhydrous DMF (1 mL) was added a solution of azidoacetic acid-NHS ester (compound 8) (39 mg, 0.2 mmole) in anhydrous DMF (1 mL) dropwise over a 10 minute period at room temperature. The resulting solution was stirred for 3 hrs. The reaction mixture was concentrated under reduced pressure and the residue was purified by Combi-flash eluting with [EtOAc/(MeOH/water, 6/1, v/v), 9/1-2/8, v/v]. The product was collected then lyophilized to give compound 9 as a white solid (0.11 g, 0.13 mmole, 81%). MS: Calculated for C₃₇H₅₉N₃O₁₅=816.4, Found ES-positive m/z=838.7 (M+Na⁺), ES-negative m/z=814.7 (M−H).

Compound 11: A solution of PEG-17 Bis-NHS ester (compound 10) (0.2 g, 0.19 mmol) in DMSO (2 mL) was added to a solution of compound 1 (0.4 g, 0.56 mmole) and DIPEA (0.2 mL) in anhydrous DMSO (2 mL) dropwise over a 5 minute period at room temperature. The resulting solution was stirred overnight. The solution was dialyzed against distilled water for 3 days with dialysis tube MWCO 1000 while distilled water was changed every 12 hours. The solution in the tube was lyophilized overnight to give compound 11 as a white solid (0.32 g, 0.14 mmole, 77%).

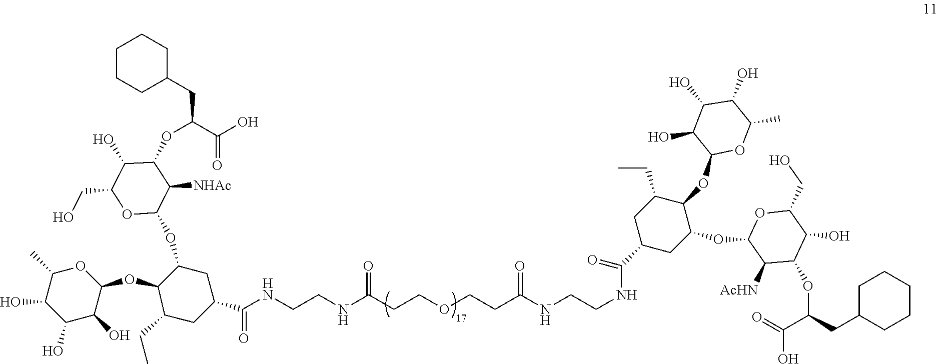

1H NMR (400 MHz, Deuterium Oxide) δ 5.02 (d, J=3.9 Hz, 2H), 4.90 (q, J=6.7 Hz, 2H), 4.52 (broad d, J=8.4 Hz, 2H), 3.97 (broad t, 2H), 3.86-3.74 (m, 16H), 3.73-3.59 (m, 62H), 3.56 (t, J=5.8 Hz 2H), 3.44 (m, 2H), 3.34-3.26 (m, 10H), 2.50 (t, J=6.1 Hz 4H), 2.31 (broad t, 2H), 2.12 (m, 2H), 2.04 (s, 6H), 1.90-1.79 (m, 4H), 1.78-1.38 (m, 14H), 1.37-1.26 (m, 14H), 1.25-1.08 (m, 14H), 0.98-0.79 (m, 10H). MS: Calculated for C₁₀₆H₁₈₈N₆O₄₇=2297.2, Found MALDI-TOF m/z=2321, (M+Na⁺).

### Example 2: Compound 12

Compound 12: Prepared in an analogous manner from compound 1 and PEG-25 bis-NHS ester.

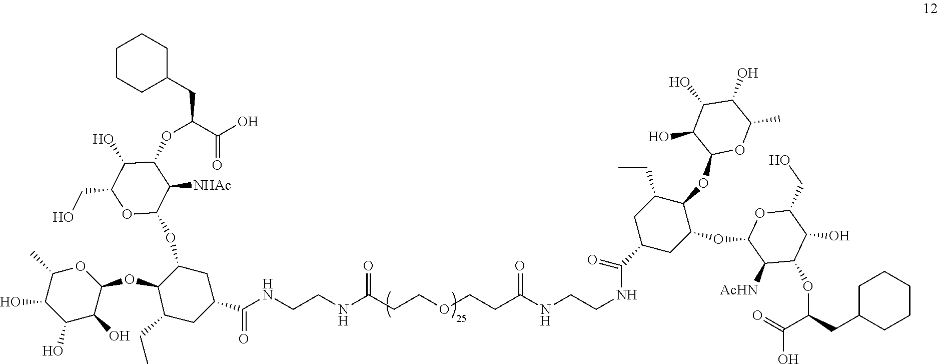

1H NMR (400 MHz, Deuterium Oxide) δ 5.03 (d, J=3.9 Hz, 2H), 4.91 (q, J=6.9 Hz, 2H), 4.53 (broad d, J=8.4 Hz, 2H), 3.98 (broad t, J=8.8 Hz 2H), 3.92-3.86 (m, 6H), 3.81-3.79 (m, 2H), 3.78-3.74 (m, 4H), 3.72-3.66 (m, 100H), 3.56 (t, J=5.7 Hz 2H), 3.52-3.40 (m, 2H), 3.37-3.25 (m, 10H), 2.53-2.49 (t, J=6.1 Hz 4H), 2.31 (m, 2H), 2.16-2.13 (m, 2H), 2.05 (s, 6H), 1.86-1.84 (m, 4H), 1.76-1.65 (m, 4H), 1.63-1.44 (m, 10H), 1.41-1.29 (m, 14H), 1.27-1.12 (m, 14H), 0.94-0.89 (m, 4H), 0.87-0.84 (t, J=7.2 Hz, 6H). MS: Calculated for C₁₂₂H₂₂₀N₆O₅₅=2649; Found MALDI-TOF m/z=2672 (M+Na⁺).

### Example 3: Compound 13

Compound 13: Prepared in an analogous manner from compound 1 and PEG-21 bis-NHS ester.

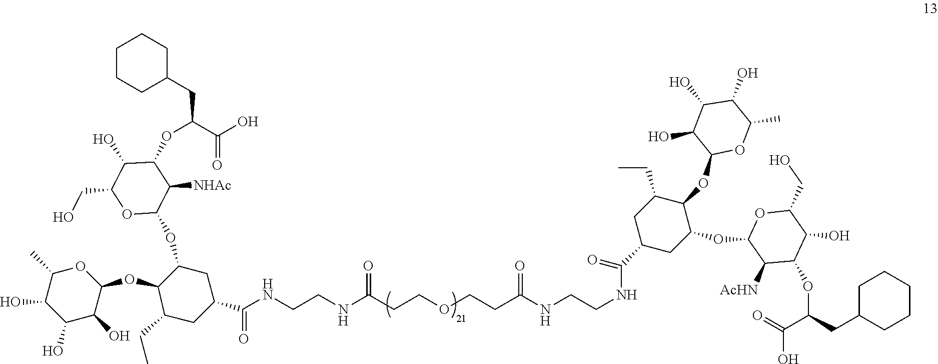

¹H NMR (400 MHz, Deuterium Oxide) δ 5.03 (d, J=3.9 Hz, 2H), 4.91 (q, J=6.7 Hz, 2H), 4.56 (broad d, J=8.4 Hz, 2H), 3.98 (broad t, 2H), 3.91-3.86 (m, 6H), 3.81-3.79 (m, 4H), 3.78-3.74 (m, 4H), 3.72 (m, 4H), 3.71-3.66 (m, 78H), 3.56 (t, J=5.8 Hz 2H), 3.47 (m, 2H), 3.35-3.27 (m, 10H), 2.53-2.49 (t, J=6.1 Hz 4H), 2.31 (broad t, 2H), 2.16-2.13 (m, 2H), 2.05 (s, 6H), 1.86-1.84 (m, 4H), 1.76-1.65 (m, 4H), 1.63-1.47 (m, 8H), 1.38-1.29 (m, 14H), 1.27-1.22 (m, 8H), 1.18-1.12 (m, 6H), 0.94-0.89 (m, 4H), 0.87-0.84 (t, J=7.2 Hz, 6H). MS: Calculated for C₁₁₄H₂₀₄N₆O₅₁=2473.3; Found MALDI-TOF m/z=2496 (M+Na⁺).

### Example 4: Compound 14

Compound 14: Prepared in an analogous manner from compound 1 and PEG-13 bis-NHS ester.

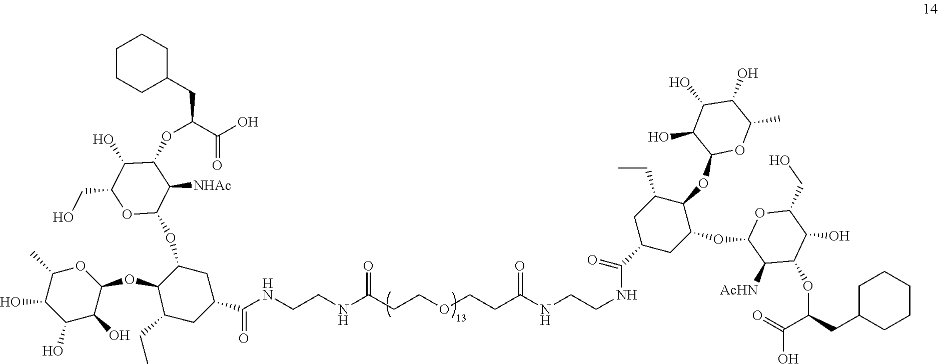

¹H NMR (400 MHz, Deuterium Oxide) δ 5.06 (d, J=4.1 Hz, 2H), 4.94 (q, J=6.6 Hz, 2H), 4.56 (broad d, J=8.4 Hz, 2H), 4.02 (Broad s, 2H), 3.94-3.90 (m, 6H), 3.84 (m, 2H), 3.80 (m, 4H), 3.76 (m, 6H), 3.72-3.70 (m, 50H), 3.59 (broad t, 2H), 3.49 (m, 2H), 3.38-3.33 (m, 10H), 2.54 (t, J=6.1 Hz 4H), 2.34 (broad t, 2H), 2.19-2.17 (m, 2H), 2.09 (s, 6H), 1.90-1.87 (m, 4H), 1.79-1.71 (m, 4H), 1.69-1.58 (m, 8H), 1.56 (m, 2H), 1.51 (m, 4H), 1.43-1.36 (m, 14H), 1.35-1.33 (m, 6H), 1.27-1.17 (m, 8H), 1.00-0.91 (m, 4H), 0.90-0.88 (t, 0.1=7.4 Hz, 6H). MS: Calculated for C₉₈H₁₇₂N₆O₄₃=2121.1; Found MALDI-TOF m/z=2144 (M+Na⁺).

### Example 5: Compound 15

Compound 15: Prepared in an analogous manner from compound 1 and PEG-10 bis-NHS ester.

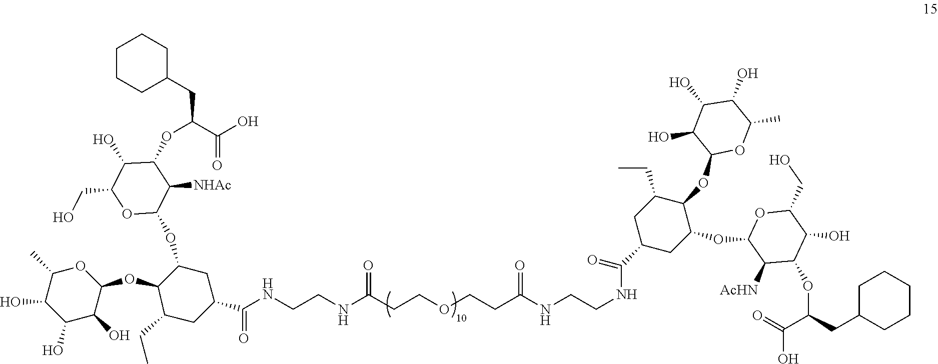

¹H NMR (400 MHz, Deuterium Oxide) δ 5.06 (d, J=4.0 Hz, 2H), 4.94 (q, J=6.7 Hz, 2H), 4.56 (broad d, J=8.4 Hz, 2H), 4.02 (broad s, 2H), 3.95-3.90 (m, 6H), 3.84 (m, 2H), 3.79 (m, 4H), 3.75 (m, 6H), 3.72 (m, 30H), 3.70 (broad s, 10H), 3.58 (broad t, J=5.6 Hz 2H), 3.51 (m, 2H), 3.38-3.35 (m, 6H), 3.34-3.31 (m, 4H), 2.54 (t, 4H), 2.34 (broad t, 2H), 2.19-2.17 (m, 2H), 2.09 (s, 6H), 1.90-1.87 (m, 4H), 1.79-1.66 (m, 4H), 1.63-1.55 (m, 8H), 1.53-1.49 (m, 2H), 1.41 (q, J=12.0 Hz, 4H), 1.37-1.32 (m, 8H), 1.27 (broad d, J=6.6 Hz, 6H), 1.24-1.17 (m, 8H), 0.98-0.93 (m, 4H), 0.90-0.88 (t, J=7.4 Hz, 6H). MS: Calculated for C₉₂H₁₈₀N₆O₄₀=1989.0; Found MALDI-TOF m/z=2013 (M+N).

### Example 6: Compound 16

Compound 16: Prepared in an analogous manner from compound 1 and PEG-9 bis-NHS ester.

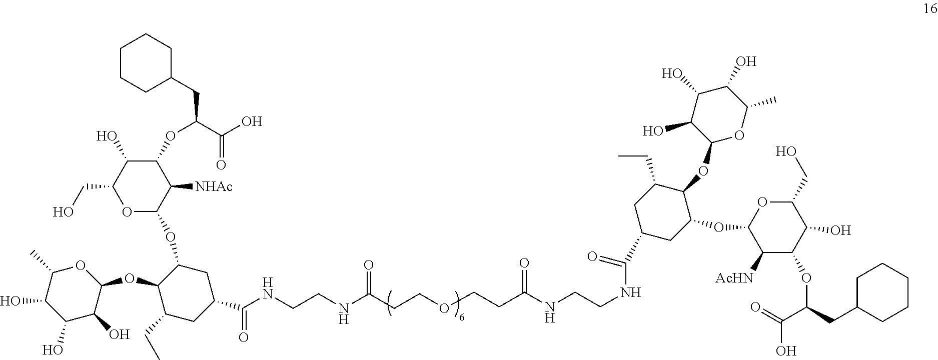

¹H NMR (400 MHz, Deuterium Oxide) δ 7.82 (m, 2H), 6.83 (d, J=8.9 Hz, 2H), 4.91 (d, J=4.0 Hz, 2H), 4.79 (q, J=6.7 Hz, 2H), 4.39 (d, J=8.5 Hz, 2H), 3.96-3.83 (m, 4H), 3.81 (d, J=3.0 Hz, 2H), 3.79-3.71 (m, 4H), 3.71-3.47 (m, 34H), 3.47-3.31 (m, 4H), 3.31-3.07 (m, 10H), 2.39 (t, J=6.1 Hz, 4H), 2.19 (t, J=12.5 Hz, 2H), 2.03 (broad d, J=6.8 Hz, 2H), 1.93 (s, 6H), 1.73 (broad d, J=12.5 Hz, 4H), 1.68-1.34 (m, 16H), 1.34-1.15 (m, 4H), 1.15-0.91 (m, 14H), 0.91-0.65 (m, 10H). MS: Calculated for C₈₄H₁₄₄N₆O₃₆=1812.9; Found ES-Negative M/Z=1812.8 (M−1).

### Example 7: Compound 17

Compound 17: Prepared in an analogous manner from compound 1 and PEG-4 bis-NHS ester.

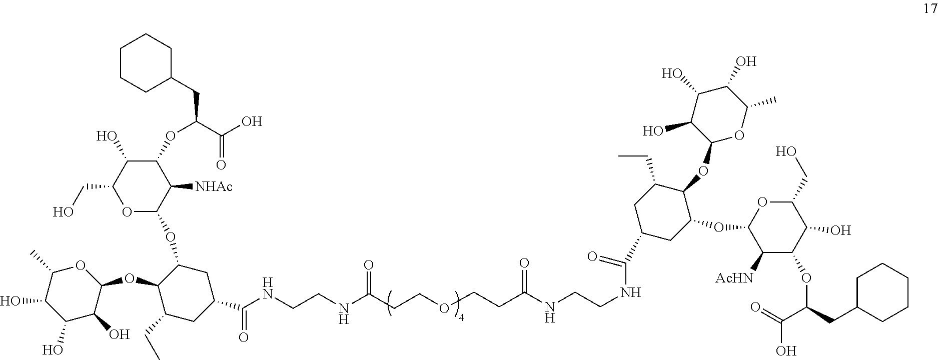

¹H NMR (400 MHz, Deuterium Oxide) δ 4.91 (d, J=4.0 Hz, 2H), 4.80 (q, J=6.7 Hz, 2H), 4.40 (broad d, J=8.4 Hz, 2H), 4.00-3.84 (m, 4H), 3.82 (d, J=3.0 Hz, 2H), 3.76 (dd, J=10.6, 3.2 Hz, 2H), 3.72-3.57 (m, 12H), 3.55 (m, J=3.1 Hz, 14H), 3.42 (m, J=7.5, 4.5 Hz, 4H), 3.30-3.09 (m, 10H), 2.39 (t, J=6.1 Hz, 4H), 2.20 (broad t, J=12.6 Hz, 2H), 2.03 (m, J=6.5 Hz, 2H), 1.94 (s, 6H), 1.73 (broad d, J=12.5 Hz, 4H), 1.67-1.33 (m, 16H), 1.33-0.93 (m, 20H), 0.89-0.67 (m, 10H). MS: Calculated for C₈₀H₁₃₆N₆O₃₄=1724.9; Found ES-Negative m/z=1724.8 (M−1).

### Example 8: Compound 18

Compound 18: Prepared in an analogous manner from compound 1 and PEG-2 bis-NHS ester.

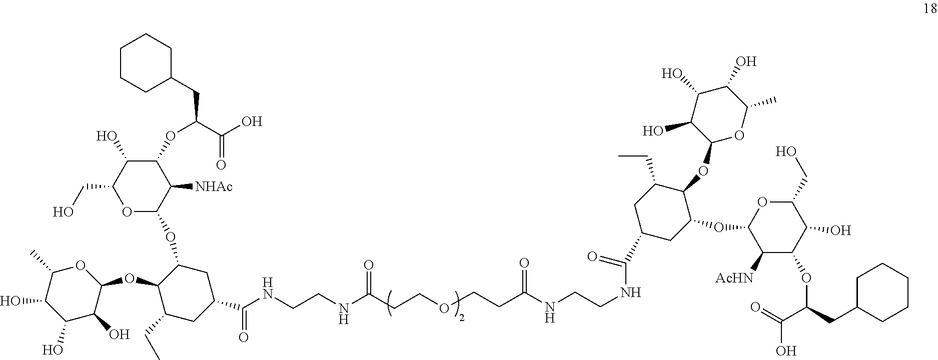

¹H NMR (400 MHz, Deuterium Oxide) δ 4.91 (d, J=4.0 Hz, 2H), 4.79 (q, J=6.7 Hz, 2H), 4.40 (broad d, J=8.5 Hz, 2H), 4.01-3.84 (m, 4H), 3.81 (d, J=3.0 Hz, 2H), 3.76 (dd, J=10.5, 3.2 Hz, 2H), 3.72-3.55 (m, 14H), 3.52 (s, 4H), 3.42 (m, J=6.0 Hz, 4H), 3.28-3.06 (m, 10H), 2.38 (t, J=6.1 Hz 4H), 2.19 (broad t, J=12.7 Hz, 2H), 2.03 (m, J=6.5 Hz, 2H), 1.94 (s, 6H), 1.73 (m, J=12.5 Hz, 4H), 1.67-1.33 (m, 16H), 1.33-0.92 (m, 20H), 0.92-0.60 (m, 10H). MS: Calculated for C₇₆H₁₂₈N₆O₃₂=1636.8; Found ES-Negative m/z=1636.7 (M−1).

### Example 9: Compound 19

Compound 19: Prepared in an analogous manner from compound 1 and succinic acid bis-NHS ester.

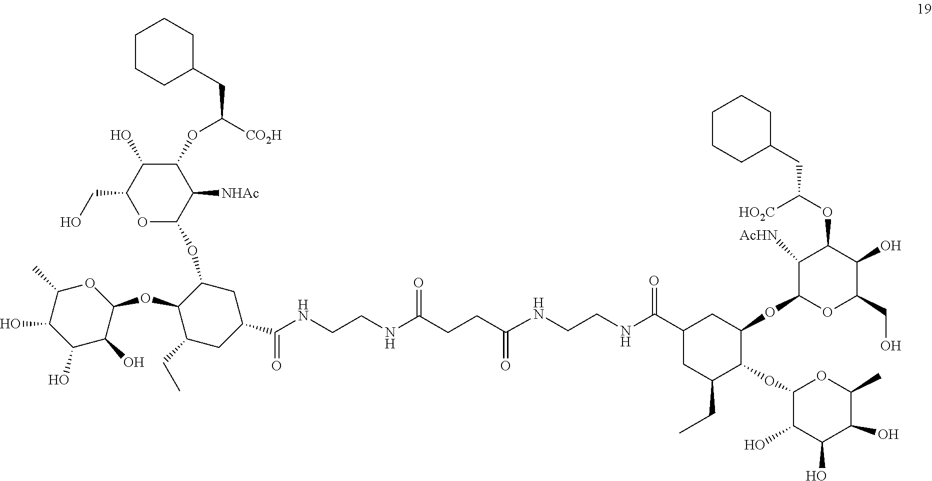 [SMILES: CC[C@H]1CC(C(=O)NCCNC(=O)CCC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)O)[C@H]3NC(C)=O)C2)C[C@@H](O[C@@H]2O[C@H](CO)[C@H](O)[C@H](O[C@@H](CC3CCCCC3)C(=O)O)[C@H]2NC(C)=O)[C@@H]1O[C@@H]1O[C@@H](C)[C@@H](O)[C@@H](O)[C@@H]1O]

¹H NMR (400 MHz, Deuterium Oxide) δ 4.91 (d, J=4.0 Hz, 2H), 4.80 (q, J=6.8 Hz, 2H), 4.41 (broad d, J=8.6 Hz, 2H), 3.88 (m, 2H), 3.81-3.74 (m, 6H), 3.73-3.65 (m, 6H), 3.64-3.56 (m, 6H), 3.45 (broad t, 2H), 3.33 (broad d, J=9.9 Hz, 2H), 3.20 (m, J=11.4, 10.3 Hz, 10H), 2.39 (s, 4H), 2.19 (m, J=12.8 Hz, 2H), 2.02 (m, 2H), 1.94 (s, 6H), 1.84-1.69 (m, 4H), 1.51 (m, J=65.3, 30.1, 14.0 Hz, 14H), 1.26 (q, J=12.5 Hz, 6H), 1.09 (m, J=28.4, 8.7 Hz, 14H), 0.94-0.64 (in, 10H). MS: Calculated for C₇₂H₁₂₀N₆O₃₀=1548.8; Found ES-Negative m/z=1548.67 (M−1).

### Example 10: Compound 20

Compound 20: A solution of compound 15 (12.4 mg, 6.23 μmole) and DIPEA (11 μL, 62.3 μmole) in anhydrous DMF (0.2 mL) was cooled to 0° C. and TBTU (12 mg, 37.8 μmole) was added. The resulting solution was stirred for 10 minute. Azetidine (8.4 μL, 124.6 μmole) was added and the resulting solution was stirred for 1 h at room temperature. The reaction mixture was concentrated under high vacuum and the residue was purified by HPLC. The product portion was collected and evaporated, re-dissolved in minimum amount of distilled water then lyophilized overnight to give compound 20 as a white solid (6.3 mg, 49%).

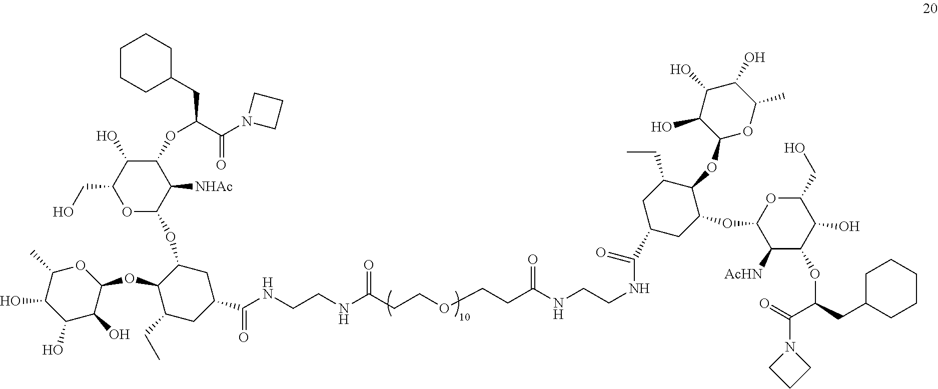

¹H NMR (400 MHz, Deuterium Oxide) δ 8.32 (s, 2H), 8.23 (d, J=9.5 Hz, 2H), 4.92 (broad d, 2H), 4.79 (q, J=6.7 Hz, 2H), 4.42 (m, 2H), 4.23 (q, J=7.8 Hz, 2H), 4.14 (q, J=7.8 Hz, 2H), 4.06-3.79 (m, 6H), 3.76 (dd, J=10.5 Hz, 2H), 3.66 (m, J=15.1, 13.8, 8.6 Hz, 8H), 3.57 (m, J=8.0 Hz, 46H), 3.41 (m, 4H), 3.21 (m, J=14.4, 12.2 Hz, 10H), 2.45-2.34 (t, 4H), 2.22 (m, J=12.9 Hz, 6H), 2.02 (m, 2H), 1.94 (s, 6H), 1.74 (broad d, J=12.2 Hz, 4H), 1.68-1.33 (m, 14H), 1.26 (m, J=11.1 Hz, 6H), 1.15-0.95 (m, 16H), 0.95-0.64 (m, 10H). MS: Calculated for C₉₈H₁₇₀N₈O₃₈=2067; Found ES-Negative m/z=1033.6 ((M−1)/2).

The following compounds were prepared in an analogous manner:

### Example 11: Compound 21

Compound 21: Prepared in an analogous manner from compound 15 and dimethylamine.

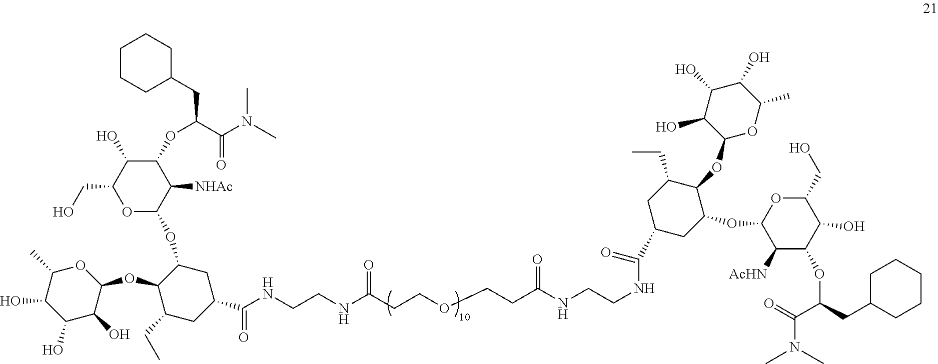

¹H NMR (400 MHz, Deuterium Oxide) δ 8.33 (s, 6H), 4.93 (broad s, 2H), 4.80 (q, 2H), 4.42 (broad d, J=9.9 Hz, 4H), 3.89 (broad s, 2H), 3.77 (dd, J=10.9 Hz, 2H), 3.74-3.49 (m, 54H), 3.42 (Broad s, 4H), 3.21 (m, J=14.5, 12.4 Hz, 10H), 2.95 (s, 6H), 2.83 (s, 6H), 2.41 (broad t, 4H), 2.21 (broad t, 2H), 2.05 (m, 2H), 1.97 (s, 6H), 1.73 (m, 6H), 1.67-1.36 (m, 12H), 1.36-0.96 (m, 20H), 0.80 (d, J=38.2 Hz, 10H). MS: Calculated for C₉₆H₁₇₀N₈O₃₈=2043.0; Found ES-Negative m/z=1066.8 ((M+formic acid−1)/2).

### Example 12: Compound 22

Compound 22: Prepared in an analogous manner from compound 12 and azetidine.

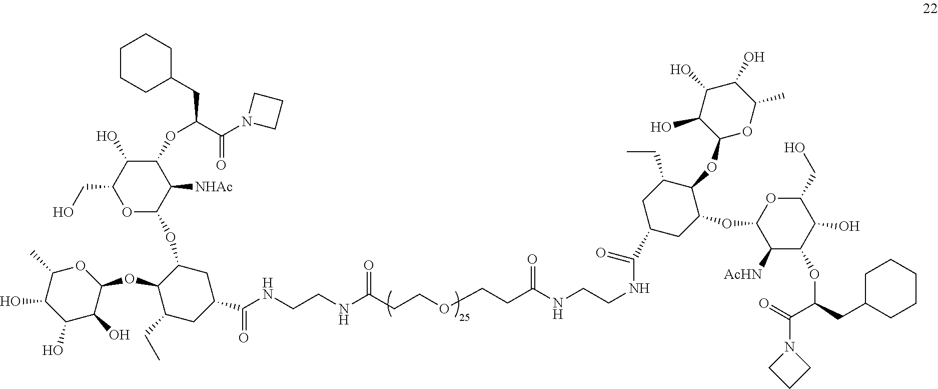

¹H NMR (400 MHz, Deuterium Oxide) δ 8.33 (s, 2H), 4.92 (d, J=4.0 Hz, 2H), 4.79 (q, J=6.6 Hz, 2H), 4.42 (Broad d, J=8.6 Hz, 2H), 4.24 (q, J=8.7 Hz, 2H), 4.15 (q, J=8.6 Hz, 2H), 3.96 (m, J=25.2, 9.1 Hz, 4H), 3.86 (broad s, 2H), 3.77 (dd, J=10.6, 3.1 Hz, 2H), 3.73-3.47 (m, 114H), 3.42 (m, J=7.8, 4.6 Hz, 4H), 3.20 (m, J=22.8, 8.6 Hz, 10H), 2.41 (t, J=6.1 Hz, 4H), 2.35-2.13 (m, 6H), 2.04 (m, J=10.8 Hz, 2H), 1.95 (s, 6H), 1.75 (broad d, J=12.7 Hz, 4H), 1.68-1.35 (m, 16H), 1.35-0.94 (m, 20H), 0.94-0.67 (m, 10H). MS: Calculated for C₁₂₈H₂₃₀N₈O₅₃=2727.5; Found ES-Negative m/z=1409.3 ((M+formic acid−1)/2).

### Example 13: Compound 23

Compound 23: Prepared in an analogous manner from compound 17 and azetidine.

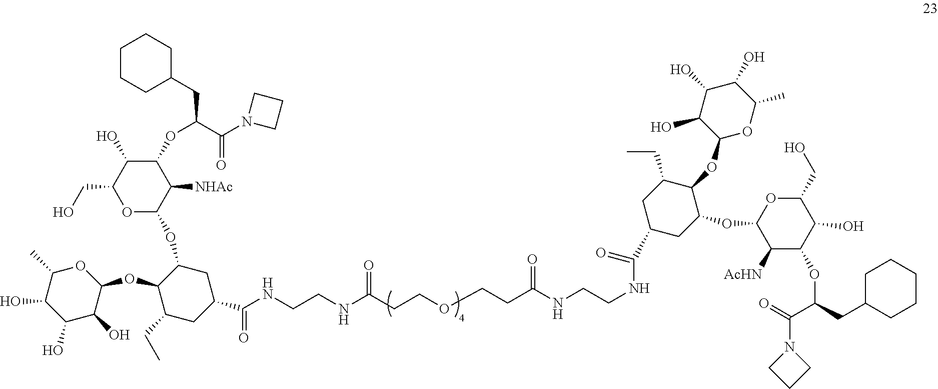

¹H NMR (400 MHz, Deuterium Oxide) δ 8.28 (broad s, 2H), 8.23 (broad d, 2H), 4.91 (d, J=4.0 Hz, 2H), 4.78 (q, J=7.4, 6.9 Hz, 2H), 4.41 (broad d, J=8.5 Hz, 2H), 4.23 (q, J=8.7 Hz, 2H), 4.14 (q, J=8.8 Hz, 2H), 4.04-3.80 (m, 8H), 3.76 (dd, J=10.6, 3.2 Hz, 2H), 3.72-3.58 (m, 16H), 3.55 (d, J=3.0 Hz, 12H), 3.41 (m, J=7.7, 4.4 Hz, 4H), 3.30-3.10 (m, 10H), 2.40 (t, J=6.1 Hz, 4H), 2.34-2.12 (m, 6H), 2.03 (m, J=7.1 Hz, 2H), 1.94 (s, 6H), 1.74 (broad d, J=12.7 Hz, 4H), 1.67-1.33 (m, 14H), 1.33-1.16 (m, 8H), 1.16-0.95 (m, 14H), 0.95-0.64 (m, 10H). MS: Calculated for C₈₆H₁₄₆N₈O₃₂=1803.0; Found ES-Positive m/z=1826.8 (M+Na⁺).

### Example 14: Compound 24

Compound 24: Prepared in an analogous manner from compound 16 and azetidine.

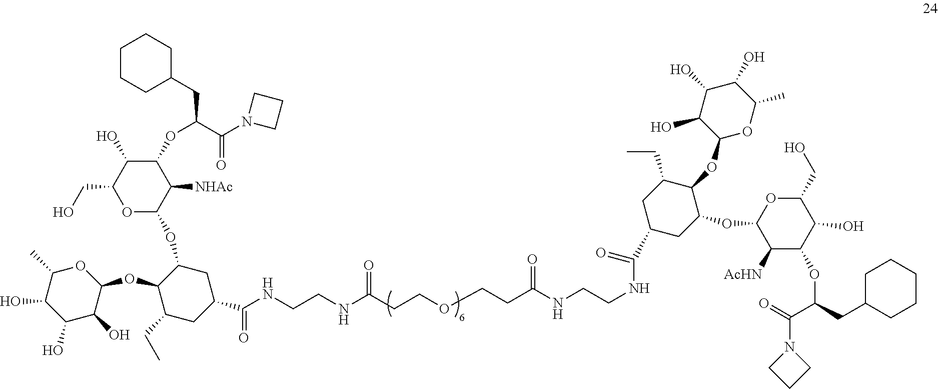

¹H NMR (400 MHz, Deuterium Oxide) δ 4.92 (d, J=4.0 Hz, 2H), 4.79 (q, J=6.6 Hz, 2H), 4.42 (m, 2H), 4.24 (q, J=8.7 Hz, 2H), 4.14 (q, J=8.4 Hz, 2H), 3.96 (m, J=24.9, 8.9 Hz, 8H), 3.80-3.48 (m, 36H), 3.42 (m, J=7.7, 4.4 Hz, 4H), 3.19 (m, J=23.4, 8.5 Hz, 10H), 2.40 (t, J=6.1 Hz, 4H), 2.32-2.10 (m, 8H), 2.02 (m, 2H), 1.94 (s, 6H), 1.74 (broad d, J=12.5 Hz, 4H), 1.67-1.34 (m, 14H), 1.24 (m, J=11.2 Hz, 8H), 1.16-0.94 (m, 14H), 0.94-0.64 (m, 10H). MS: Calculated for C₉₀H₁₅₄N₈O₃₄=1891.0; Found ES-Negative m/z=1935.9 (M+formic acid−1).

### Example 15: Compound 25

Compound 25: Prepared in an analogous manner from compound 18 and azetidine.

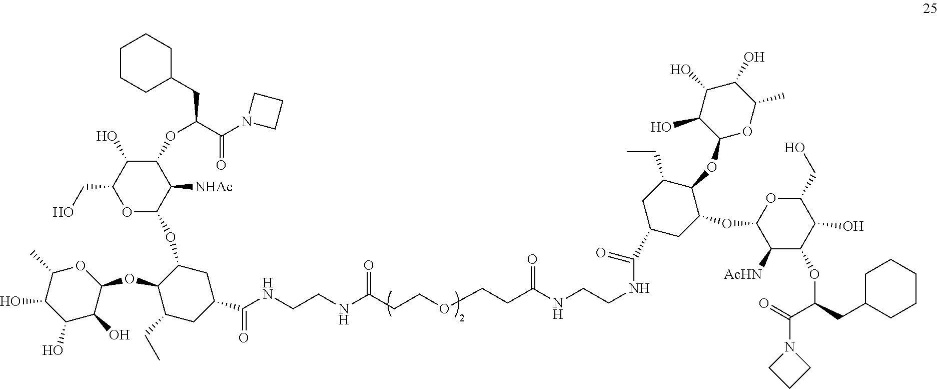

¹H NMR (400 MHz, Deuterium Oxide) δ 8.23 (d, J=9.6 Hz, 2H), 4.91 (d, J=4.0 Hz, 2H), 4.78 (q, J=6.7 Hz, 2H), 4.41 (broad d, J=8.5 Hz, 2H), 4.23 (q, J=8.6 Hz, 2H), 4.14 (q, J=8.7 Hz, 2H), 3.95 (m, =24.6, 8.8 Hz, 8H), 3.76 (dd, J=10.6, 3.2 Hz, 2H), 3.72-3.55 (m, 14H), 3.53 (s, 4H), 3.41 (m, J=7.7, 4.4 Hz, 4H), 3.19 (m, J=13.5, 10.9 Hz, 12H), 2.39 (t, J=6.1 Hz, 4H), 2.21 (m, J=16.1, 8.8 Hz, 6H), 2.02 (m, 2H), 1.94 (s, 6H), 1.74 (broad d, J=12.4 Hz, 4H), 1.67-1.33 (m, 14H), 1.33-0.93 (m, 22H), 0.93-0.62 (m, 10H). MS: Calculated for C₈₂H₁₃₈N₈O₃₀=1714.9; Found ES-Positive m/z=1737.8 (M+Na⁺).

### Example 16: Compound 27

Compound 27: To a mixture of compound 2 (72 mg, 91 μmole) and compound 26 (azido-PEG3-azide) (9.3 mg, 38 μmole) in deionized water (2 mL) was added a solution of CuSO₄-THPTA (0.04M) (0.5 mL) and sodium ascorbate (38 mg, 0.19 mmole) successively. The reaction mixture was stirred overnight at room temperature. The reaction mixture was concentrated under high vacuum and the residue was purified by HPLC. The product was lyophilized overnight to give compound 27 as a white solid (3.0 mg, 4%).

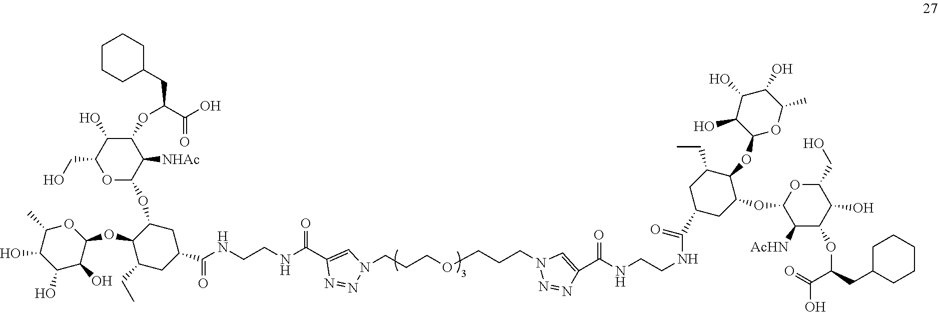

1H NMR (400 MHz, Deuterium Oxide) δ 8.27 (s, 2H), 8.22 (s, 2H), 4.88 (d, J=PGP-1, 4.0 Hz, 2H), 4.78 (q, J=6.8 Hz, 2H), 4.53 (t, J=4.9 Hz, 4H), 4.39 (broad d, J=8.6 Hz, 2H), 3.94-3.80 (m, 8H), 3.80-3.72 (m, 4H), 3.72-3.64 (m, 4H), 3.60 (m, J=5.8 Hz, 4H), 3.54-3.31 (m, 18H), 3.31-3.09 (m, 4H), 2.16 (broad t, J=12.6 Hz, 2H), 2.01 (m, J=7.5 Hz, 2H), 1.90 (s, 6H), 1.80-1.30 (m, 20H), 1.22 (m, J=11.9 Hz, 2H), 1.16-0.87 (m, 18H), 0.78 (m, J=23.1, 10.9 Hz, 4H), 0.63 (t, J=7.3 Hz, 6H). MS: Calculated for C82H134N12O33=1814.9; Found ES-Negative m/z=1814.7 (M−1).

The following compounds were prepared in an analogous manner:

### Example 17: Compound 28

Compound 28: Prepared in an analogous manner from compound 2 and azido-PEG2-azide.

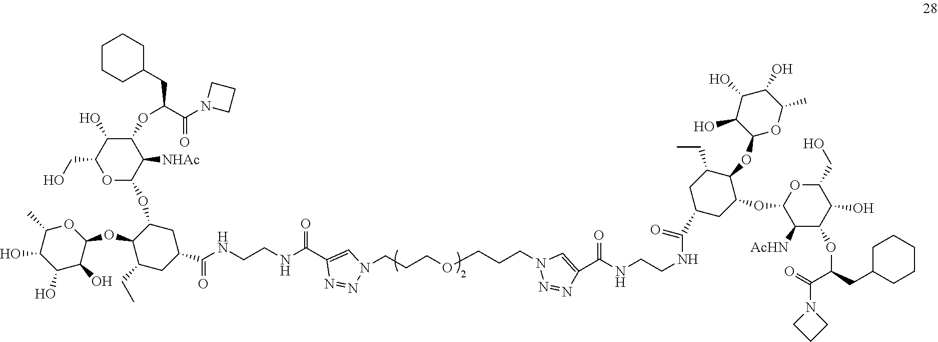

¹H NMR (400 MHz, Deuterium Oxide) δ 8.23 (s, 2H), 4.87 (d, J=4.0 Hz, 2H), 4.77 (q, J=6.9 Hz, 2H), 4.50 (t, J=4.9 Hz, 4H), 4.37 (broad d, J=8.6 Hz, 2H), 3.87 (broad d, J=5.9 Hz, 4H), 3.82-3.71 (m, 8H), 3.71-3.63 (m, 4H), 3.63-3.53 (m, 4H), 3.50 (m, 6H), 3.46-3.32 (m, 8H), 3.32-3.23 (m, 2H), 3.23-3.09 (m, 2H), 2.17 (broad t, J=12.8 Hz, 2H), 2.10-1.97 (m, 2H), 1.89 (s, 6H), 1.82-1.30 (m, 20H), 1.21 (d, J=12.1 Hz, 4H), 1.16-0.87 (m, 18H), 0.79 (dt, J=22.3, 10.7 Hz, 4H), 0.62 (t, J=7.4 Hz, 6H). MS: Calculated for C₈₀H₁₃₀N₁₂O₃₂=1770.8; Found ES-Negative m/z=1769.7 (M−1).

### Example 18: Compound 29

Compound 29: To a solution of compound 7 (46 mg, 56 μmole) and compound 26 (azido-PEG3-azide) (5.6 mg, 23 μmole) in a solution of MeOH (3 mL) and distilled water (0.3 mL) was added a solution of CuSO₄-THPTA (0.04M) (0.3 mL) and sodium ascorbate (23 mg, 0.12 mmole) successively. The resulting solution was stirred overnight at room temperature. To complete the reaction, another set of catalyst was added and the reaction was continued additional 6 hrs. After the reaction was completed, the solution was concentrated under high vacuum and the residue was purified by HPLC. The product portion was collected and evaporated, re-dissolved in minimum amount of distilled water then lyophilized overnight to give compound 29 as a white solid (25.2 mg, 13.3 μmole, 57%).

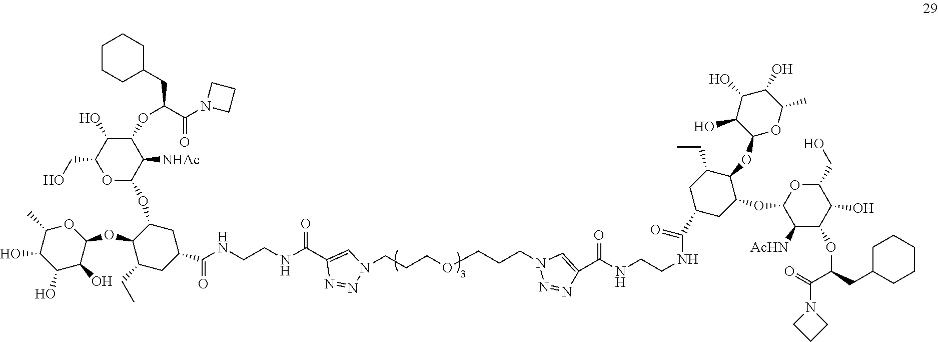

¹H NMR (400 MHz, Deuterium Oxide) δ 8.28 (s, 2H), 4.88 (d, J=4.0 Hz, 2H), 4.77 (q, J=6.8 Hz, 2H), 4.53 (t, J=4.8 Hz, 4H), 4.38 (broad d, 2H), 4.23 (q, J=7.7 Hz, 2H), 4.13 (q, J=8.4 Hz, 2H), 4.07-3.87 (m, 6H), 3.82 (t, J=4.9 Hz, 4H), 3.79-3.63 (m, 8H), 3.63-3.55 (m, 6H), 3.55-3.32 (m, 14H), 3.32-3.10 (m, 4H), 2.33-2.08 (m, 8H), 2.02 (m, 2H), 1.89 (s, 6H), 1.81-1.31 (m, 18H), 1.22 (m, J=11.6 Hz, 6H), 1.17-0.90 (m, 14H), 0.90-0.68 (m, 4H), 0.63 (t, J=7.3 Hz, 6H). MS: Calculated for C₈₈H₁₄₄N₁₄O₃₁=1893.0; Found ES-Positive m/z=969.5 (M/2+Na⁺).

The following compounds were prepared in an analogous manner:

### Example 19: Compound 30

Compound 30: Prepared in an analogous manner from compound 7 and azido-PEG5-azide.

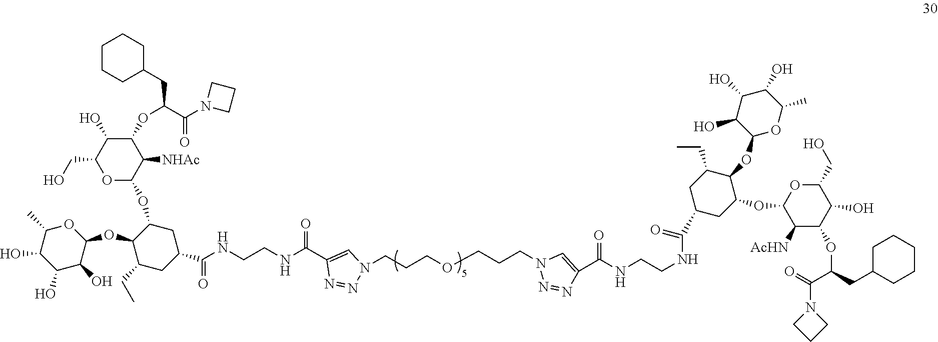

¹H NMR (400 MHz, Deuterium Oxide) δ 8.33 (s, 2H), 4.88 (d, J=3.9 Hz, 2H), 4.77 (q, J=6.8 Hz, 2H), 4.55 (t, J=5.0 Hz, 4H), 4.39 (m, 2H), 4.22 (q, J=8.2 Hz, 2H), 4.13 (q, J=8.7 Hz, 2H), 4.00 (broad d, J=9.9 Hz, 2H), 3.93 (q, J=7.7 Hz, 4H), 3.85 (t, J=5.0 Hz, 4H), 3.74 (dd, 1=10.5, 3.2 Hz, 2H), 3.70 (broad d, J=3.0 Hz, 2H), 3.69-3.62 (m, 4H), 3.59 (m, J=7.7 Hz, 6H), 3.53 (m, J=5.6 Hz, 2H), 3.47 (m, J=11.4, 4.1 Hz, 12H), 3.43-3.31 (m, 6H), 3.31-3.22 (m, 2H), 3.17 (t, J=9.7 Hz, 2H), 2.20 (m, J=14.0 Hz, 8H), 2.01 (m, J=10.3 Hz, 2H), 1.90 (s, 6H), 1.75-1.31 (m, 18H), 1.22 (m, J=12.1 Hz, 6H), 1.16-0.91 (m, 14H), 0.91-0.69 (m, 4H), 0.63 (t, J=7.3 Hz, 6H). MS: Calculated for C₉₂H₁₅₂N₁₄O₃₃=1981.0; Found ES-Positive m/z=1013.6 (M/2+Na⁺).

### Example 20: Compound 31

Compound 31: To a solution of compound 2 (30 mg, 38 μmole) and compound 9 (46 mg, 57 μmole) in distilled water (2 mL) was added a solution of CuSO₄-THPTA (0.04M) (0.2 mL) and sodium ascorbate (1.5 mg, 7.6 μmole) successively. The resulting solution was stirred for 4 hrs at room temperature. The solution was concentrated under high vacuum and the residue was purified by HPLC. The product portion was collected and evaporated, re-dissolved in minimum amount of distilled water then lyophilized overnight to give compound 31 as a white solid (3.5 mg, 6%).

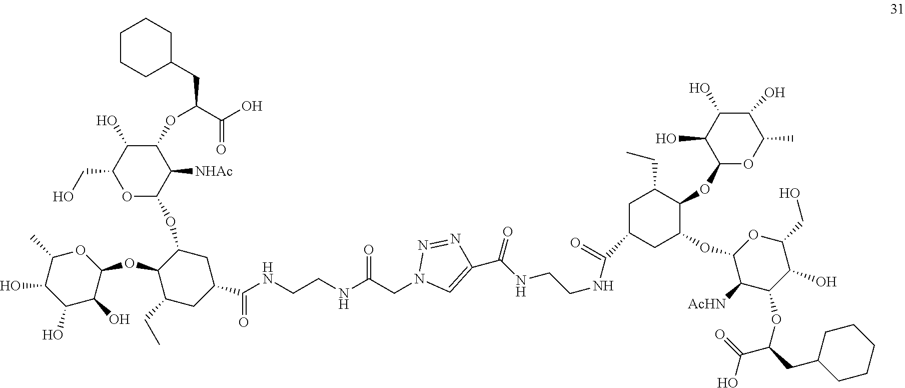 [SMILES: CC[C@H]1C[C@@H](C(=O)NCCNC(=O)Cn2cc(C(=O)NCCNC(=O)[C@@H]3C[C@H](CC)[C@@H](O[C@@H]4O[C@@H](C)[C@@H](O)[C@@H](O)[C@@H]4O)[C@H](O[C@@H]4O[C@H](CO)[C@H](O)[C@H](O[C@@H](CC5CCCCC5)C(=O)O)[C@H]4NC(C)=O)C3)nn2)C[C@@H](O[C@@H]2O[C@H](CO)[C@H](O)[C@H](O[C@@H](CC3CCCCC3)C(=O)O)[C@H]2NC(C)=O)[C@@H]1O[C@@H]1O[C@@H](C)[C@@H](O)[C@@H](O)[C@@H]1O]

¹H NMR (400 MHz, Deuterium Oxide) δ 8.39 (s, 1H), 5.23 (s, 2H), 4.97 (t, J=4.5 Hz, 2H), 4.85 (m, 2H), 4.45 (broad t, 2H), 3.94 (m, 2H), 3.91-3.78 (m, 6H), 3.77-3.62 (m, 12H), 3.61-3.40 (m, 8H), 3.40-3.16 (m, 8H), 2.24 (m, J=12.0 Hz, 2H), 2.09 (m, 2H), 1.98 (two s, 6H), 1.89-1.37 (m, 20H), 1.36-1.24 (m, 4H), 1.23-0.94 (m, 18H), 0.93-0.77 (m, 4H), 0.71 (t, J=7.2 Hz, 6H). MS: Calculated for C₇₃H₁₁₉N₉O₃₀=1601.8; Found ES-Negative m/z=1600.5 (M−H).

### Example 21: Compound 32

Compound 32: To a solution of compound 1 (25 mg, 34 μmole) and carbonyldiimidazole (2.3 mg, 14 μmole) in anhydrous DMF (1 mL) was added DIPEA (20 μL). The resulting solution was stirred overnight at room temperature under an N₂ atmosphere. The reaction mixture was concentrated under high vacuum and the residue was purified by HPLC. The product portion was collected and evaporated, re-dissolved in minimum amount of distilled water then lyophilized overnight to give compound 32 as a white solid (1.6 mg, 8%).

Compound 32 (Alternative Synthesis): To a solution of compound 1 (0.77 g, 1.04 mmole) in anhydrous DMSO (3 mL) was added bis(p-nitrophenyl) carbonate (0.15 g, 0.49 mole) (3 mL). The reaction mixture was stirred overnight at 40° C. The reaction mixture was lyophilized to dryness. The residue was purified by reverse phase C-18 column chromatography eluting with a solution of water/MeOH (gradient change from 9/1 to 1/9 v/v). The product portion was concentrated and lyophilized to give the desired product as a white solid (0.47 g, 0.31 mmole, 48%).

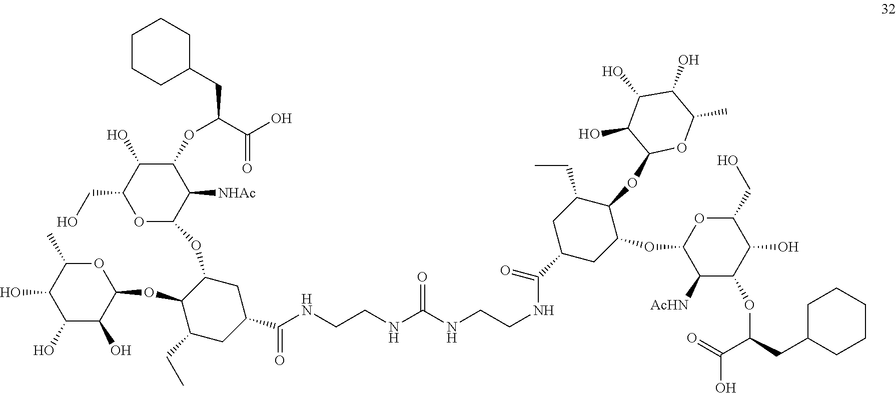 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)O)[C@H]3NC(C)=O)C2)C[C@@H](O[C@@H]2O[C@H](CO)[C@H](O)[C@H](O[C@@H](CC3CCCCC3)C(=O)O)[C@H]2NC(C)=O)[C@@H]1O[C@@H]1O[C@@H](C)[C@@H](O)[C@@H](O)[C@@H]1O]

¹H NMR (400 MHz, Deuterium Oxide) δ 4.92 (d, J=4.0 Hz, 2H), 4.81 (q, J=6.7 Hz, 2H), 4.42 (broad d, J=8.5 Hz, 2H), 3.88 (m, 2H), 3.84-3.74 (m, 6H), 3.73-3.56 (m, 12H), 3.45 (t, J=5.9 Hz, 2H), 3.36 (broad d, J=10.1 Hz, 2H), 3.29-3.00 (m, 12H), 2.23 (broad t, J=12.7 Hz, 2H), 2.05 (m, 2H), 1.95 (s, 6H), 1.75 (broad d, J=12.5 Hz, 411), 1.69-1.35 (m, 18H), 1.35-1.16 (m, 6H), 1.15-0.92 (m, 16H), 0.91-0.62 (in, 12H); MS: Calculated for C₆₉H₁₁₆N₆O₂₉=1492.7; Found ES-Negative m/z=1491.5 (M−H).

### Example 22: Intermediate 35

Compound 35: A solution of L-Lysine (OBn ester) (0.15 g, 0.49 mmole) in anhydrous DMF (3 mL) was cooled to 0° C. and DIPEA (0.35 mL, 2.0 mmole) was added. The solution was stirred for 10 min. This solution was added to a solution of N₃-PEG1-NHS ester (compound 34) (0.30 g, 1.16 mmole) over a 5 minute period followed by a catalytic amount of DMAP (20 mg). The resulting solution was stirred overnight while temperature was gradually increased to room temperature. The solution was concentrated and the residue was dried under high vacuum for 30 min to dryness, then directly purified by Combi-flash (EtOAc/MeOH, EtOAc only—2/1, v/v). The product portion was collected and evaporated, then dried under high vacuum to give compound 35 as a light yellow gel (0.25 g, 0.48 mmole, 98%). MS: Calculated (C23H34N8O6, 518.2), ES-positive (519.2, M+1, 541.2 M+Na).

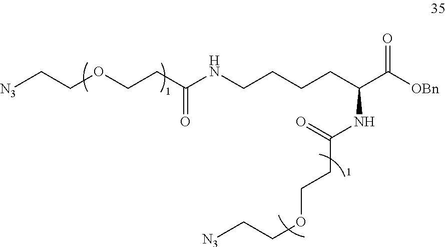

¹H NMR (400 MHz, Methanol-d₄) δ 7.47-7.22 (m, 5H), 5.31-5.03 (dd, 2H), 4.45 (dd, J=8.7, 5.2 Hz, 1H), 3.86-3.66 (m, 4H), 3.63 (q, J=4.9 Hz, 4H), 3.45-3.24 (m, 7H), 3.17 (td, J=6.9, 4.9 Hz, 2H), 2.63-2.48 (m, 2H), 2.45 (t, J=6.1 Hz, 2H), 1.86 (dtd, J=13.3, 8.0, 5.2 Hz, 1H), 1.80-1.63 (m, 1H), 1.63-1.45 (m, 2H), 1.39 (m, 2H).

### Example 23: Intermediate 36

Compound 36: Prepared in an analogous manner from compound 33 and azido-PEG5-NHS ester in 58% yield.

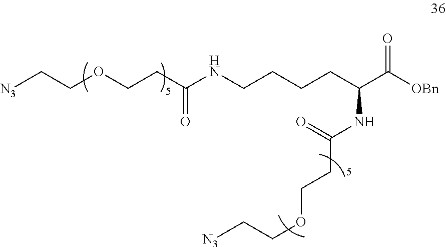

¹H NMR (400 MHz, Methanol-d₄) δ 7.48-7.26 (m, 5H), 5.29-5.09 (dd, 2H), 4.45 (dd, J=8.8, 5.2 Hz, 1H), 3.81-3.55 (m, 41H), 3.43-3.36 (m, 5H), 3.33 (p, J=1.7 Hz, 12H), 3.17 (t, J=7.0 Hz, 2H), 2.61-2.49 (m, 2H), 2.44 (t, J=6.1 Hz, 2H), 1.95-1.80 (m, 1H), 1.80-1.66 (m, 1H), 1.61-1.46 (m, 2H), 1.46-1.31 (m, 3H).

### Example 24: Compound 37

Compound 37: To a solution of compound 35 (24 mg, 46 μmole) and compound 2 (94 mg, 0.12 mmole) in of MeOH (1 mL) and water (1 mL) was added a solution of CuSO₄-THPTA (0.04M, 0.23 mL, 20 μmole) and sodium ascorbate (2.7 mg, 14 μmole) successively. The resulting solution was stirred for 3 days at room temperature. The solution was concentrated under reduced pressure and the mixture of mono- and di-coupled products was separated by C-18 column (water/MeOH, water only—1/4, v/v) To complete the reaction, this mixture was re-subjected to the reaction conditions as described above overnight at 40° C. The reaction solution was then dialyzed against water with dialysis tube MWCO 1000 while distilled water was changed every 6 hours. The aqueous solution in the tube was collected and lyophilized to give compound 37 as a white solid (53 mg, 55% yield).

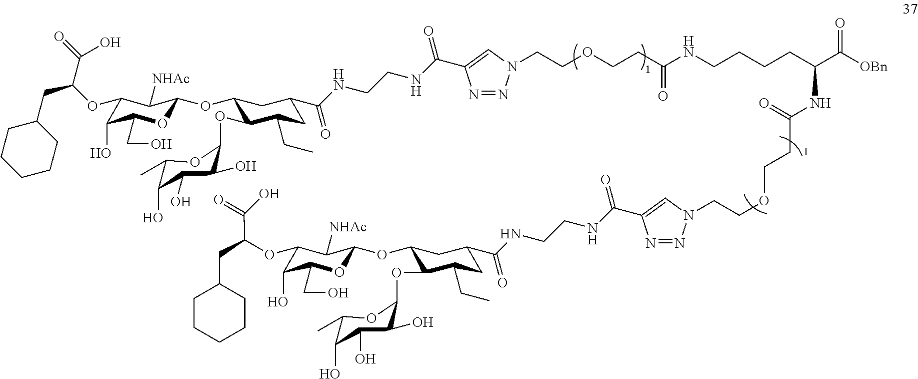

¹H NMR (400 MHz, Deuterium Oxide) δ 8.27 (broad two s, 2H), 7.27 (m, 5H), 5.05 (broad s, 2H), 4.92 (broad s, 2H), 4.81 (m, 2H), 4.62-4.28 (m, 6H), 4.20 (m, 1H), 4.09-3.55 (m, 26H), 3.55-3.10 (m, 13H), 2.93 (broad t, 2H), 2.42 (broad t, 2H), 2.31 (broad t, 2H), 2.20 (m, J=12.6 Hz, 2H), 2.06 (m, 4H), 1.95 (m, 10H), 1.84-1.36 (m, 12H), 1.35-0.91 (m, 12H), 0.91-0.72 (m, 10), 0.71-0.60 (broad t, 8H) MS: Calculated (C₉₇H₁₅₂N₁₄O₃₆, 2089.0), ES-Negative (2088.6, M−1, 1042.9 M/2-1).

### Example 25: Compound 38

Compound 38: A solution of compound 37 (13 mg, 6.2 μmole) in anhydrous MeOH (2 mL) was hydrogenated in the presence of Pd(OH)₂ (10 mg) for 2 hrs at room temperature. The solution was filtered through a Celite pad and the filtrate was concentrated. The crude product was purified by HPLC. The product portion was collected, evaporated, then lyophilized overnight to give compound 38 as a white solid (4.5 mg, 36% yield).

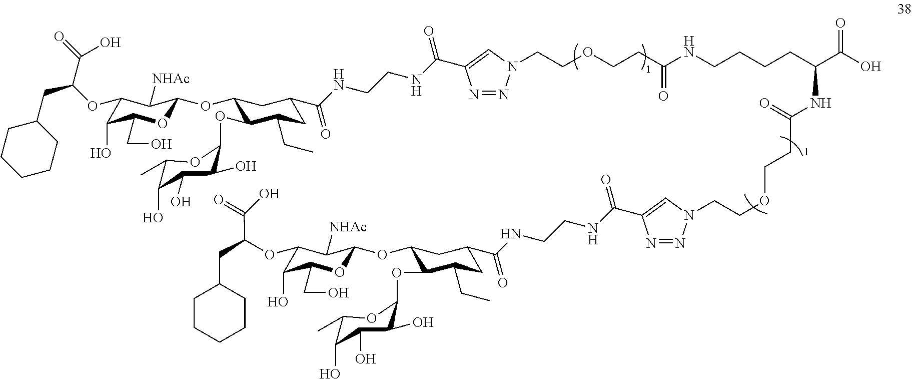

¹H NMR (400 MHz, Deuterium Oxide) δ 8.28 (two s, 2H), 4.89 (d, J=4.0 Hz, 2H), 4.79 (q, J=6.7 Hz, 2H), 4.54 (q, J=4.6 Hz, 5H), 4.40 (d, J=8.6 Hz, 2H), 3.98 (dd, J=8.5, 4.7 Hz, 1H), 3.95-3.79 (m, 6H), 3.78-3.74 (m, 5H), 3.73-3.67 (m, 6H), 3.66-3.55 (m, 13H), 3.54-3.32 (m, 11H), 3.31-3.24 (m, 2H), 3.18 (t, J=9.7 Hz, 2H), 2.92 (t, J=6.9 Hz, 2H), 2.49-2.33 (m, 2H), 2.30 (t, J=5.8 Hz, 2H), 2.19 (broad t, J=12.6 Hz, 2H), 2.11-1.98 (m, 2H), 1.92 (d, J=3.1 Hz, 6H), 1.79-1.33 (m, 24H), 1.23 (m, 3H), 1.18-0.89 (m, 20H), 0.88-0.69 (m, 5H), 0.64 (t, J=7.4 Hz, 6H) MS: Calculated (C₉₀H₁₄₆N₁₄O₃₆, 1999.0), ES-Negative (1219.2 M/2-1).

### Example 26: Compound 39

Compound 39: Compound 39 was prepared in 52% yield using an analogous procedure starting from compound 2 and compound 36.

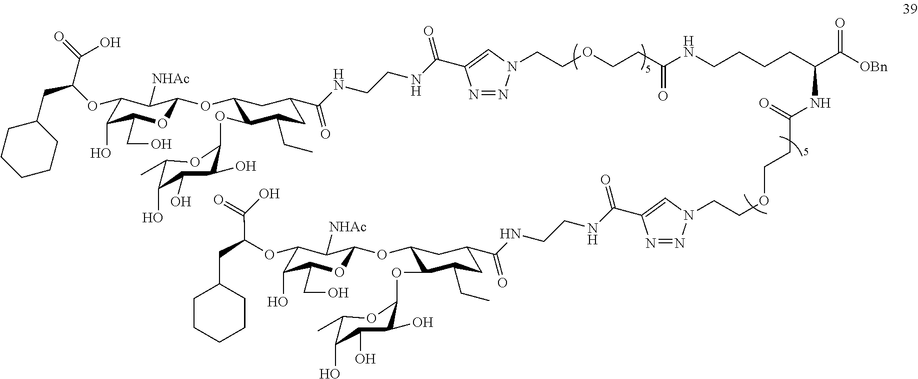

¹H NMR (400 MHz, Deuterium Oxide) δ 8.35 (s, 2H), 7.41-7.20 (m, 5H), 5.21-5.01 (dd, 2H), 4.92 (d, J=4.0 Hz, 2H), 4.81 (m, J=6.8 Hz, 2H), 4.58 (t, J=4.9 Hz, 4H), 4.42 (d, J=8.6 Hz, 2H), 4.35-4.21 (m, 1H), 3.88 (m, J=5.0 Hz, 6H), 3.84-3.75 (m, 5H), 3.74-3.70 (m, 4H), 3.69-3.59 (m, 11H), 3.58-3.44 (m, 36H), 3.43-3.34 (m, 6H), 3.33-3.24 (m, 3H), 3.20 (t, J=9.7 Hz, 2H), 3.03 (t, J=6.8 Hz, 2H), 2.46 (t, J=6.1 Hz, 2H), 2.37 (t, J=6.0 Hz, 2H), 2.20 (broad t, J=12.3 Hz, 2H), 2.05 (m, 2H), 1.93 (s, 6H), 1.82-1.33 (m, 24H), 1.32-1.18 (7H), 1.17-0.91 (m, 17H), 0.90-0.72 (m, 5H), 0.67 (t, J=7.3 Hz, 6H) MS: Calculated (C₁₁₃H₁₈₄N₁₄O₄₄, 2441.2), ES-Negative (1219.2 M/2-1).

### Example 27: Compound 40

Compound 40: Compound 40 was prepared in 26% yield using an analogous procedure starting from compound 39.

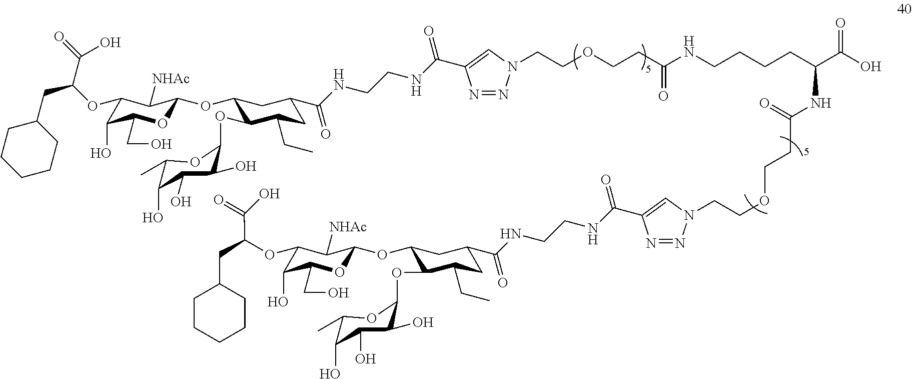

¹H NMR (400 MHz, Deuterium Oxide) δ 8.36 (two s, 2H), 4.90 (d, J=3.9 Hz, 2H), 4.80 (q, J=6.7 Hz, 2H), 4.58 (t, J=4.9 Hz, 5H), 4.41 (d, J=8.6 Hz, 2H), 4.05 (dd, J=8.5, 4.7 Hz, 1H), 3.98-3.82 (m, 5H), 3.81-3.72 (m, 5H), 3.72-3.59 (m, 11H), 3.59-3.32 (m, 34H), 3.32-3.11 (m, 5H), 3.06 (t, J=6.9 Hz, 2H), 2.57-2.42 (m, 2H), 2.38 (t, J=6.1 Hz, 2H), 2.20 (broad t, J=12.2 Hz, 2H), 2.04 (m, 2H), 1.92 (s, 6H), 1.78-1.32 (m, 20H), 1.32-0.88 (m, 24H), 0.89-0.70 (m, 5H), 0.66 (t, J=7.3 Hz, 6H) MS Calculated (C₁₀₆H₁₇₈N₄O₄₄, 2351.2), ES-negative (1173.9 M/2-1, 782.3, M/3−1).

### Example 28: Compound 42

Compound 42: A solution of compound 41 (described in JACS, 2002, 124(47), 14085) (22 mg, 49 μmole) and DIPEA (28 μL, 163 μmole) in anhydrous DMF (0.3 mL) was cooled to 0° C. and HATU (62 mg, 163 μmole) was added. The solution was stirred for 30 minutes. This solution was added to a solution of compound 1 (0.12 g, 163 μmole) over a 5 min. period. The resulting solution was stirred overnight. The reaction solution was dialyzed against water with dialysis tube MWCO 1000 while distilled water was changed every 6 hours. The aqueous solution in the tube was collected and lyophilized overnight to give compound 42 as a white solid (69 mg, 54%).

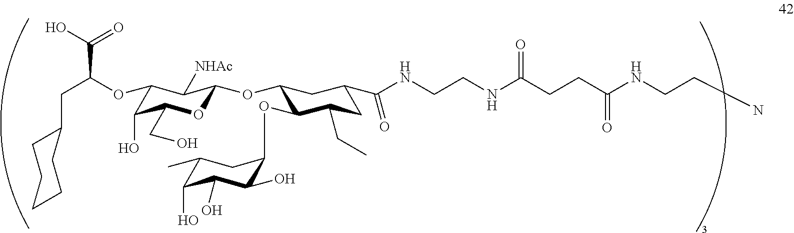

¹H NMR (400 MHz, Methanol-d₄) δ 4.85 (m, 6H), 4.52 (broad s, 3H), 3.75 (M, J=11.1 Hz, 15H), 3.69-3.52 (m, 12H), 3.38 (broad t, J=1.7 Hz, 3H), 3.37-3.06 (m, 45H, partially hidden by MeOH), 2.67 (m, 6H), 2.52-2.34 (m, 15H), 2.15 (broad t, 3H), 2.08-1.96 (m, 3H), 1.88 (m, 12H), 1.73 (m, 3H), 1.70-1.37 (m, 12H), 1.36-0.98 (m, 36H), 0.93-0.71 (m, J=7.3 Hz, 15H). MS: Calculated for C₁₂₀H₂₀₁N₁₃O₄₈=2592.3; Found ES-Negative m/z=1295.6 (M/2−H).

### Example 29: Compound 44

Compound 44: A solution of tetravalent PEG-active ester (Average MW=20176, 0.5 g, 0.24 mmole) in DMSO (5 mL) was added a solution of compound 1 (1.4 g, 1.93 mmole) and DIPEA (0.5 mL) in distilled water (10 mL) over 1 hr period at room temperature. The resulting solution was stirred for 3 days under the same condition. The reaction solution was dialyzed against water with dialysis tube MWCO 1000 while distilled water was changed every 6 hours. The aqueous solution in the tube was collected and lyophilized overnight to give compound 44 (average chain length (n)=110) as a white solid (0.67 g, 0.15 mmole, 63%).

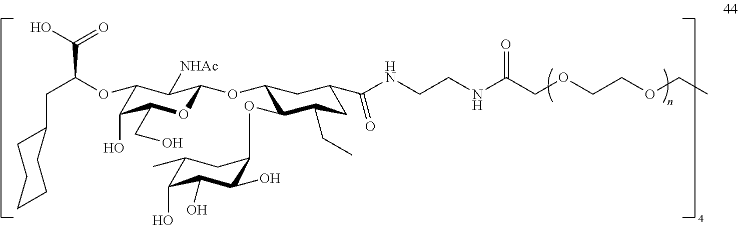

¹H NMR (400 MHz, Deuterium Oxide) δ 4.92 (d, J=4.0 Hz, 4H), 4.81 (d, J=6.8 Hz, 4H), 4.42 (d, J=7.8 Hz, 4H), 3.96 (s, 8H), 3.78 (m, 12H), 3.74-3.50 (m, 188H), 3.42-3.34 (m, 12H), 3.33-3.16 (m, 8H), 3.10 (q, J=7.4 Hz, 4H), 2.37-2.14 (m, 4H), 2.05 (m, 4H), 1.96 (s, 12H), 1.75 (m, 8H), 1.70-1.33 (m, 8H).

### Example 30: Compound 45

Compound 45: A solution of compound 32 (300 mg, 0.2 mmole) and DIPEA (0.2 mL, 1.0 mmole) in anhydrous DMF (15 mL) was cooled to 0° C. TBTU (200 mg, 0.6 mmole) was added. The resulting solution was stirred for 3 hrs at room temperature. Azetidine (4.0 mL, 60.0 mmol) was added. The solution was transferred to a sealed tube and stirred overnight at 55° C. The reaction mixture was cooled to room temperature and concentrated in vacuo. The residue was partially purified by chromatography using the Combi-flash system and eluting with EtOAc/MeOH/water (5/5/1, v/v/v). The crude product was de-salted using a C-18 column (water/MeOH, 9/1-1/9, v/v). The pure product was lyophilized to afford a white solid (0.37 g, 2.35 mmole, quantitative).

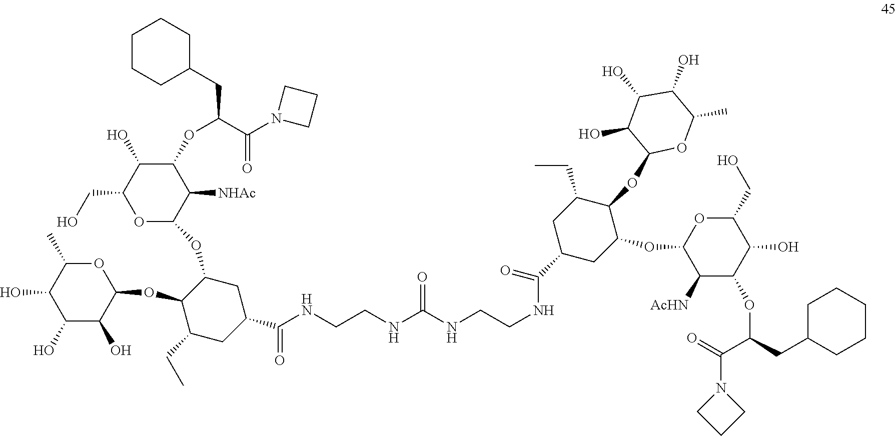 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](O[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

¹H NMR (400 MHz, Deuterium Oxide) δ 4.93 (broad s, 1H), 4.88-4.76 (m, 1H), 4.42 (broad s, 1H), 4.19 (m, 3H), 3.97 (m, 3H), 3.88-3.73 (m, 2H), 3.72-3.54 (m, 6H), 3.42 (m, 2H), 3.29-3.00 (m, 6H), 2.67-2.49 (m, 0.5H), 2.35-2.15 (m, 4H), 2.14-1.98 (m, 1H), 1.94 (s, 3H), 1.75 (broad d, J=12.8 Hz, 2H), 1.68-1.36 (m, 8H), 1.35-1.17 (m, J=11.3 Hz, 4H), 1.16-0.98 (dd, J=20.5, 9.1 Hz, 7H), 0.94-0.67 (m, J=32.9, 8.9 Hz, 5H) MS: Calculated (C75H126N8O27, 1570.8), ES-Positive (1594.5, M+Na; 808.5 (M/2+Na), ES-Negative (1569.6, M−1; 784.4, M/2-1).

### Example 31

### E-Selectin Activity—Analysis by SPR

Surface Plasmon Resonance (SPR) measurements were performed on a Biacore X100 instrument (GE Healthcare). A CM5 sensor chip (GE Healthcare) was used for the interaction between E-selectin and GMI compound. Anti-human IgG (Fc) antibody (GE Healthcare) was immobilized onto the chip by amine coupling according to the manufacturer's instructions. In brief, after a 7-min injection (flow rate of 5 μl/min) of 1:1 mixture of N-ethyl-N′-(3-dimethylaminopropyl) carbodiimide hydrochloride and N-hydroxysuccinimide, anti-human IgG (Fc) antibody (25 μg/ml in 10 mM sodium acetate buffer, pH 5.0) was injected using a 6-min injection at 5 μl/min. Remaining activated groups were blocked by injecting 1 M ethanolamine/HCl, pH 8.5. The recombinant human E-selectin/CD63E Fc Chimera (50 μg/ml) (R & D systems) was injected into the experimental cell until 6000-7000 RU was captured onto the antibody surface. No recombinant human E-selectin/CD63E was injected into the control cell. Increasing concentrations of GMI compound samples were injected at 30 μl/min into both flow cells and all sensorgrams were recorded against the control. Regeneration of the anti-human IgG (Fc) surface was achieved by injecting 3M magnesium chloride, followed by 50 mM sodium hydroxide. Data were analyzed using Biacore X100 evaluation/BIA evaluation 4.1.1 software (GE Healthcare) and Graphad prism 6 software.

E-Selectin Antagonist Activity of Compounds

| Compound | KD (nM) |
|---|---|
| 11 | 8.5 |
| 12 | 3.7 |
| 13 | 4.7 |
| 14 | 6.6 |
| 15 | 6.0 |
| 16 | 6.0 |
| 17 | 5.1 |
| 18 | 3.5 |
| 19 | 10.2 |
| 20 | 0.8 |
| 21 | 18.3 |
| 22 | 2.3 |
| 23 | 2.8 |
| 24 | 2.4 |
| 25 | 3.0 |
| 27 | 8.6 |
| 28 | 3.2 |
| 29 | 3.2 |
| 30 | 2.0 |
| 31 | 3.3 |
| 32 | 8.8 |
| 37 | 3.9 |
| 38 | 6.4 |
| 39 | 5.1 |
| 40 | 5.5 |
| 42 | 1.5 |
| 44 | 8.0 |
| 45 | 2.1 |
| 3 (monomer —CO₂H) | 2260 |
| 4 (monomer azetidine) | 2600 |

### Example 32

### Study Testing the Effect of Compound 45 on Sickle Cell Vaso-Occlusion

### Test Article and Vehicle

Description, Identification and Storage: The active compound, Compound 45, as a powder provided by GlycoMimetics was stored at −20° C. as recommended by GlycoMimetics, and vehicle saline.

Preparations: A stock solution of Compound 45 is dissolved in saline is prepared weekly and stored at −20° C.

### Test Systems/Method Descriptions

Two distinct murine models of sickle cell disease were used in this project and each model is describe fully below, first for a nude mouse system and secondly for the Townes transgenic mouse system.

### Nude Mouse Model:

Animals (species, strain and supplier): Nude (nu−/nu−) mice (male and female) 8-12 weeks of age and obtained from Duke University were used in this project.

Animal work was approved by the Institutional Animal Care and Use Committee (IACUC) at Duke University, and experiments were carried out in accordance with the National Institutes of Health (NIH) guidelines and recommendations for the Care and Use of Laboratory Animals. In these studies, we used half male and half female nude mice.

Rationale of Using the Nude Mouse Model: The nude mouse model is an excellent model to study the pathophysiology of SCD, because these mice allow the use of human sickle RBCs in the presence of all physiological parameters, and the presence of normal blood flow, except the RBCs. For this reason, Nude mice are probably one of the most relevant models for studying the efficacy and mode of action of drug candidates specifically on sickle RBCs and their interactions with different cell types.

### Methodology

In vivo treatment: Mice were divided into 3 groups. All mice were first injected I.P. with 500 ng TNFα to induce inflammation, activate the endothelium, and allow adhesion of murine leukocytes. After 2 hours, and once the endothelium is activated and murine leukocytes have already adhered, mice were infused with 200 μl of DIL (rhodamine)-labeled human sickle RBCs at 50% hematocrit (less than 10% murine blood volume assuming that blood volume for a 20 g weight mouse is 1.5 ml). We injected I.V. the three animal groups with the first dose of either Compound 45 at 20 μg/kg or 40 μg/kg, or saline (10 mL/kg) as vehicle immediately after infusion of human RBCs. The second dose was given 30 minutes later.

### In Vivo Experimental Design with Nude Mice in Table Format

Animals

| Animal | | |
|---|---|---|
| Species | Mice | |
| Strain | Nude Mice (nu-/nu-) | |
| Age | 8-12 weeks | |
| Quantity | Nude mice: half females/half | |
| | males per treatment condition | |
| | Total to be tested: 24 nude mice | |
| Treatment groups | VOC Study | |
| Strain | Treatment | Animals/group |
| Nude mice | Vehicle I.V. | 8 |
| Nude mice | 40 μg/kg; Cmpd. 45; I.V. | 8 |
| Nude mice | 80 μg/kg; Cmpd. 45; I.V. | 8 |

Test material, Dose, Application route, Volume and Formulation

| Vehicle | Saline |
|---|---|
| Dose volume | 10 mL/kg |
| Application route | I.V. |
| Frequency | 1^{st} dose immediately after human RBCs |
| | infusion |
| | 2^{nd} dose 30 min later |

Test item

| Active Compound | Compound 45 |
|---|---|
| Total Dose | 40, and 80 μg/kg |
| Dose volume | 10 mL/kg |
| Application route | I.V. |
| Frequency | 1^{st} dose immediately after human SSRBCs |
| | infusion |
| | 2^{nd} dose 30 min later |

Window chamber surgery: Surgery was performed before TNFα administration. General anesthesia was achieved by exposing nude mice to isoflurane. A double-sided titanium frame window chamber was surgically implanted into the dorsal skin fold under sterile conditions using a laminar flow hood. Surgery involved carefully removing the epidermal and dermal layers of one side of a dorsal skin fold, exposing the blood vessels of the subcutaneous tissue adjacent to the striated muscles of the opposing skin fold, and then securing the two sides of the chamber to the skin using stainless steel screws and sutures. A glass window was placed in the chamber to cover the exposed tissue and secured with a snap ring.

Animal treatments, and fluorescence intravital microscopy: Anesthetized nude mice were first injected with 500 ng TNFα. After two hours, mice were infused with 200 μl DIL (rhodamine)-labeled human sickle RBCs at 50% hematocrit DIL through the dorsal tail vein.

Immediately after human RBC injection, nude mice were then injected I.V. with the first dose of either Compound 45 at 20 μg/kg (n=4) or 40 μg/kg (n=3), or saline (10 mL/kg) as vehicle (n=5). Videos were recorded for 30 minutes at different locations within the dorsal skin-fold window chamber to accurately determine the effects of Compound 45 on human sickle cell adhesion to the vascular endothelium and adherent murine leukocytes, and vaso-occlusion in the nude mice. Thirty minutes later, the second dose of either Compound 45 at 20 μg/kg (n=4) or 40 μg/kg (n=3), or vehicle (n=5) was injected I.V., and intravital microscopy measurements of blood cell flow dynamics in post-capillary venules and arterioles were resumed for another 30 minutes on anesthetized nude mice. Videos were again recorded at different locations within the dorsal skin-fold window chamber to accurately determine the effects of the second dose of Compound 45 on blood cell adhesion to the vascular endothelium, and vaso-occlusion. Videos were produced using 10× and 20× magnifications.

Cell adhesion was quantified on still images by measuring the fluorescence intensity [fluorescence unit (FU)] of adherent fluorescence-labeled human sickle cells using ImageJ software downloaded from the NIH website. Blood flow was also determined by counting the number of vessels with normal blood flow, slow blood flow, and no blood flow by frame-by-frame analysis of video replay. The values were averaged among groups of animals (n=5 for vehicle, n=4 for 40 μg/kg total Compound 45, and n=3 for 80 μg/kg total Compound 45) for non-blinded statistical analysis.

Flow cytometry analysis of circulating human sickle RBCs: Following intravital microscopy, blood samples were collected from nude mice treated with vehicle (n=3), 40 μg/kg total Compound 45 (n=3), and 80 μg/kg total Compound 45 (n=2), through cardiac puncture into EDTA tubes to determine the number of circulating human sickle RBCs by flow cytometry.

### Data Analysis and Statistics

Endpoints Measured: Endpoints measured included (1) number of circulating human sickle RBCs and (2) cell adhesion in FU, and blood flow.

Method of Analysis: Human sickle RBC adhesion is presented as FU, blood flow as % normal blood flow, % slow blood flow and % no blood flow (or occluded vessels), and circulating human sickle RBCs as number of circulating sickle RBCs.

The values were averaged among the number of animals. All measurements were recorded, and results were saved. Raw results were exported to excel files and will be provided as a separate file. Data were also presented using Prism files, and for further data processing and analysis.

Statistics: Data were compared using parametric analyses (GraphPad Prism 5 Software), including repeated and non-repeated measures of analysis of variance (ANOVA). One-way ANOVA analyses were followed by Bonferroni corrections for multiple comparisons (multiplying the p value by the number of comparisons). A p value<0.05 is considered significant.

### Results

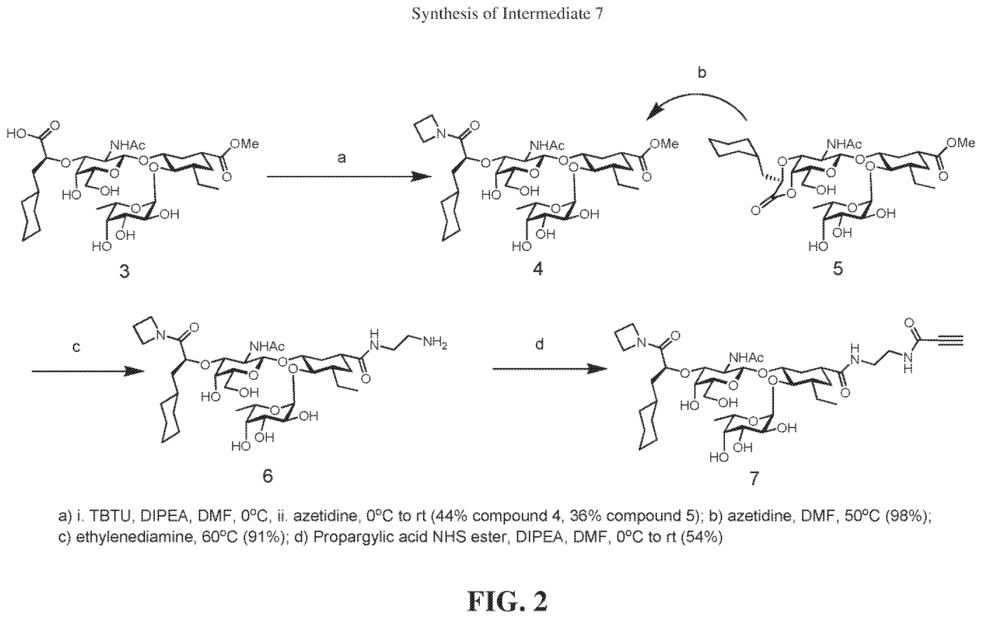

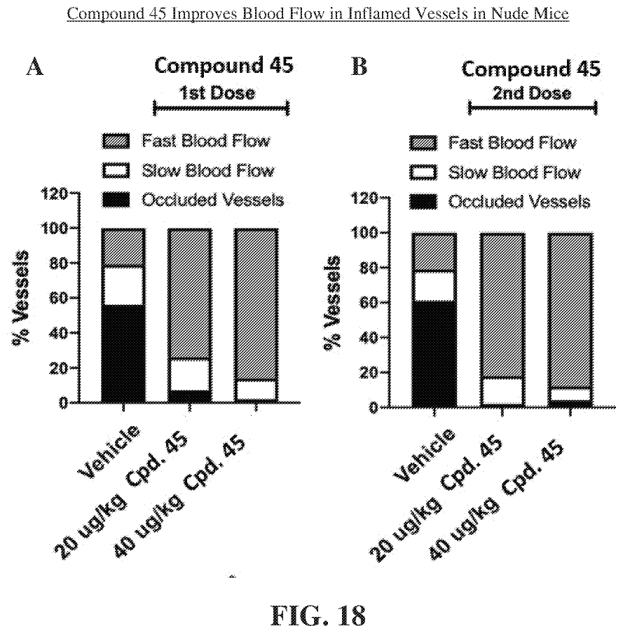

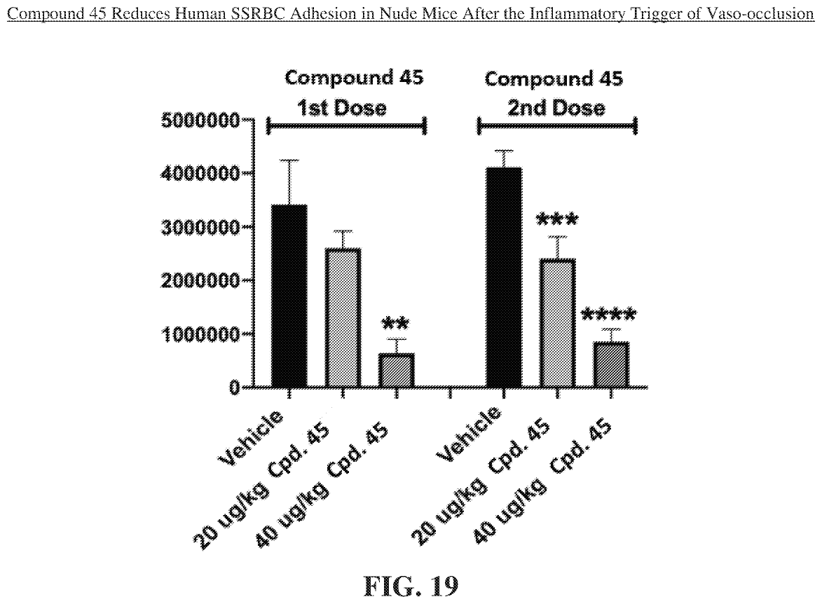

Compound 45 decreased blood cell adhesion and prevented vaso-occlusive crisis. Vaso-occlusion in response to inflammation is one of the major pathophysiologic processes in SCD. In nude mice, we have previously shown that human sickle RBCs bind to both adherent murine leukocytes and the vascular endothelium in inflamed venules producing vaso-occlusion. See Zennadi R, Moeller B J, Whalen E J, et al. “Epinephrine-induced activation of LW-mediated sickle cell adhesion and vaso-occlusion in vivo” Blood 2007; 110(7):2708-2717. We assessed whether the inhibitor of E selectin, Compound 45, reduces human sickle cell adhesion, and prevents the progression of a vaso-occlusive crisis (VOC) event in TNFα-treated nude mice in vivo. Continuous intravital microscopy observations of the enflamed microvasculature of nude mice treated with the first dose of vehicle, for an approximate period of 30 minutes, showed marked adhesion of human sickle RBCs (FIG. 19), and occluding 56% of the total vessels recorded (FIG. 18A). Cell adhesion in inflamed venules was persistent after injecting the second dose of vehicle in nude mice 30 minutes later, occluding 61% of the total micro-vessels recorded with evident blood stasis (FIG. 18B), which was irreversible for the whole period (1 hour) of the intravital microscopy. In 18% of the total vessel segments, blood flow was sluggish and slow in this mouse group treated with vehicle (FIG. 18B). While one dose of Compound 45 at 20 μg/kg failed to reduce SSRBC adhesion compared to the vehicle group, adhesion of the sickle cells was significantly diminished following infusion of the second dose of 20 μg/kg in nude mice (FIG. 1). As a result of this positive outcome, the percentage of the total occluded vessel segments was reduced to 2% (FIG. 2B). However, the total venules with slow blood flow (16%) was almost comparable to the vehicle treatment (18%) (FIG. 18B). In sharp contrast, both one dose of 40 μg/kg and two doses of 40 μg/kg injected at 30 minutes interval had marked anti-SSRBC adhesive activity (FIG. 19). This effect on cell adhesion was stronger than that of 20 μg/kg Compound 45 whether the nude mice had received the first dose of the compound or following the second dose (FIG. 19). Reduction in the number of adherent SSRBCs by Compound 45 after the first and the two doses of 40 μg/kg was also reflected by the significant decrease in the percentage of obstructed total vessels (2% and 4% of the total venules recorded, respectively), and vessels with slow blood flow (12% and 8% of the total vessels recorded, respectively) (FIGS. 18A and 18B).

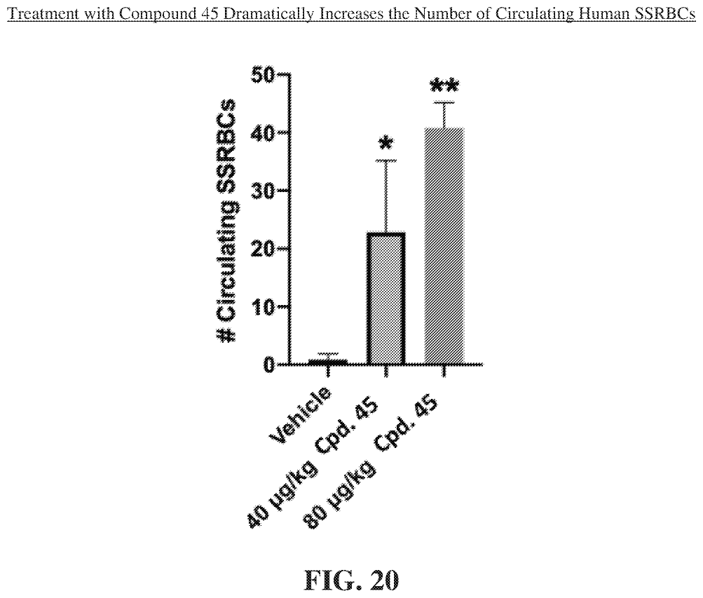

As a result of reduced adhesion and VOC in nude mice treated with two doses of 20 μg/kg and 40 μg/kg at 30 minutes interval of Compound 45, the number of circulating human SSRBCs increased significantly compared to animals treated with vehicle (FIG. 20). These data strongly suggest that Compound 45 can prevent inflammation from exacerbating VOC by at least down-regulating adhesive function of SSRBCs via inhibition of their binding to E selectin exposed on the vascular endothelium, and possibly activated Mac-1 on leukocytes.

### Summary of the Results in Nude Mice

The E selectin inhibitor, Compound 45, particularly at 40 μg/kg given twice (80 μg/kg total) to nude mice, once immediately after human sickle RBC infusion, and once 30 min later, was effective in reducing cell adhesion in venules and vaso-occlusion and restoring blood flow. These effective and beneficial anti-adhesive effects were a result of inhibition with Compound 45 of E selectin involved in adhesion of human sickle RBCs to the endothelium, and adherent leukocytes following the inflammatory response to TNFα.

### Townes Mouse Model:

Animals (species, strain and supplier): HbSS-Townes mice on a 129/B6 background (Jackson Laboratory, Bar Harbor, Me., USA, 10-12 weeks old) and maintained at Duke University were used in this project.

Animal work was approved by the Institutional Animal Care and Use Committee (IACUC) at Duke University, and experiments were carried out in accordance with the National Institutes of Health (NIH) guidelines and recommendations for the Care and Use of Laboratory Animals.

Rationale of Using the Townes Mouse Model: Townes mice have a transgene containing normal human α, γ, δ globins and sickle β globin and targeted deletions of murine α & β globins (α^{-/-}, β^{-/-}, Tg^{SS}). This mouse model of SCD expresses exclusively human sickle hemoglobin. Townes sickle mice are used in this project because they have baseline inflammation, RBC oxidative damage and endothelial abnormalities, all predisposing to a more severe vaso-occlusion phenotype, better reflecting human SCD pathophysiology in people. In addition, these mice have essentially 100% sickle RBCs. The Townes mouse model is an excellent model to study the pathophysiology of sickle cell disease, because these mice represent a severe end of the sickle cell disease spectrum. For this reason, these mice are probably the most sensitive system in which to test for the mechanistic effects of a variety of sickle cell abnormalities. In addition, the Townes sickle mice tolerate well surgery of window chamber implants. Furthermore, Rahima et al. have shown that the Townes sickle mice exposed to TNFα for 2 hours were highly sensitive to these conditions and became lethargic but survived. Importantly, the blood flow velocity was significantly reduced in these sickle mice but not in control wild type mice subjected to these conditions.

### Methodology

In vivo treatment: Townes mice were divided into 3 groups. All mice were first injected I.V. with PE-anti-mouse TER-119 antibody (10 μg/g BW) to label RBCs. Thirty minutes later, animals are injected with 500 ng TNFα I.P. to induce vaso-occlusion. After 90 minutes, and after onset of vaso-occlusion, sickle mice were treated I.V. with the first dose of either Compound 45 at 20 μg/kg or 40 μg/kg, or saline (10 mL/kg) as vehicle. The second dose was given 30 minutes later.

### In Vivo Experimental Design with Townes Mice in Table Format

Animals

| Animal | | |
|---|---|---|
| Species | Mice | |
| Strain | HbSS-Townes mice on a 129/B6 background | |
| Age | 8-12 weeks | |
| Quantity | Townes mice: females | |
| | Total to be tested: 9 Townes mice | |
| Treatment groups | VOC Study | |
| Strain | Treatment | Animals/group |
| Townes mice | Vehicle I.V. | 3 |
| Townes mice | 40 μg/kg; Cmpd. 45; I.V. | 3 |
| Townes mice | 80 μg/kg; Cmpd. 45; I.V. | 3 |

| Test material, Dose, Application route, | |
|---|---|
| Volume and Formulation | |
| Vehicle | Saline |
| Dose volume | 10 mL/kg |
| Application route | I.V. |
| Frequency | 1^{st} dose 90 minutes post TNF injection |
| | 2^{nd} dose 30 min later |

Test item

| Active Compound | Compound 45 |
|---|---|
| Total Dose | 40, and 80 μg/kg |
| Dose volume | 10 mL/kg |
| Application route | I.V. |
| Frequency | 1^{st} dose 90 minutes post TNF injection |
| | 2^{nd} dose 30 min later |

Window chamber surgery: For Townes mice experiments, surgery is carried out under sterile conditions with aseptic technique. Since these mice do have hair, it is necessary to shave and use hair removal cream on the back of the anesthetized mouse prior to cleaning the back of the animals, and performing surgery. Experimental studies are performed immediately after surgery, because in our experience, window chambers are not usable a few days after surgery. The skin is very dry, and blood cells cannot be visualized under the microscopy. In addition, sickle mice have been reported to have a mild inflammatory response to the dorsal skin-fold window chamber implantation, evidenced by elevated levels of serum amyloid P component (SAP) 3 days after surgery.

Intravital microscopy: Anesthetized animals with window chambers were placed on the stage of an Axoplan microscope (Carl Zeiss, Thornwood, N.Y.) and temperature maintained at 37° C. using a thermostatically controlled heating pad. Labeled RBC and leukocyte adhesion, and blood flow dynamics were observed in subdermal vessels for at least 30 minutes using 20× and 10× magnifications. Microcirculatory events and cell adhesion were simultaneously recorded using a computer connected to a digital video camera C2400 (Hamamatsu Photonics K.K., Japan). Visible venules were examined for each set of conditions. Arterioles were distinguished from venules based on: 1) observation of divergent flow as opposed to convergent flow; 2) birefringent appearance of vessel walls using transillumination, which is characteristic of arteriolar vascular smooth muscle; and 3) relatively straight vessel trajectory without evidence of tortuosity.

### Data Analysis and Statistics

Data Analysis and Statistics were conducted as described above for the nude mouse model.

### Results

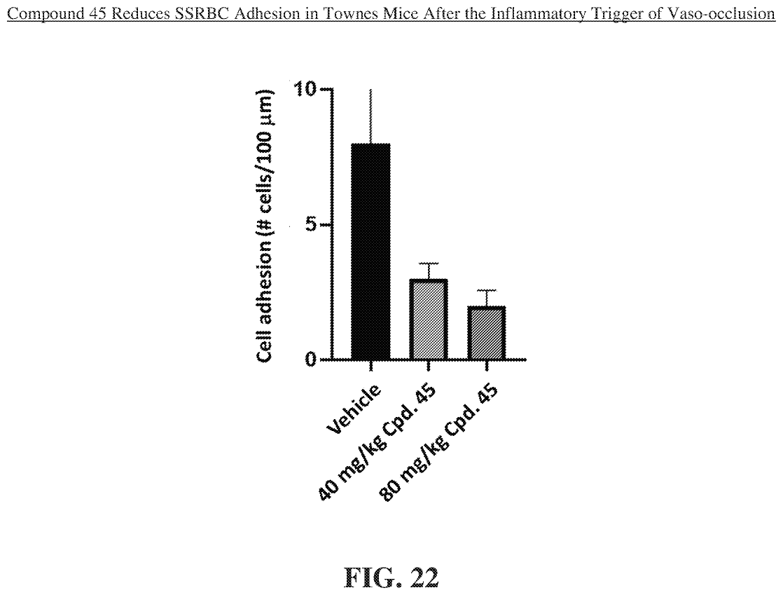

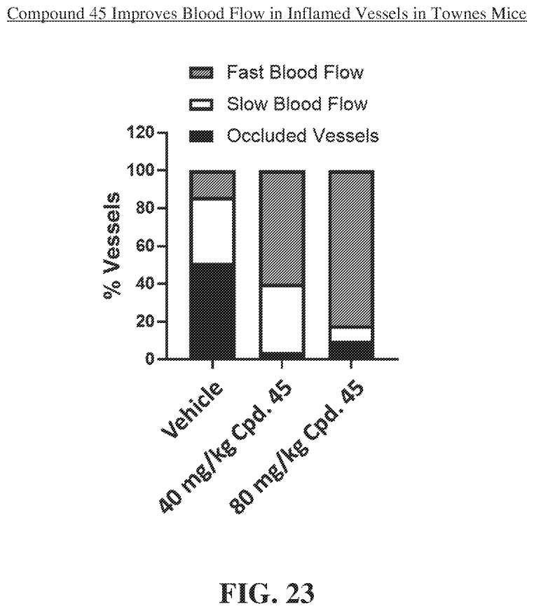

Compound 45 decreased blood cell adhesion and prevented vaso-occlusive crisis in the Townes mouse model. We assessed whether the inhibitor of E selectin, Compound 45, reduces sickle cell adhesion, and prevents the progression of a vaso-occlusive crisis (VOC) event in TNFα-treated Townes mice in vivo. Continuous intravital microscopy observations of the enflamed microvasculature of nude mice treated with two injections of vehicle showed marked adhesion of sickle RBCs (FIG. 22), and occluding approximately 50% of the total vessels recorded (FIG. 23). In approximately 30% of the total vessel segments, blood flow was sluggish and slow in this mouse group treated with vehicle (FIG. 23). Following administration with two 20 μg/kg doses or two 40 μg/kg doses of Compound 45, adhesion of the sickle cells was significantly diminished (FIG. 22). As a result of this positive outcome, the percentage of the total occluded vessel segments was reduced by 80-90% (FIG. 23).

### Summary of the Results

The E selectin inhibitor, Compound 45, at 40 μg/kg given twice (80 μg/kg total) to Townes mice, once 90 minutes after TNFα injection and once 30 min later, was effective in reducing sickle RBCs adhesion in venules and vaso-occlusion and restoring blood flow. Similar to our observations in the nude mouse model of SCD, these effective and beneficial anti-adhesive effects were a result of inhibition with Compound 45 of E selectin involved in adhesion of human sickle RBCs to the endothelium, and adherent leukocytes following the inflammatory response to TNFα.

### CONCLUSIONS

Our preliminary data demonstrate that Compound 45 injected intravenously to nude mice at 20 μg/kg twice at 30 minutes interval, or once at 40 μg/kg immediately after infusion of human SSRBCs or twice at also 30 minutes interval reduced adhesion of human SSRBCs in enflamed vascular endothelium, and VOC. As a result, SSRBC microcirculatory behavior improved, and normal blood flow was restored in most of the vessel segment. These improved events led to increased SSRBC counts. Similar results on the attenuation of sickle red cell adhesion and venule occlusion were obtained following administration of Compound 45 in the Townes transgenic mouse model. Compound 45 is a valuable therapeutic compound able to treat acute VOC episodes in SCD.

### REFERENCES

The following references are hereby incorporated by reference in their entirety.
Knight-Madden J, Vergani D, Patey R, et al. Cytokine levels and profiles in children related to sickle cell disease and asthma status. Journal of interferon & cytokine research: the official journal of the International Society for Interferon and Cytokine Research. 2012; 32(1):1-5.
Musa B O, Onyemelukwe G C, Hambolu J O, Mamman A I, Isa A H. Pattern of serum cytokine expression and T-cell subsets in sickle cell disease patients in vaso-occlusive crisis. Clinical and vaccine immunology: CVI. 2010; 17(4):602-608.
Qari M H, Dier U, Mousa S A. Biomarkers of inflammation, growth factor, and coagulation activation in patients with sickle cell disease. Clinical and applied thrombosishemostasis: official journal of the International Academy of Clinical and Applied Thrombosis/Hemostasis. 2012:18(2):195-200.
Zennadi R, Moeller B J, Whalen E J, et al. Epinephrine-induced activation of LW-mediated sickle cell adhesion and vaso-occlusion in vivo. Blood 2007; 110(7):2708-2717.
Steinberg M H, Hebbel R P. Clinical diversity of sickle cell anemia: genetic and cellular modulation of disease severity. American journal of hematology. 1983; 14(4):405-416.
Steinberg M H, Lu Z H, Barton F B, Terrin M L, Charache S, Dover G J. Fetal hemoglobin in sickle cell anemia: determinants of response to hydroxyurea. Multicenter Study of Hydroxyurea. Blood. 1997; 89(3):1078-1088.
Neel J V. The Inheritance of Sickle Cell Anemia. Science. 1949; 110(2846):64-66.
Head C A, Brugnara C, Martinez-Ruiz R, et al. Low concentrations of nitric oxide increase oxygen affinity of sickle erythrocytes in vitro and in vivo. J Clin Invest. 1997; 100(5):1193-1198.
Hebbel R P, Moldow C F, Steinberg M H. Modulation of erythrocyte-endothelial interactions and the vasocclusive severity of sickling disorders. Blood. 1981; 58(5):947-952.
Brugnara C, De Franceschi L, Bennekou P, Alper S L, Christophersen P. Novel therapies for prevention of erythrocyte dehydration in sickle cell anemia. Drug news & perspectives. 2001; 14(4):208-220.
Wun T. The Role of Inflammation and Leukocytes in the Pathogenesis of Sickle Cell Disease, Hemoglobinopathy. Hematology. 2001; 5(5):403-412.
Osarogiagbon U R, Choong S, Belcher J D, Vercellotti G M, Paller M S, Hebbel R P. Reperfusion injury pathophysiology in sickle transgenic mice. Blood. 2000; 96(1):314-320.
Ryan T M, Townes T M, Reilly M P, et al. Human sickle hemoglobin in transgenic mice. Science. 1990; 247(4942):566-568.
Townes T M, Ryan T M, Behringer R R, Palmiter R D, Brinster R L. DNase I super-hypersensitive sites direct high level erythroid expression of human alpha-, beta- and beta s-globin genes in transgenic mice. Progress in clinical and biological research. 1989; 316A:47-61.

1. A method for the treatment of sickle cell disease or complications associated therewith, the method comprising administering to a subject in need thereof an effective amount of at least one compound chosen from glycomimetic E-selectin antagonists of Formula (I): 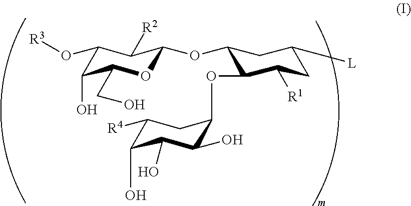
prodrugs of Formula (I), and pharmaceutically acceptable salts of any of the foregoing, wherein
each R¹, which may be identical or different, is independently chosen from H, C₁₋₁₂ alkyl, C₂₋₁₂ alkenyl, C₂₋₁₂ alkynyl, and —NHC(═O)R⁵ groups, wherein each R⁵, which may be identical or different, is independently chosen from C₁₋₁₂ alkyl, C₂₋₁₂ alkenyl, C₂₋₁₂ alkynyl, C₆₋₁₈ aryl, and C₁₋₁₃ heteroaryl groups;
each R², which may be identical or different, is independently chosen from halo, —OY¹, —NY¹Y², —OC(═O)Y¹, —NHC(═O)Y¹, and —NHC(═O)NY¹Y² groups, wherein each Y¹ and each Y², which may be identical or different, are independently chosen from H, C₁₋₁₂ alkyl, C₂₋₁₂ alkenyl, C₂₋₁₂ alkynyl, C₁₋₁₂ haloalkyl, C₂₋₁₂ haloalkenyl, C₂₋₁₂ haloalkynyl, C₆₋₁₈ aryl, and C₁₋₁₃ heteroaryl groups, wherein Y¹ and Y² may join together along with the nitrogen atom to which they are attached to form a ring;
each R³, which may be identical or different, is independently chosen from 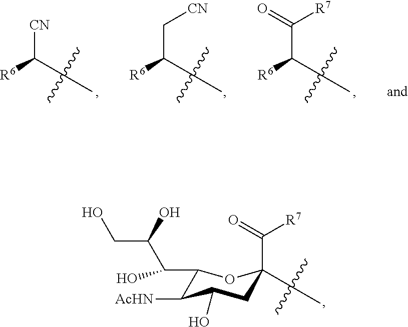
wherein each R⁶, which may be identical or different, is independently chosen from H, C₁₋₁₂ alkyl and C₁₋₁₂ haloalkyl groups, and wherein each R⁷, which may be identical or different, is independently chosen from C₁₋₈ alkyl, C₂₋₈ alkenyl, C₂₋₈ alkynyl, —OY³, —NHOH, —NHOCH₃, —NHCN, and —NY³Y⁴ groups, wherein each Y³ and each Y⁴, which may be identical or different, are independently chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups, wherein Y³ and Y⁴ may join together along with the nitrogen atom to which they are attached to form a ring;
each R⁴, which may be identical or different, is independently chosen from —CN, C₁₋₄ alkyl, and C₁₋₄ haloalkyl groups;
m is chosen from integers ranging from 2 to 256; and
L is chosen from linker groups.

2. The method according to claim 1, with the proviso that when m is 4, each R¹ and each R⁴ is methyl, each R² is —OC(═O)Ph, and each R³ is 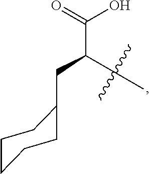 [SMILES: *[C@@H](CC1CCCCC1)C(=O)O]
then the linker groups are not chosen from 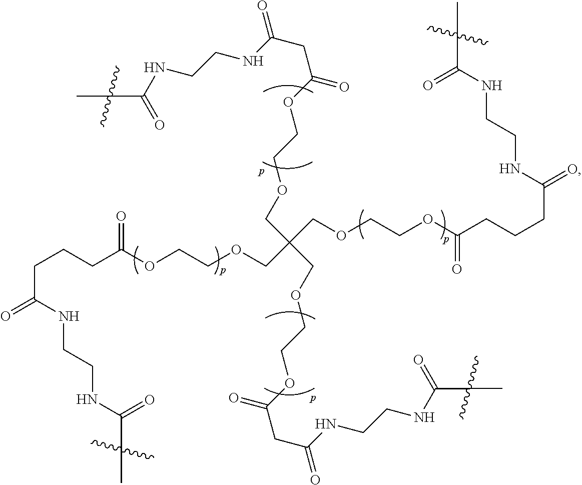
wherein p is chosen from integers ranging from 0 to 250.

3. The method according to claim 2, wherein at least one R¹ is H.

4. The method according to claim 2, wherein at least one R¹ is methyl.

5. The method according to claim 2, wherein at least one R¹ is ethyl.

6. The method according to claim 2, wherein at least one R¹ is chosen from —NHC(═O)R⁵ groups.

7. The method according to claim 6, wherein at least one R⁵ is chosen from 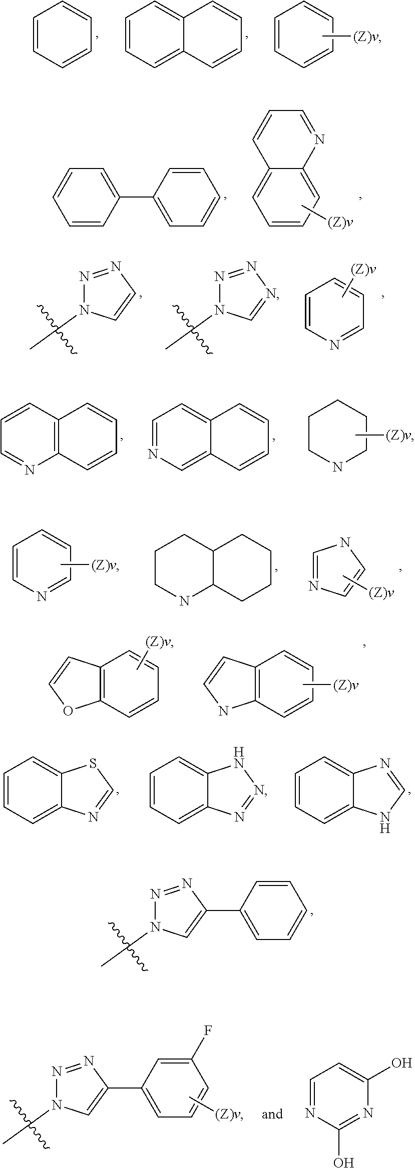
groups, wherein each Z is independently chosen from H, —OH, Cl, F, N₃, —NH₂, C₁₋₈ alkyl, C₂₋₈ alkenyl, C₂₋₈ alkynyl, C₆₋₁₄ aryl, —OC₁₋₈ alkyl, —OC₂₋₈ alkenyl, —OC₂₋₈ alkynyl, and —OC₆₋₁₄ aryl groups, wherein v is chosen from integers ranging from 0 to 3.

8. The method according to claim 2, wherein at least one R² is H.

9. The method according to claim 2, wherein at least one R² is F.

10. The method according to claim 2, wherein at least one R² is Cl.

11. The method according to claim 2, wherein at least one R² is chosen from —OY¹.

12. The method according to claim 2, wherein at least one R² is chosen from —OC(═O)Y¹.

13. The method according to claim 2, wherein at least one R² is chosen from —NY¹Y².

14. The method according to claim 5, wherein at least one R² is chosen from —NH(C═O)Y¹.

15. The method according to claim 2, wherein at least one R² is chosen from —NH(C═O)NY¹Y².

16. The method according to claim 13, wherein at least one of Y¹ and Y² is H.

17. The method according to claim 13, wherein each of Y¹ and Y² is H.

18. The method according to claim 14, wherein Y¹ is methyl.

19. The method according to claim 14, wherein Y¹ is phenyl.

20. The method according to claim 2 wherein at least one R² is chosen from

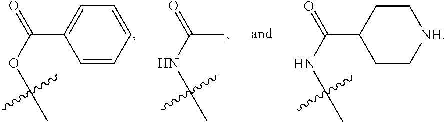

21. The method according to claim 18, wherein at least one R³ is chosen from

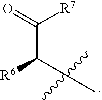

22. The method according to claim 2, wherein at least one R³ is chosen from

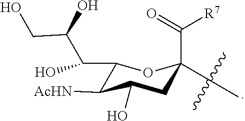

23. The method according to claim 21, wherein at least one R⁶ is chosen from

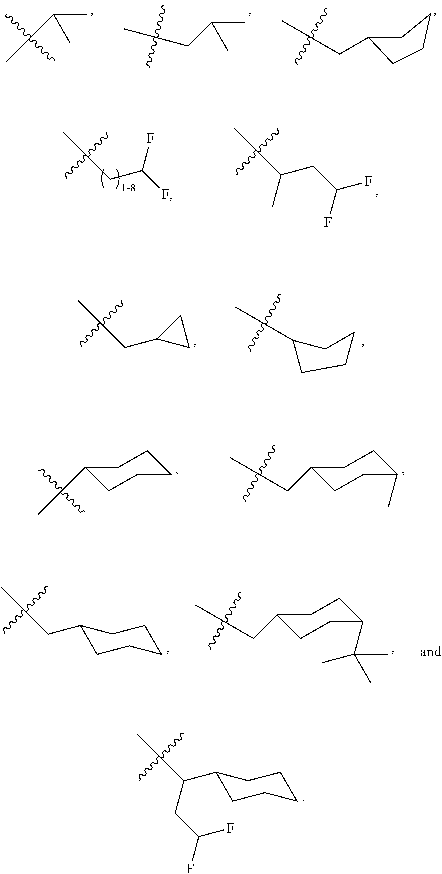

24. The method according to claim 23, wherein at least one R⁶ is chosen from

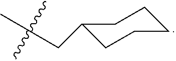 [SMILES: *CC1CCCCC1]

25. The method according to claim 23, wherein at least one R⁶ is chosen from

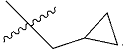 [SMILES: *CC1CC1]

26. The method according to claim 24, wherein at least one R⁷ is —OH.

27. The method according to claim 24, wherein at least one R⁷ is chosen from

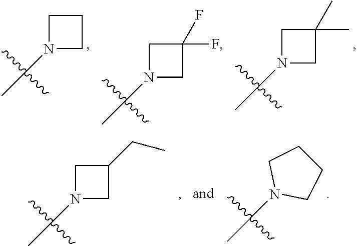

28. The method according to claim 27, wherein at least one R⁷ is chosen from

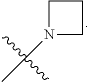 [SMILES: *N1CCC1]

29. The method according to claim 2, wherein at least one R⁴ is CH₃.

30. The method according to claim 2, wherein at least one R⁴ is chosen from CH₂F, CHF₂, and CF₃.

31. The method according to claim 2, wherein at least one R⁴ is CF₃.

32. The method according to claim 2, wherein the compound is chosen from compounds having the following Formula:

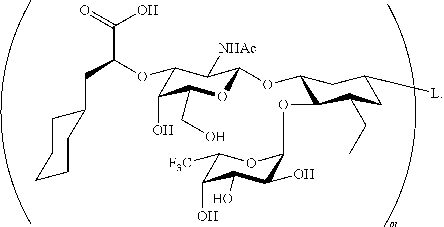

33. The method according to claim 2, wherein the compound is chosen from compounds having the following Formula:

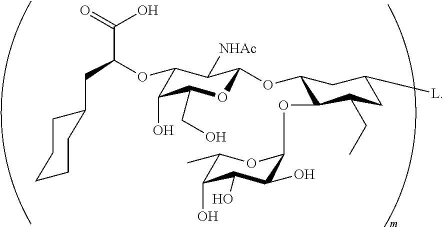

34. The method according to claim 2, wherein the compound is chosen from compounds having the following Formula:

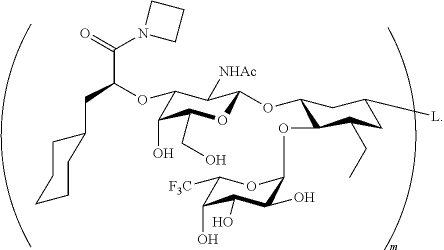

35. The method according to claim 2, wherein the compound is chosen from compounds having the following Formula:

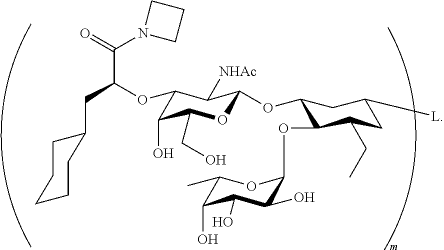

36. The method according to claim 35, wherein m is chosen from integers ranging from 2 to 8.

37. The method according to claim 36, wherein m is chosen from integers ranging from 2 to 4.

38. The method according to claim 36, wherein m is 4.

39. The method according to claim 36, wherein m is 3.

40. The method according to claim 36, wherein m is 2.

41. The method according to claim 36, wherein L is a dendrimer.

42. The method according to claim 40, wherein L is a chosen from 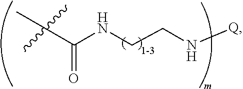
wherein Q is a chosen from 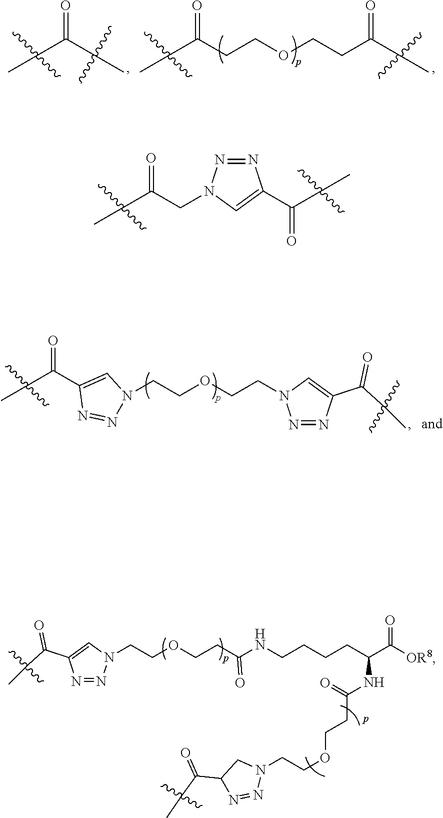
wherein R⁸ is chosen from H, C₁₋₈ alkyl, C₆₋₁₈ aryl, C₇₋₁₉ arylalkyl, and C₁₋₁₃ heteroaryl groups and each p, which may be identical or different, is independently chosen from integers ranging from 0 to 250.

43. The method according to claim 42, wherein R⁸ is H.

44. The method according to claim 42, wherein R⁸ is benzyl.

45. The method according to claim 42, wherein p is chosen from integers ranging from is 0 to 30.

46. The method according to claim 45, wherein p is 5.

47. The method according to claim 45, wherein p is 4.

48. The method according to claim 45, wherein p is 3.

49. The method according to claim 45, wherein p is 2.

50. The method according to claim 45, wherein p is 1.

51. The method according to claim 45, wherein p is 0.

52. (canceled)

53. (canceled)

54. The method according to claim 42, wherein L is chosen from:

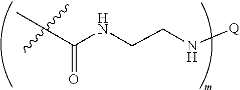

55. The method according to claim 42, wherein Q is:

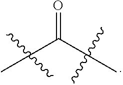 [SMILES: *C(*)=O]

56. The method according to claim 2, wherein the at least one compound is:

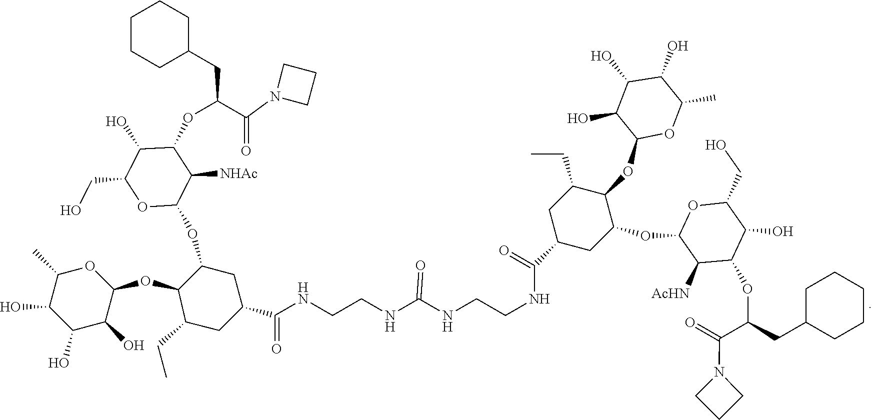 [SMILES: CC[C@H]1C[C@@H](C(=O)NCCNC(=O)NCCNC(=O)[C@@H]2C[C@H](CC)[C@@H](O[C@@H]3O[C@@H](C)[C@@H](O)[C@@H](O)[C@@H]3O)[C@H](O[C@@H]3O[C@H](CO)[C@H](O)[C@H](O[C@@H](CC4CCCCC4)C(=O)N4CCC4)[C@H]3NC(C)=O)C2)C[C@@H](O[C@@H]2O[C@H](CO)[C@H](O)[C@H](O[C@@H](CC3CCCCC3)C(=O)N3CCC3)[C@H]2NC(C)=O)[C@@H]1O[C@@H]1O[C@@H](C)[C@@H](O)[C@@H](O)[C@@H]1O]

57. The method according to any one of claims 2, 32-40, and 54-55, wherein the at least one compound is symmetrical.

58. The method according to any one of claims 2, 32-40, and 54-56, wherein at least one complication of sickle cell is vaso-occlusive crisis.
